SEQUENCE LISTING (62 sequences)

SEQ ID NO: 1 - protein, 340 aa, artificial
  E124 protein
MADSVKTFLQDLARGIKDSIWGICTISKLDARIQQKREEQRRRRASSVLAQRRAQSIERKQESEPRIVSRIFQCCAWNGGVFWFSLLLFYRVFIPVLQSVTARIIGDPSLHGDVWSWLEFFLTSIFSALWVLPLFVLSKVVNAIWFQDIADLAFEVSGRKPHPFPSVSKIIADMLFNLLLQALFLIQGMFVSLFPIHLVGQLVSLLHMSLLYSLYCFEYRWFNKGIEMHQRLSNIERNWPYYFGFGLPLAFLTAMQSSYIISGCLFSILFPLFIISANEAKTPGKAYLFQLRLFSLVVFLSNRLFHKTVYLQSALSSSTSAEKFPSPHPSPAKLKATAGH

SEQ ID NO: 2 - protein, 21 aa, artificial
  PRU P 3 allergen of PRUNUS PERSICA
ASSNGIRNVNNLARTPDRQAC

SEQ ID NO: 3 - protein, 138 aa, artificial
  tumor necrosis factor receptor (TNF)
VPAQWFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNWCA

SEQ ID NO: 4 - protein, 20 aa, artificial
  p38 gamma protein inhibitor
YGRKKRRQRRRARVPKETAL

SEQ ID NO: 5 - protein, 37 aa, artificial
  anti-cancer peptide-based therapeutic agent
RPMRLESFSACIWVKATDVLNKTILFSYGTKRNPYEI

SEQ ID NO: 6 - protein, 40 aa, artificial
  anti-cancer peptide-based therapeutic agent
GGGFDETLAFSGRLTGFNIWDSVLSNEEIRETGGAESCHI

SEQ ID NO: 7 - protein, 7 aa, artificial
  Tau protein
KHQPGGG

SEQ ID NO: 8 - protein, 7 aa, artificial
  Tau protein
KHVPGGG

SEQ ID NO: 9 - protein, 7 aa, artificial
  Tau protein
HHKPGGG

SEQ ID NO: 10 - protein, 7 aa, artificial
  Tau protein
THVPGGG

SEQ ID NO: 11 - protein, 34 aa, artificial
  SERCA calcium pump
MAEKESTSPHLMVPILLLVGWIVGCIIVIYIVFF

SEQ ID NO: 12 - protein, 35 aa, artificial
  SERCA calcium pump
MAEKAESTSPHLMVPILLLVGWIVGCIIVIYIVFF

SEQ ID NO: 13 - protein, 34 aa, artificial
  SERCA calcium pump
MAEKESTSPHLIVPILLLVGWIVGCIIVIYIVFF

SEQ ID NO: 14 - protein, 35 aa, artificial
  SERCA calcium pump
MAEKAESTSPHLIVPILLLVGWIVGCIIVIYIVFF

SEQ ID NO: 15 - protein, 9 aa, artificial
  excitatory neurotoxicity
YEKLLDTEI

SEQ ID NO: 16 - protein, 29 aa, artificial
  GLP1
HSQGTFTSDYSKYLDSRRAQDFVQWLMNT

SEQ ID NO: 17 - protein, 11 aa, artificial
  GLP1
EEPSSGAPPPS

SEQ ID NO: 18 - protein, 10 aa, artificial
  GLP1
EPSSGAPPPS

SEQ ID NO: 19 - protein, 6 aa, artificial
  GLP1
GAPPPS

SEQ ID NO: 20 - protein, 11 aa, artificial
  GLP1
GGPSSGAPPPS

SEQ ID NO: 21 - protein, 10 aa, artificial
  GLP1
GPSSGAPPPS

SEQ ID NO: 22 - protein, 9 aa, artificial
  GLP1
KRNKNPPPS

SEQ ID NO: 23 - protein, 8 aa, artificial
  GLP1
KRNKNPPS

SEQ ID NO: 24 - protein, 8 aa, artificial
  GLP1
KRNKPPIA

SEQ ID NO: 25 - protein, 8 aa, artificial
  GLP1
KRNKPPPA

SEQ ID NO: 26 - protein, 8 aa, artificial
  GLP1
KRNKPPPS

SEQ ID NO: 27 - protein, 9 aa, artificial
  GLP1
KSSGKPPPS

SEQ ID NO: 28 - protein, 9 aa, artificial
  GLP1
PESGAPPPS

SEQ ID NO: 29 - protein, 9 aa, artificial
  GLP1
PKSGAPPPS

SEQ ID NO: 30 - protein, 9 aa, artificial
  GLP1
PKSKAPPPS

SEQ ID NO: 31 - protein, 9 aa, artificial
  GLP1
PKSKAPPPS

SEQ ID NO: 32 - protein, 9 aa, artificial
  GLP1
PKSKEPPPS

SEQ ID NO: 33 - protein, 9 aa, artificial
  GLP1
PKSKEPPPS

SEQ ID NO: 34 - protein, 9 aa, artificial
  GLP1
PKSKQPPPS

SEQ ID NO: 35 - protein, 9 aa, artificial
  GLP1
PKSKSPPPS

SEQ ID NO: 36 - protein, 9 aa, artificial
  GLP1
PKSKSPPPS

SEQ ID NO: 37 - protein, 9 aa, artificial
  GLP1
PRNKNNPPS

SEQ ID NO: 38 - protein, 9 aa, artificial
  GLP1
PSKGAPPPS

SEQ ID NO: 39 - protein, 10 aa, artificial
  GLP1
PSSGAPPPSE

SEQ ID NO: 40 - protein, 9 aa, artificial
  GLP1
PSSGAPPPS

SEQ ID NO: 41 - protein, 9 aa, artificial
  GLP1
PSSGAPPPS

SEQ ID NO: 42 - protein, 10 aa, artificial
  GLP1
PSSGAPPPSS

SEQ ID NO: 43 - protein, 9 aa, artificial
  GLP1
PSSGEPPPS

SEQ ID NO: 44 - protein, 9 aa, artificial
  GLP1
PSSGKKPPS

SEQ ID NO: 45 - protein, 9 aa, artificial
  GLP1
PSSGKPPPS

SEQ ID NO: 46 - protein, 9 aa, artificial
  GLP1
PSSGKPPPS

SEQ ID NO: 47 - protein, 9 aa, artificial
  GLP1
PSSGSPPPS

SEQ ID NO: 48 - protein, 9 aa, artificial
  GLP1
PSSKAPPPS

SEQ ID NO: 49 - protein, 9 aa, artificial
  GLP1
PSSKEPPPS

SEQ ID NO: 50 - protein, 10 aa, artificial
  GLP1
PSSKGAPPPS

SEQ ID NO: 51 - protein, 9 aa, artificial
  GLP1
PSSKQPPPS

SEQ ID NO: 52 - protein, 9 aa, artificial
  GLP1
PSSKSPPPS

SEQ ID NO: 53 - protein, 7 aa, artificial
  GLP1
SGAPPPS

SEQ ID NO: 54 - protein, 8 aa, artificial
  GLP1
SSGAPPPS

SEQ ID NO: 55 - protein, 25 aa, artificial
  TP4
FIHHIIGGLFSAGKAIHRLIRRRRR

SEQ ID NO: 56 - protein, 20 aa, artificial
  BB barrier agent
LRVRLASHLRKLRKRLLRDA

SEQ ID NO: 57 - protein, 19 aa, artificial
  Telomerase inhibitor
RRRGGDASRSLPLPKRPRR

SEQ ID NO: 58 - protein, 19 aa, artificial
  Telomerase inhibitor
RRRGGEASRSLPLPKRPRR

SEQ ID NO: 59 - protein, 37 aa, artificial
  interferon
SSGSSSSSSSKAPPPSLPSPSRLPGPSDTPILPQNGS

SEQ ID NO: 60 - protein, 8 aa, artificial
  integrin binding
VGDLTYLK

SEQ ID NO: 61 - protein, 9 aa, artificial
  integrin binding
VGDLTYLKK

SEQ ID NO: 62 - protein, 25 aa, artificial
  Ziconotide
CKGKGAKCSRLMYDCCTGSCRSGKC

(19) United States
(12) Patent Application Publication
(10) Pub. No.: US 2022/0105190 A1
(43) Pub. Date: Apr. 7, 2022

(54) METHODS OF USING GLYCOPOLYSIALYLATED THERAPEUTIC PROTEINS

(71) Applicant: XENETIC BIOSCIENCES, INC., Lexington, MA (US)

(72) Inventor: Curtis LOCKSHIN, Lexington, MA (US)

(73) Assignee: Lipoxen Technologies Limited, Lexington, MA (US)

(21) Appl. No.: 17/428,574

(22) PCT Filed: Feb. 4, 2020
(86) PCT No.: PCT/US2020/016500

§ 371 Date: Aug. 4, 2021

Related U.S. Application Data
(60) Provisional application No. 62/801,013, filed on Feb. 4, 2019.

(51) Int. Cl. A61K 47/61; A61K 45/06; A61K 38/48; A61P 7/02

(52) U.S. Cl. CPC A61K 47/61; A61P 7/02; A61K 38/4833; A61K 45/06

(57) ABSTRACT

Novel proteins and compounds conjugated with polysialic acid (PSA) are provided herein. Also provided are methods of using these compounds and methods of treatment of various diseases and disorders. The novel compounds provided herein have improved pharmacodynamic and/or pharmacokinetic properties, improved effectiveness, and other desirable properties.

### RELATED APPLICATIONS

This application claims priority and is entitled to the filing date of U.S. provisional patent application Ser. No. 62/801,013 filed on Feb. 4, 2019. The contents of the aforementioned application are incorporated herein by reference.

### FIELD OF THE INVENTION

The present invention relates to proteins and other biological compounds conjugated with water soluble polymers, in particular novel protein (e.g. glycoprotein) conjugates obtained upon conjugation to water soluble polymers such as PSA. The invention also relates to methods of use and treatment using novel conjugated proteins and compounds and formulations described herein.

### BACKGROUND OF THE INVENTION

Conjugation of polypeptide drugs such as by PEGylation or polysialylation protects them from degradation in the blood circulation and thus improves their pharmacodynamic and pharmacokinetic profiles (Harris and Chess, Nat Rev Drug Discov. 2003;2:214-21; S. Jain, D. Hreczuk-Hirst, P. Laing and G. Gregoriadis, Drug Delivery Systems and Sciences, 4 (No 1): 3-9, 2004.). Sialic acids (also called N-acetyl neuraminic acids) and polysialic acids are found widely distributed in animal tissues and to a lesser extent in other species ranging from plants and fungi to yeasts and bacteria, mostly in glycoproteins and gangliosides. The abbreviation “PSA” used herein refers to the term “polysialic acid”. Similarly, the term “mPSA” used herein refers to the term “modified polysialic acid”.

PSAs consist of polymers (generally homopolymers) of N-acetylneuraminic acid. The secondary amino group normally bears an acetyl group, but it may instead bear a glycolyl group. Possible substituents on the hydroxyl groups include acetyl, lactyl, ethyl, sulfate, and phosphate groups.

PSAs and mPSAs generally comprise linear polymers consisting essentially of N-acetylneuraminic acid moieties linked by 2,8- or 2,9-glycosidic linkages or combinations of these (e.g. alternating 2,8- and 2,9-linkages). In particularly preferred PSAs and mPSAs, the glycosidic linkages are cc-2,8. Such PSAs and mPSAs are conveniently derived from colominic acids, and are referred to herein as “CAs” and “mCAs”. Typical PSAs and mPSAs comprise at least 2, preferably at least 5, more preferably at least 10 and most preferably at least 20 N-acetylneuraminic acid moieties. Thus, they may comprise from 5 to 500 N-acetylneuraminic acid moieties, preferably from 10 to 300 N-acetylneuraminic acid moieties. PSAs and CAs can be polymers comprising different sugar moieties. They can be copolymers. PSAs and CAs preferably are essentially free of sugar moieties other than N-acetylneuraminic acid. PSAs and CAs preferably comprise at least 90%, more preferably at least 95% and most preferably at least 98% N-acetylneuraminic acid moieties.

Where PSAs and CAs comprise moieties other than N-acetylneuraminic acid (as, for example in mPSAs and mCAs) these are preferably located at one or both of the ends of the polymer chain. Such “other” moieties may, for example, be moieties derived from terminal N-acetylneuraminic acid moieties by oxidation or reduction. For example, a mPSAs and/or mCAs can be made in which the non-reducing terminal N-acetylneuraminic acid unit is converted to an aldehyde group by reaction with sodium periodate (see WO-A-0187922). Alternatively, for example, mPSAs and mCAs ca be utilized in which the reducing terminal N-acetylneuraminic acid unit is subjected to reduction to reductively open the ring at the reducing terminal N-acetylneuraminic acid unit to form a vicinal diol group which is oxidixed to convert the vicinal diol group to an aldehyde group (see WO 2005/016974).

The preparation of conjugates by forming a covalent linkage between the water soluble polymer and a protein (e.g. a therapeutic protein) can be carried out by a variety of chemical methods. One approach for coupling PSA to proteins is the conjugation of the polymers via the carbohydrate moieties of the protein. Vicinal hydroxyl (OH) groups of carbohydrates in proteins can be easily oxidized with sodium periodate (NaIO₄) to form active aldehyde groups (Rothfus and Smith, J Biol Chem 1963; 238:1402-10; van Lenten and Ashwell, J Biol Chem 1971;246:1889-94). Subsequently the polymer can be coupled to the aldehyde groups of the carbohydrate by use of reagents containing, for example, an active hydrazide group (Wilchek M and Bayer E A, Methods Enzymol 1987;138:429-42). A more recent technology is the use of reagents containing aminooxy groups which react with aldehydes to form oxime linkages (WO 96/40662, WO2008/025856).

Notwithstanding the methods available of conjugating water soluble polymers to therapeutic proteins, there remains a need to develop materials and methods for conjugating water soluble polymers to a wide variety of carbohydrate-containing proteins and compounds in order to improve the compound's pharmacodynamic and/or pharmacokinetic properties. Further, there remains an enormous unmet need to develop and test a broad range of drugs conjugated with a PSA or mPSA to determine whether they have beneficial properties. In this application, we address this deficiency by providing and testing numerous particular compounds conjugated with a PSA or mPSA.

### SUMMARY OF THE INVENTION

The present invention provides compounds (e.g. proteins) conjugated with water soluble polymers, and in particular novel compounds conjugated with a PSA or mPSA and methods of use of such compounds.

The novel compounds provided herein preferably have improved pharmacodynamic and/or pharmacokinetic properties and other desirable properties. The compounds provided herein are typically proteins, and more typically glycoproteins other than a blood coagulation proteins.

A preferred non-limiting water-soluble polymer used is PSA or mPSA, which are referred to herein simply as PSA in reference to the embodiments herein. The water-soluble polymer used can also be, but is not limited to, polyethylene glycol (PEG), branched PEG, PEG derivative, CA, mCA, hydroxyethyl cellulose (HEC), dextrin, polyoxazoline, carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG) polyoxazoline, poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(l-hydroxymethylethylene hydroxymethylformal) (PHF), 2-methacryloyloxy-2′-ethyltrimethylammoniumphosphate (MPC).

In further embodiments of the invention illustrated in examples below, the water soluble polymer is polysialic acid (PSA) or a modified PSA (mPSA). The PSA or mPSA may have a molecular weight range of 350 Da to 120,000 Da, 500 Da to 100,000 Da,1000 Da to 80,000 Da, 1500 Da to 60,000 Da, 2,000 Da to 45,000 Da or 3,000 Da to 35,000 Da. The PSA or mPSA may be colominic acid or modified colominic acid. The PSA or mPSA is typically comprised of about 2-500 or 10-300 sialic acid units, however other numbers of sialic acid units are envisioned. Also described herein are materials and methods for conjugating a water soluble polymer to a wide variety of carbohydrate-containing compounds in order to improve the compound's pharmacodynamic and/or pharmacokinetic properties. The compounds described herein can be prepared by various methods described herein. A non-limiting example comprises contacting the oxidized carbohydrate moiety with a water soluble polymer under conditions that allow conjugation, wherein said water soluble polymer contains an aminooxy group and an oxime linkage is formed between the oxidized carbohydrate moiety and the aminooxy group on the water soluble polymer, or wherein said water soluble polymer contains a hydrazide group and a hydrazone linkage is formed between the oxidized carbohydrate moiety and the hydrazide group on the water soluble polymer. The water soluble polymer may also be oxidized to form an aldehyde group on a terminal sialic acid unit of the water soluble polymer, followed by reacting the oxidized water soluble polymer with an aminooxy linker. Thus in certain embodiments, the water soluble polymer is prepared by reacting an activated aminooxy linker with oxidized water soluble polymer where the linker is a homobifunctional or heterobifunctional linker. The homobifunctional linker can have the general formula NH₂[OCH₂CH₂]ₙONH₂, wherein n=1-50, preferably 1-11, more preferably 1-6. Other suitable linkers are further described in WO2011/012850 by Jain et al., entitled “Glycopolysialylation of non-blood coagulation proteins’ incorporated by reference herein.

Another non-limiting example of methods used to produce certain novel compounds conjugated to water soluble polymers provided herein comprise oxidizing the water soluble polymer to form an aldehyde group on a terminal unit of the water soluble polymer, e.g. a terminal sialic acid unit of the PSA or mPSA, and reacting the oxidized water soluble polymer with an aminooxy linker. Certain novel compounds may be formed where a hydrazide group is formed on a water soluble polymer (e.g. PSA or mPSA) by reacting the oxidized water soluble polymer with a hydrazide linker. The hydrazide linker can suitably be adipic acid dihydrazide or hydrazine. Certain novel compounds may be formed by reducing an oxime or hydrazone linkage in a conjugated compound (e.g. protein). Other novel compounds described herein are prepared by linkages and using linkers that are described in detail in U.S. Ser. Nos. 10/276,552, 10/568,111, 11/660,128, 11/816,823, 12/375,012, 12/843,284, U.S. Pat. Nos. 6,166,687, 8,217,154, and 9,795,683, all incorporated by reference in their entirety.

### DETAILED DESCRIPTION OF THE INVENTION

As used herein “biologically active derivative” or “biologically active variant” includes any derivative or variant of a molecule having substantially the same functional and/or biological properties of said molecule, such as binding properties, and/or the same structural basis, such as a peptidic backbone or a basic polymeric unit.

The term “gene expression disorder” refers to numerous genetic disorders, caused by the mutation of a single gene are known and candidates for RNA therapeutic approaches. Disorders caused by single-gene mutations, like cystic fibrosis, hemophilia and many others, can be dominant or recessive with respect to the likelihood that a certain trait will appear in the offspring. While a dominant allele manifests a phenotype in individuals who have only one copy of the allele, for a recessive allele the individual must have two copies, one from each parent to become manifest. In contrast, polygenic disorders are caused by two or more genes and the manifestation of the respective disease is often fluent and associated to environmental factors. Examples for polygenic disorders are hypertension, elevated cholesterol level, cancer, neurodegenerative disorders, mental illness and others. Also, in these cases therapeutic RNA representing one or more of these genes may be beneficial to those patients. Furthermore, a genetic disorder must not have been passed down from the parents' genes, but can also be caused by new mutations. Also, in these cases therapeutic RNA representing the correct gene sequence may be beneficial to the patients. Other gene expression disorder include hypertension, elevated cholesterol level, cancer, neurodegenerative disorders, mental illness, cystic fibrosis, hemophilia (or other blood clotting disease), europsychiatric disorders, such as schizophrenia, bipolar disorder, major depression, Parkinson's disease, Alzheimer's disease and autism spectrum disorders, Albinism, Angelman syndrome, Ankylosing spondylitis, Apert syndrome, Charcot-Marie-Tooth disease, Congenital adrenal hyperplasia, Cystic fibrosis, Down syndrome, Achondroplasia, Alpha-1 Antitrypsin Deficiency, Antiphospholipid Syndrome, Attention Deficit Hyperactivity Disorder, Autism, Autosomal Dominant Polycystic Kidney Disease, Charcot-Marie-Tooth Disease, Cri du Chat Syndrome, Crohn's Disease, Cystic Fibrosis, Dercum Disease, Duane Syndrome, Duchenne Muscular Dystrophy, Factor V Leiden Thrombophilia, Familial Hypercholesterolemia, Familial Mediterranean Fever, Fragile X Syndrome, Gaucher Disease, Hemochromatosis, Holoprosencephaly, Huntington's Disease, Inborn Errors of Metabolism, Klinefelter Syndrome, Marfan Syndrome, Methylmalonic Acidemia, Myotonic Dystrophy, Neurofibromatosis, Noonan Syndrome, Osteogenesis Imperfecta, Parkinson's Disease, Phenylketonuria, Poland Anomaly, Porphyria, Progeria, Retinitis Pigmentosa, Severe Combined Immunodeficiency, Sickle Cell Disease, Spinal Muscular Atrophy, Tay-Sachs Disease, Thalassemia, Trimethylaminuria, Turner Syndrome, Velocardiofacial Syndrome and Wilson Disease. An online catalog with presently 22,993 entries of Human Genes and Genetic Disorders together with their respective genes and a description of their phenotypes are available at the ONIM (Online Mendelian Inheritance in Man) webpage (http://omim.org); sequences of each are available from the Uniprot database (http://www.uniprot.org).

Embodiments include methods of treating a gene expression disorder. The method can include a step of administering an effective amount of a PSA-nucleic acid conjugate to a patient. The PSA-conjugate can include PSA covalently linked to an RNA oligonucleotide selected from double stranded RNA, single-stranded RNA or short interfering RNA (siRNA). Alternatively, the PSA-conjugate can comprise PSA covalently linked to an RNA oligonucleotide via cleavable linker moiety. In other embodiments, the PSA molecule is conjugated, optionally via a linker, to at least one RNA molecule at the RNA 3′ terminal base. Alternatively, the PSA-conjugate can comprise PSA covalently linked to an RNA oligonucleotide wherein RNA oligonucleotide comprises coding region coding for a polypeptide or its complementary sequence wherein polypeptide is VEGF, Apolipoprotein B, Exon 51 of dystrophin, SMN2, Transthyretin, CEP290c.2991+1655A>G Mutation, KRAS, Complement 5 (C5) protein, EphA2, CTGF, TRPV1, LDHA, TGF-β1, Cox-2, KRAS G12D, P53, Caspase-2, Antithrombin, FANCA, Coagulation Factor VIII, Coagulation Factor IX, ANK1, PIG-A, UROD, Adenosine deaminase, JAK3, RAG1/2, Artemis, IL7R-α, IL-2Rγ, T-cell surface glycoprotein CD3 delta chain, CD3.epsilon, CDKN2, NF1, NF2, LIM kinase, elastin, ALDP, CFTR, hepcidin, ABCA3, surfactant protein B, ADAMTS13, alpha.1-antitrypsin or GAA.

The term “ailment” or “disease” refers to a particular abnormal condition that negatively affects the structure or function of part or all of a person that is not due to any immediate external injury. Diseases are often known to be medical conditions that are associated with specific symptoms and signs. A disease may be caused by external factors such as pathogens or by internal dysfunctions. For example, internal dysfunctions of the immune system can produce a variety of different diseases, including various forms of immunodeficiency, hypersensitivity, allergies and autoimmune disorders.

An “analog,” “variant” or “derivative” is a compound substantially similar in structure and having the same biological activity, albeit in certain instances to a differing degree, to a naturally-occurring molecule. For example, a polypeptide variant refers to a polypeptide sharing substantially similar structure and having the same biological activity as a reference polypeptide. Variants or analogs differ in the composition of their amino acid sequences compared to the naturally-occurring polypeptide from which the analog is derived, based on one or more mutations involving (i) deletion of one or more amino acid residues at one or more termini of the polypeptide and/or one or more internal regions of the naturally-occurring polypeptide sequence (e.g., fragments), (ii) insertion or addition of one or more amino acids at one or more termini (typically an “addition” or “fusion”) of the polypeptide and/or one or more internal regions (typically an “insertion”) of the naturally-occurring polypeptide sequence or (iii) substitution of one or more amino acids for other amino acids in the naturally-occurring polypeptide sequence. By way of example, a “derivative” refers to a polypeptide sharing the same or substantially similar structure as a reference polypeptide that has been modified, e.g., chemically.

Variant or analog polypeptides include insertion variants, wherein one or more amino acid residues are added to a protein amino acid sequence of the invention. Insertions may be located at either or both termini of the protein, and/or may be positioned within internal regions of the protein amino acid sequence. Insertion variants, with additional residues at either or both termini, include for example, fusion proteins and proteins including amino acid tags or other amino acid labels. In one aspect, the protein molecule optionally contains an N-terminal Met, especially when the molecule is expressed recombinantly in a bacterial cell such as E. coli.

In deletion variants, one or more amino acid residues in a protein or polypeptide as described herein are removed. Deletions can be affected at one or both termini of the protein or polypeptide, and/or with removal of one or more residues within the protein amino acid sequence. Deletion variants, therefore, include fragments of a protein or polypeptide sequence.

In substitution variants, one or more amino acid residues of a protein or polypeptide are removed and replaced with alternative residues. In one aspect, the substitutions are conservative in nature and conservative substitutions of this type are well known in the art. Alternatively, the invention embraces substitutions that are also non-conservative. Exemplary conservative substitutions are described in Lehninger, [Biochemistry, 2nd Edition; Worth Publishers, Inc., New York (1975), pp.71-77], incorporated by reference herein.

The pharmacological and immunological properties of carbohydrate-containing compounds, such as glycoproteins other than blood coagulations proteins can be improved by chemical modification and conjugation with water soluble polymer, in particular PEG or PSA or mPSA. The properties of the resulting conjugates generally strongly depend on the structure and the size of the polymer. Thus, polymers with a defined and narrow size distribution are usually preferred. PSA and mPSA, used in specific examples, can be purified in such a manner that results in a final PSA preparation with a narrow size distribution.

### Glycoproteins and Other Protein Targets

As described herein, glycoproteins other than blood coagulation proteins including, but not limited to cytokines such as interleukins, alpha-, beta-, and gamma-interferons, colony stimulating factors including granulocyte colony stimulating factors, fibroblast growth factors, platelet derived growth factors, phospholipase-activating protein (PUP), insulin, plant proteins such as lectins and ricins, tumor necrosis factors and related alleles, soluble forms of tumor necrosis factor receptors, interleukin receptors and soluble forms of interleukin receptors, growth factors, tissue growth factors, transforming growth factors such as TGFαs or TGFβs and epidermal growth factors, hormones, somatomedins, pigmentary hormones, hypothalamic releasing factors, antidiuretic hormones, prolactin, chorionic gonadotropin, follicle-stimulating hormone, oxyntomodulin and oxyntomodulin-like peptides, thyroid-stimulating hormone, tissue plasminogen activator, and immunoglobulins such as IgG, IgE, IgM, IgA, and IgD, monoclonal antibodies, erythropoietin (EPO), blood factors other than blood coagulation proteins, galactosidases, α-galactosidases, β-galactosidases, DNAses, fetuin, fragments thereof, and any fusion proteins comprising any of the above mentioned proteins or fragments thereof together with therapeutic glycoproteins in general are contemplated by the invention.

In numerous particular embodiments provided herein, proteins, polypeptides and peptides which are involved in a wide variety of regulatory and signal transduction pathways are conjugated to make protein-PSA conjugates. In certain embodiments, the proteins, peptides, and other compounds used to make novel protein-PSA conjugates are selected from the following: Factor IX (FIX), Factor VIII (FVIII), Factor VIIa (FVIIa), Von Willebrand Factor (VWF), Factor FV (FV), Factor X (FX), Factor XI (FXI), Factor XII (FXII), thrombin (FII), thrombomodulin (and analogs, e.g. Solulin), protein C, protein S, tPA, PAI-1, tissue factor (TF), ADAMTS 13 protease, IL-1 alpha, IL-1 β, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-β, IFN-gamma, IFN-omega, IL-7, IL-8, IL-9, IL-10, IL-12, IL-13, IL-14, IL-15, IL-16, IL-17, IL-18, IL-19, IL-20, IL-21, IL-22, IL-23, IL-24, IL-31, IL-32 alpha, IL-33, thrombopoietin (TPO), Ang-1, Ang-2, Ang-4, Ang-Y, angiopoietin-like polypeptide 1 (ANGPTL1), angiopoietin-like polypeptide 2 (ANGPTL2), angiopoietin-like polypeptide 3 (ANGPTL3), angiopoietin-like polypeptide 4 (ANGPTL4), angiopoietin-like polypeptide 5 (ANGPTL5), angiopoietin-like polypeptide 6 (ANGPTL6), angiopoietin-like polypeptide 7 (ANGPTL7), vitronectin, vascular endothelial growth factor (VEGF), angiogenin, activin A, activin B, activin C, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, bone morphogenic protein receptor II, brain derived neurotrophic factor, cardiotrophin-1, ciliary neutrophic factor, ciliary neutrophic factor receptor, cripto, cryptic, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epidermal growth factor, epigen, epiregulin, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor 11, fibroblast growth factor 12, fibroblast growth factor 13, fibroblast growth factor 16, fibroblast growth factor 17, fibroblast growth factor 19, fibroblast growth factor 20, fibroblast growth factor 21, fibroblast growth factor acidic, fibroblast growth factor basic, glial cell line-derived neutrophic factor receptor α1, glial cell line-derived neutrophic factor receptor α 2, growth related protein, growth related protein α, growth related protein β, growth related protein .gamma., heparin binding epidermal growth factor, hepatocyte growth factor, hepatocyte growth factor receptor, hepatoma-derived growth factor, insulin-like growth factor I, insulin-like growth factor receptor, insulin-like growth factor II, insulin-like growth factor binding protein, keratinocyte growth factor, leukemia inhibitory factor, leukemia inhibitory factor receptor a, endomorphins (1 & 2), neuropeptides, nerve growth factor nerve growth factor receptor, neuropoietin, neurotrophin-3, neurotrophin-4, oncostatin M (OSM), placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor a, platelet derived growth factor receptor β, pre-B cell growth stimulating factor, stem cell factor (SCF), stem cell factor receptor, TNF, TNF0, TNF1, TNF2, transforming growth factor a, transforming growth factor β, transforming growth factor β1, transforming growth factor β 1.2, transforming growth factor β 2, transforming growth factor β3, transforming growth factor β5, latent transforming growth factor β 1, transforming growth factor β binding protein I, transforming growth factor β binding protein II, transforming growth factor β binding protein III, thymic stromal lymphopoietin (TSLP), tumor necrosis factor receptor type I, tumor necrosis factor receptor type II, urokinase-type plasminogen activator receptor, phospholipase-activating protein (PUP), insulin, lectin ricin, prolactin, chorionic gonadotropin, follicle-stimulating hormone, thyroid-stimulating hormone, tissue plasminogen activator, IgG, IgE, IgM, IgA, and IgD, a-galactosidase, β-galactosidase, DNAse, fetuin, leutinizing hormone, estrogen, insulin, albumin, lipoproteins, fetoprotein, transferrin, thrombopoietin, urokinase, integrin, thrombin, thrombomodulin, leptin, adalimumab, denosumab, or etanercept. These proteins and particular methods of making these compounds are described in further detail in U.S. Pat. No. 9,731,024 entitled ‘Nucleophilic catalyst for oxime linkage’, is incorporated by reference herein.

In certain embodiments, the proteins, peptides, and other compounds used to make novel protein-PSA conjugates are selected from the following: factor IX (FIX), Factor VIII (FVIII), Factor VIIa (FVIIa), von Willebrand Factor (VWF), Factor FV (FV), Factor X (FX), Factor XI (FXI), Factor XII (FXII), thrombin (FII), protein C, protein S, tPA, PAI-1, tissue factor (TF), ADAMTS 13 protease, IL-1 alpha, IL-1 β, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-β, IFN-gamma, IFN-omega, IL-7, IL-8, IL-9, IL-10, IL-12, IL-13, IL-14, IL-15, IL-16, IL-17, IL-18, IL-19, IL-20, IL-21, IL-22, IL-23, IL-24, IL-31, IL-32 alpha, IL-33, thrombopoietin (TPO), Ang-1, Ang-2, Ang-4, Ang-Y, angiopoietin-like polypeptide 1 (ANGPTL1), angiopoietin-like polypeptide 2 (ANGPTL2), angiopoietin-like polypeptide 3 (ANGPTL3), angiopoietin-like polypeptide 4 (ANGPTL4), angiopoietin-like polypeptide 5 (ANGPTL5), angiopoietin-like polypeptide 6 (ANGPTL6), angiopoietin-like polypeptide 7 (ANGPTL7), vitronectin, vascular endothelial growth factor (VEGF), angiogenin, activin A, activin B, activin C, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, bone morphogenic protein receptor II, brain derived neurotrophic factor, cardiotrophin-1, ciliary neutrophic factor, ciliary neutrophic factor receptor, cripto, cryptic, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epidermal growth factor, epigen, epiregulin, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor 11, fibroblast growth factor 12, fibroblast growth factor 13, fibroblast growth factor 16, fibroblast growth factor 17, fibroblast growth factor 19, fibroblast growth factor 20, fibroblast growth factor 21, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein .gamma., heparin binding epidermal growth factor, hepatocyte growth factor, hepatocyte growth factor receptor, hepatoma-derived growth factor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neuropoietin, neurotrophin-3, neurotrophin-4, oncostatin M (OSM), placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor (SCF), stem cell factor receptor, TNF, TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, thymic stromal lymphopoietin (TSLP), tumor necrosis factor receptor type I, tumor necrosis factor receptor type II, urokinase-type plasminogen activator receptor, phospholipase-activating protein (PUP), insulin, lectin ricin, prolactin, chorionic gonadotropin, follicle-stimulating hormone, thyroid-stimulating hormone, tissue plasminogen activator, IgG, IgE, IgM, IgA, and IgD, α-galactosidase, β-galactosidase, DNAse (e.g. DNAse I), fetuin, leutinizing hormone, estrogen, insulin, albumin, lipoproteins, fetoprotein, transferrin, thrombopoietin, urokinase, integrin, thrombin, thrombomodulin, leptin, Humira (adalimumab), Prolia (denosumab), or a biologically active fragment, derivative or variant thereof. These proteins and particular methods of making these compounds are described in further detail in US Patent Application entitled ‘Materials and methods for conjugating a water soluble fatty acid derivative to a protein’ published as 20120190096, which is incorporated by reference herein.

In certain embodiments, the proteins, peptides, and other compounds used to make novel protein-PSA conjugates are selected from the following: aspariginase, amdoxovir (DAPD), antide, becaplermin, calcitonins, cyanovirin, denileukin diftitox, erythropoietin (EPO), EPO agonists (e.g., peptides from about 10-40 amino acids in length and comprising a particular core sequence as described in WO 96/40749), dornase alpha, erythropoiesis stimulating protein (NESP), coagulation factors such as Factor V, Factor VII, Factor Vila, Factor VIII, Factor IX, Factor X, Factor XII, Factor XIII, von Willebrand factor; ceredase, cerezyme, alpha-glucosidase, collagen, cyclosporin, alpha defensins, β defensins, exedin-4, granulocyte colony stimulating factor (GCSF), thrombopoietin (TPO), alpha-1 proteinase inhibitor, elcatonin, granulocyte macrophage colony stimulating factor (GMCSF), fibrinogen, filgrastim, growth hormones human growth hormone (hGH), growth hormone releasing hormone (GHRH), GRO-β, GRO-β antibody, bone morphogenic proteins such as bone morphogenic protein-2, bone morphogenic protein-6, OP-1, acidic fibroblast growth factor, basic fibroblast growth factor, CD-40 ligand, heparin, human serum albumin, low molecular weight heparin (LMWH), interferons such as interferon alpha, interferon β, interferon gamma, interferon omega, interferon tau, consensus interferon; interleukins and interleukin receptors such as interleukin-1 receptor, interleukin-2, interluekin-2 fusion proteins, interleukin-1 receptor antagonist, interleukin-3, interleukin-4, interleukin-4 receptor, interleukin-6, interleukin-8, interleukin-12, interleukin-13 receptor, interleukin-17 receptor; lactoferrin and lactoferrin fragments, luteinizing hormone releasing hormone (LHRH), insulin, pro-insulin, insulin analogues (e.g., mono-acylated insulin as described in U.S. Pat. No. 5,922,675), amylin, C-peptide, somatostatin, somatostatin analogs including octreotide, vasopressin, follicle stimulating hormone (FSH), influenza vaccine, insulin-like growth factor (IGF), insulintropin, macrophage colony stimulating factor (M-CSF), plasminogen activators such as alteplase, urokinase, reteplase, streptokinase, pamiteplase, lanoteplase, and teneteplase; nerve growth factor (NGF), osteoprotegerin, platelet-derived growth factor, tissue growth factors, transforming growth factor-1, vascular endothelial growth factor, leukemia inhibiting factor, keratinocyte growth factor (KGF), glial growth factor (GGF), T Cell receptors, CD molecules/antigens, tumor necrosis factor (TNF), monocyte chemoattractant protein-1, endothelial growth factors, parathyroid hormone (PTH), glucagon-like peptide, somatotropin, thymosin alpha 1, thymosin alpha 1 IIb/IIIa inhibitor, thymosin β 10, thymosin β 9, thymosin β 4, alpha-1 antitrypsin, phosphodiesterase (PDE) compounds, VLA-4 (very late antigen-4), VLA-4 inhibitors, bisphosponates, respiratory syncytial virus antibody, cystic fibrosis transmembrane regulator (CFTR) gene, deoxyreibonuclease (Dnase), bactericidal/permeability increasing protein (BPI), and anti-CMV antibody. Exemplary monoclonal antibodies include etanercept (a dimeric fusion protein consisting of the extracellular ligand-binding portion of the human 75 kD TNF receptor linked to the Fc portion of IgG1), abciximab, afeliomomab, basiliximab, daclizumab, infliximab, ibritumomab tiuexetan, mitumomab, muromonab-CD3, iodine 131 tositumomab conjugate, olizumab, rituximab, and trastuzumab (herceptin). These proteins and particular methods of making these compounds are described in further detail in US Patent Application entitled ‘Multi-arm polymer prodrugs’ published as US2018021444, which is incorporated by reference herein. Examples of used to make novel protein-PSA conjugates include, without limitation, erythropoietin (EPO) such as recombinant human EPO (rhEPO), colony-stimulating factors (CSF), such as G-CSF like recombinant human G-CSF (rhG-CSF), alpha-Interferon (IFN alpha), β-Interferon (IFN β) or gamma-Interferon (IFN gamma), such as IFN alpha and IFN β like recombinant human IFN alpha or IFN β (rhIFN alpha or rhIFN β), interleukines, e. g. IL-1 to IL-18 such as IL-2 or IL-3 like recombinant human IL-2 or IL-3 (rhIL-2 or rhIL-3), serum proteins such as coagulation factors II-XIII like factors VII, VIII, IX, alphal-antitrypsin (A1AT), activated protein C (APC), plasminogen activators such as tissue-type plasminogen activator (tPA), such as human tissue plasminogen activator (hTPA), AT III such as recombinant human AT III (rhAT III), myoglobin, albumin such as bovine serum albumin (BSA), growth factors, such as epidermal growth factor (EGF), thrombocyte growth factor (PDGF), fibroblast growth factor (FGF), brain-derived growth factor (BDGF), nerve growth factor (NGF), B-cell growth factor (BCGF), brain-derived neurotrophic growth factor (BDNF), ciliary neurotrophic factor (CNTF), transforming growth factors such as TGF alpha or TGF β, BMP (bone morphogenic proteins), growth hormones such as human growth hormone, tumor necrosis factors such as TNF alpha or TNF β, somatostatine, somatotropine, somatomedines, hemoglobin, hormones or prohormones such as insulin, gonadotropin, melanocyte-stimulating hormone (alpha-MSH), triptorelin, hypthalamic hormones such as antidiuretic hormones (ADH and oxytocin as well as releasing hormones and release-inhibiting hormones, parathyroid hormone, thyroid hormones such as thyroxine, thyrotropin, thyroliberin, prolactin, calcitonin, glucagon, glucagon-like peptides (GLP-1, GLP-2 etc.), exendines such as exendin-4 and Extendatide, leptin, vasopressin, gastrin, secretin, integrins, glycoprotein hormones (e. g. LH, FSH etc.), melanoside-stimulating hormones, lipoproteins and apo-lipoproteins such as apo-B, apo-E, apo-La, immunoglobulins such as IgG, IgE, IgM, IgA, IgD and fragments thereof, hirudin, tissue-pathway inhibitor, plant proteins such as lectin or ricin, bee-venom, snalce-venom, immunotoxins, antigen E, alpha-proteinase inhibitor, ragweed allergen, melanin, oligolysine proteins, RGD proteins or optionally corresponding receptors for one of these proteins; or a functional derivative or fragment of any of these proteins or receptors. Preferred enzymes are, e.g., carbohydrate-specific enzymes, proteolytic enzymes, oxidases, oxidoreductases, transferases, hydrolases, lyases, isomerases, kinases and ligases. Specific non-limiting examples are asparaginase, arginase, arginin deaminase, adenosis deaminase, glutaminase, glutaminase-asparaginase, phenylalanin, tryptophanase, tyrosinase, superoxide dismutase (SOD), endotoxinase, catalase, peroxidase, kallikrein, trypsin, chymotrypsin, elastase, thermolysis, lipase, uricase, adenosine diphosphatase, purine nucleoside phosphorylase, bilirubin oxidase, glucose oxidase, glucose, gluconate oxidase, galactosidase, glucocerebrosidase, glucuronidase, hyaluronidase, tissue factor, streptokinase, urokinase, MAP-kinases, DNAses (e.g. DNAse I), RNAses, lactoferrin and functional derivatives or fragments thereof. These proteins and particular methods of making these compounds are described in further detail in US Patent Application entitled ‘Conjugates of hydroxyalkyl starch and a protein, prepared by reductive amination’ published as U.S./Publication No. 2007134197, which is incorporated by reference herein.

In another aspect, antibody-drug conjugates are conjugated to make antibody-drug-PSA conjugates. These antibodies and particular methods of making these compounds are described in detail in PCT entitled ‘Antibody-drug conjugates and therapeutic methods using the same’ published as WO2018002902, which is incorporated by reference herein. In these embodiments, one suitable antibody-drug conjugate comprises an antibody covalently bound to a linker molecule which is in turn covalently bound to one or more drugs capable of binding to an HIV envelope glycoprotein. Another suitable antibody to make a conjugate is one that binds to the HIV envelope glycoprotein at the CD4 binding sight. Another suitable antibody to make a conjugate is one that binds to an HIV envelope glycoprotein at the gp120-gp41 interface. Such antibodies including, without limitation, an antibody selected from 8ANC195, 35022, and PGT151 described in detail in U.S. Publication No. 20150361160, U.S. Publication No. 20160022803, and U.S. Publication No. 20150152167. Another suitable antibody to make a conjugate is one that binds to the gp41 membrane-proximal external region (MPER) including, without limitation, 4E10, 10E8, 2F5 and Z13e1, described in detail U.S. Publication No. 20160009789, PCT Published Application No. WO2013070776, U.S. Publication No. 20150158934, U.S. Publication No. 20120269821. For making of further antibody conjugates, exemplary antibodies employed in binding to the HIV envelope glycoprotein include without limitation VRC01, VRC07, VRC07-523, 3BNC1 17, NIH45-46, PGV04, b12, CH31 , and CH103. An example of VRC07-523 is set forth in J. Virol, 88(21): pp. 12669-12682 (November 2014). An example of 3BNC1 17 is set forth in U.S. Publication No. 20140212458. An example of NIH45-46 is set forth in U.S. Publication No. 20150274813. An example of PGV04 is set forth in U.S. Publication No. 20130251726. An example of b12 is set forth in U.S. Publication No. 20160009789. An example of CH31 is set forth in U.S. Publication No. 20130251726. An example of CH103 is set forth in U.S. Publication No. 20140212458.

For making of further antibody conjugates, exemplary antibodies employed in binding to the HIV envelope glycoprotein include without limitation of 2G12, 2F5, 3BC176, 3BNC60, 3BNC1 17, 4E10, 8ANC131 , 8ANC195, 10E8, 10-1074, 12Al2, 35022, b12, B2530, CHO1 -04, CH103, CH31 , HJ16, M66.6, N6, N6-LS, NIH45-46, PG9, PG16, PGDM1400, PGT121 , PGT128, PGT135, PGT141-PGT145, PGT151 , PGV04, VRC01 , VRC01-LS, VRC07, VRC07-523, VRC07-LS, and Z13, and preferably VRC01 , VRC01-LS, N6, N6-LS, VRCO7 and VRC07-523, described in detail in WO201210657, WO2016196975, U.S. Pat. No. 8,637,036, US Patent Publication No. 2014/0322163,WO 2016196975 and WO2017/79479, all incorporated by reference herein.

In another aspect, antibody-drug conjugates are conjugated to make anti-axl antibody PSA conjugates. In these embodiments, an anti-axl antibody is one that binds an epitope on the extracellular part of the AXL protein, which is also referred to as UFO or JTK11, a 894 amino acid protein with a molecular weight of 104-140 kDa that is part of the subfamily of mammalian TAM Receptor Tyrosine Kinases (RTKs). These antibodies and particular methods of making these compounds are described in detail in PCT entitled ‘New dosage regimens for antibody drug conjugates based on anti-axl antibodies’ published as WO2018007592, which is incorporated by reference herein.

In another aspect, protein-PSA conjugates are made to a number of cell surface markers and their ligands. For example cancer cells have been reported to express at least one of the following cell surface markers and or ligands, including but not limited to, carbonic anhydrase IX, alpha-fetoprotein, alpha-actinin-4, A3 (antigen specific for A33 antibody), ART-4, B7, Ba-733, BAGE, BrE3-antigen, CA125, CAMEL, CAP-1, CASP-8/m, CCCL19, CCCL21, CD1, CDla, CD2, CD3, CD4, CDS, CD8, CD1-1A, CD14, CD15, CD16, CD18, CD19, CD20, CD21, CD22, CD23, CD25, CD29, CD30,CD32b, CD33, CD37, CD38, CD40, CD4OL, CD45, CD46, CD54, CD55, CD59, CD64, CD66a-e, CD67, CD70, CD74, CD79a, CD80, CD83, CD95, CD126, CD133, CD138, CD147, CD154, CDC27, CDK-4/m, CDK 2A, CXCR4, CXCR7, CXCL12, HIF-1-alpha, colon-specific antigen-p (CSAp), CEA (CEACAMS), CEACAM6, c-met, DAM, EGFR, EGFRvIII, EGP-1, EGP-2, ELF2-M, Ep-CAM,Flt-1, Flt-3, folate receptor, G250 antigen, GAGE, GROB, HLA-DR, HM1.24, human chorionic gonadotropin (HCG) and its subunits, HER2/neu, HMGB-1, hypoxia inducible factor (HIF-1), HSP70-2M, HST-2or 1a, IGF-1R, IFN-gamma, IFN-alpha, IFN-β, IL-2, IL-4R, IL-6R, IL-13R, IL-15R, IL-17R, IL-18R, IL-6, IL-8, IL-12, IL-15, IL-17, IL-18, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5, MUM-1/2, MUM-3, NCA66, NCA95, NCA90, pancreatic cancer mucin, placental growth factor, p53, PLAGL2, prostatic acid phosphatase, PSA, PRAME, PSMA, P1GF, ILGF, ILGF-1R, IL-6, IL-25, RS5, RANTES, T101, SAGE, 5100, survivin, survivin-2B, TAC, TAG-72, tenascin, TRAIL receptors, TNF-alpha, Tn-antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, cMET, an oncogene marker and an oncogene product (see, e.g., Sensi, et al, Clin. Cancer Res. 12 (2006) 5023-5032; Parmiani, et al, J. Immunol. 178 (2007) 1975-1979; Novellino, et al, Cancer Immunol. Immunother. 54 (2005) 187-207). The conjugates obtained with the method as reported herein can be used in the preparation of medicaments for the treatment of e.g. an oncologic disease, a cardiovascular disease, an infectious disease, an inflammatory disease, an autoimmune disease, a metabolic (e.g., endocrine) disease, or a neurological (e.g. neurodegenerative) disease. Exemplary non-limiting examples of these diseases are Alzheimer's disease, non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom's macroglobulinemia, carcinomas (such as carcinomas of the oral cavity, gastrointestinal tract, colon, stomach, pulmonary tract, lung, breast, ovary, prostate, uterus, endometrium, cervix, urinary bladder, pancreas, bone, liver, gall bladder, kidney, skin, and testes), melanomas, sarcomas, gliomas, and skin cancers, acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis, or fibrosing alveolitis. These proteins are described in detail in PCT entitled ‘Novel methods for enzyme mediated polypeptide conjugation using sortase’ published as WO2016096741, which is incorporated by reference herein.

In another aspect, anti-mesothelin antibody-PSA conjugates are made using antibodies reactive to mesothelin to conjugate them with PSA. In these embodiments, it is preferred that the antibody used to make a PSA-conjugate recognize an epitope of mesothelin that is not masked by another extracellular antigen. In another aspect of these embodiments, the antibody-PSA conjugates are used in methods of treatment of cancer such as pancreatic, ovarian, mesothelioma and lung cancer. These proteins are described in detail in US entitled ‘Anti-mesothelin antibodies and uses thereof’ published as US 20150259433, which is incorporated by reference herein.

In another aspect, enzymes are conjugated to make enzyme PSA conjugates. In these embodiments, suitable enzymes to be used to prepare PSA conjugates include without limitation an enzyme from the esterase group comprising a metalloproteinase, a subtilase, or a lipase, triacylglycerol lipase, subtilase, metalloproteinase, cholinesterase, acetylcholinesterase, butyrylcholinesterase, trypsin, subtilisin, thermolysin, or CT, cholinesterase, acetylcholinesterase, butyrylcholinesterase, subtilase, subtilisin, thermolv sin, lipase, triacylglycerol lipase, metalloproteinase, chymotrypsin, -chymotrypsin, or trypsin, an enzyme polymer conjugate such as an esterase-polymer conjugate comprising a chymotrypsin-pDMAEMA (CT-pDMAEMA) conjugate, a metalloproteinase-pOEGMA conjugate, a thermolysin-pOEGMA conjugate, a subtilisin-ionic liquid polymer conjugate, a subtilase-ionic liquid polymer conjugate, or a lipase-pDMAA conjugate. In these embodiments, the chemical reaction catalyzed by the enzyme-polymer conjugate may include, for example, a transesterification reaction, a hydrolysis reaction, an enantioselective reaction, a redox reaction, a condensation reaction, a polyester synthesis reaction, or a peptide synthesis reaction, or combinations of any thereof. These proteins are described in detail in PCT application entitled ‘Non-aqueous enzyme-polymer conjugate solutions and related methods’ published as WO 2016130677, which is incorporated by reference herein.

In another aspect, ligand-drug conjugates are conjugated to make ligand-drug-PSA conjugates. In these embodiments, novel PSA conjugates are made from antibody-drug conjugates. These embodiments include the making and use of PSA antibody conjugates where the antibodies are modified to be linked to a drug, such as a cytotoxic agent. Specific antibodies that can be used for the antibody-drug conjugates of the present invention include, but are not limited to, anti-HER2 monoclonal antibody such as trastuzumab and pertuzumab, anti-C820 monoclonal antibody such as rituximab, ofatumumab, tositumomab and ibritumomab, anti-CA125 monoclonal antibody such as oregovomab, anti-EpCAM (17-1A) monoclonal antibody such as edrecolomab, anti-EGER monoclonal antibody such as cetuximab, partitumunab and nimotuzumab, anti-CD 30 monoclonal antibody such brentuximab, anti-CD33 monoclonal antibody such as gemtuzumab and huMy9-6, anti-vascular integrin alpha-v β-3 monoclonal antibody such as etaracizmab, anti-CD52 monoclonal antibody such as 5 alemtuzumab, anti-CD22 monoclonal antibody such as epratuzumab, anti-CEA monoclonal antibody such as labetuzumab, anti-CD44v6 monoclonal antibody such as bivatuzumab, anti-FAP monoclonal antibody such as sibrotuzumab, anti-CD19 monoclonal antibody such as huB4, anti-CanAg monoclonal antibody such as huC242, anti-CD56 monoclonal antibody such huN901, anti-CD38 monoclonal antibody such as darattunumab, anti-CA6 monoclonal antibody 10 such as DS6, anti-IGF-IR monoclonal antibody such as cixutumumab and 3B7, anti-integrin monoclonal antibody such as CNTO 95, and anti-syndecan-1 monoclonal antibody such as B-B4. In tertian embodiments, binding proteins other than antibodies can also be used as the cell-binding ligand for the ligand-drug conjugates including ut not limited to interferons such as IFN-α, IFN-f3, and IFN-y, transferrins, epidermal growth factors (EGF) and EGF-like domains, gastrin-releasing peptides (GRP), platelet-derived growth factors (PDGF), transforming growth factors (TGF), vaccinia growth factor (VGF), insulin and insulin-like growth factors (IGF) such as IGF-1 and IGF-2, other suitable hormones such as thyrotropin releasing hormones (TRH), melanocyte-stimulating 30 hormones (MSH), steroid hormones (for example, estrogen and androgen), and somatostatin, lymphokines such as IL-2, IL-3, IL-4, and IL-6, colony-stimulating factors (CSF) such as G-CSF, M-CSF and GM-CSF, bombesin, gastrin, and folic acid. These antibody or binding protein conjugates used to make PSA conjugates are described in detail in PCT application entitled ‘Novel hydrophilic linkers and ligand-drug conjugates thereof’ published as WO2016147031, which is incorporated by reference herein.

In another aspect, antibody-conjugates are conjugated to make antibody conjugates that are further conjugated with PSA to provide novel antibody conjugates. In embodiments according to this aspect, yypical examples of antibodies used to make PSA conjugates include, but are not limited to abciximab, rituximab, basiliximab, paliviziiinab, rn ixiniab. trastuzumab, aleintuzumab, adalimumab, tositumoinab-1131, cetuximab, ibrituximab tiuxetan, oirializtunab, bcvacizumab, natalizumab, ranibizumab, panituramab, eculinumab, certolizumab pegol, golinitintab, canakinumab, cattimaxontab, ustekinumab, tocilizurmab, ofatuinumab, denosumab, belimumab, ipilimumab and brentliximab. These antibody or binding protein conjugates used to make PSA conjugates are described in detail in PCT application entitled ‘Antibody-conjugates with improved therapeutic index for targeting cd30 tumours and method for improving therapeutic index of antibody-conjugates’ published as WO2017137457, which is incorporated by reference herein.

In another aspect, antibody-rifamycin conjugates are further conjugated with PSA to provide novel antibody conjugates. These antibody or binding protein conjugates used to make PSA conjugates are described in detail in PCT application entitled ‘Process for the preparation of an antibody-rifamycin conjugate’ published as WO2017152083, which is incorporated by reference herein.

In certain embodiments, antibodies or antigen binding portions thereof reactive to the T cell Ig- and mucin-domain-containing molecule-3 (Tim-3) are conjugated with PSA to form anti-TIM3 conjugates. Suitable antibodies to the mucin-domain-containing molecule-3 (Tim-3) are described in US2018016336, incorporated by reference herein. In another aspect, anti-TIM3/PSA antibody conjugates are used in methods of treatment. In one embodiment, an anti-TIM3/PSA antibody conjugate binds to human T-cell immunoglobulin and mucin-domain containing-3 (TIM3) and binds to soluble human TIM3, binds to membrane bound human TIM3, and induces or enhances T cell activation. In another aspect, antibody conjugates that are made are used in methods for inhibiting the growth of tumors and cancers that include bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer.

In certain embodiments, antibodies or antigen binding portions thereof reactive to the protein Thrombospondin Type-1 Domain-containing 7A (THSD7A) to form anti-THSD7A conjugates. Suitable antibodies to the THSD7A protein are described in WO2017167770, incorporated by reference herein. In another aspect, antibody conjugates that are made are used in methods for inhibiting the growth of tumors and cancers that include kidney, prostate, thyroid, bladder, esophagus, colon and breast cancer.

In another aspect, peptides and peptidomimetics are conjugated with PSA to make peptide-PSA conjugates. In certain embodiments, the peptides and peptidomimetics described in WO2018015296, incorporated by reference herein, are conjugated with PSA to make particular peptide-PSA conjugates. A particular protein used to make conjugates is soluble amyloid precursor protein a (sAPPa). This protein has a particular binding site which allows for binding to the GABABRIa receptor, thereby causing an agonistic effect through specific binding to sushi domain 1 of GABABRIa. Provided herein are soluble amyloid precursor protein a (sAPPa) conjugated with PSA that are able to activate this receptor.

In another aspect, proteins, polypeptides and peptides which are involved in regulatory and signal transduction pathways such as receptors, ligands, second messengers and related proteins and compounds are conjugated to make protein-PSA conjugates. In certain embodiments, the EI24 protein or a fragment thereof is conjugated with PSA to make E124 protein-PSA conjugates. In making a E124-PSA conjugate, the full length polypeptide, fragments or variants of the human E124 protein according to SEQ ID NO:1 (Met-Ala-Asp-Ser-Val-Lys-Thr-Phe-Leu-Gln-Asp-Leu-Ala-Arg-Gly-Ile-Lys-Asp-Ser-Ile-Trp-Gly-Ile-Cys-Thr-Ile-Ser-Lys-Leu-Asp-Ala-Arg-Ile-Gln-Gln-Lys-Arg-Glu-Glu-Gln-Arg-Arg-Arg-Arg-Ala-Ser-Ser-Val-Leu-Ala-Gln- Arg-Arg-Ala-Gln-Ser-Ile-Glu-Arg-Lys-Gln-Glu-Ser-Glu-Pro-Arg-Ile-Val-Ser-Arg-Ile-Phe-Gln-Cys-Cys-Ala-Trp-Asn-Gly-Gly-Val-Phe-Trp-Phe-Ser-Leu-Leu-Leu-Phe-Tyr-Arg-Val-Phe-Ile-Pro-Val-Leu-Gln-Ser-Val-Thr-Ala-Arg-Ile-Ile-Gly-Asp-Pro- Ser-Leu-His-Gly-Asp-Val-Trp-Ser-Trp-Leu-Glu-Phe-Phe-Leu-Thr-Ser-Ile-Phe-Ser-Ala-Leu-Trp-Val-Leu-Pro-Leu-Phe-Val-Leu-Ser-Lys-Val-Val-Asn-Ala-Ile-Trp-Phe-Gln-Asp-Ile-Ala-Asp-Leu-Ala-Phe-Glu-Val-Ser-Gly-Arg-Lys-Pro-His-Pro-Phe-Pro- Ser-Val-Ser-Lys-Ile-Ile-Ala-Asp-Met-Leu-Phe-Asn-Leu-Leu-Leu-Gln-Ala-Leu-Phe-Leu-Ile-Gln-Gly-Met-Phe-Val-Ser-Leu-Phe-Pro-Ile-His-Leu-Val-Gly-Gln-Leu-Val-Ser-Leu-Leu-His-Met-Ser-Leu-Leu-Tyr-Ser-Leu-Tyr-Cys-Phe-Glu-Tyr-Arg-Trp-Phe- Asn-Lys-Gly-Ile-Glu-Met-His-Gln-Arg-Leu-Ser-Asn-Ile-Glu-Arg-Asn-Trp-Pro-Tyr-Tyr-Phe-Gly-Phe-Gly-Leu-Pro-Leu-Ala-Phe-Leu-Thr-Ala-Met-Gln-Ser-Ser-Tyr-Ile-Ile-Ser-Gly-Cys-Leu-Phe-Ser-Ile-Leu-Phe-Pro-Leu-Phe-Ile-Ile-Ser-Ala-Asn-Glu- Ala-Lys-Thr-Pro-Gly-Lys-Ala-Tyr-Leu-Phe-Gln-Leu-Arg-Leu-Phe-Ser-Leu-Val-Val-Phe-Leu-Ser-Asn-Arg-Leu-Phe-His-Lys-Thr-Val-Tyr- Leu-Gln-Ser-Ala-Leu-Ser-Ser-Ser-Thr-Ser-Ala-Glu-Lys-Phe-Pro-Ser-Pro-His-Pro-Ser-Pro-Ala-Lys-Leu-Lys-Ala-Thr-Ala-Gly-HIS, may be utilized. In another aspect, the E124-PSA conjugates are used in methods of treating cancer or the like. Particular but non-limiting types of cancer that are treated in certain embodiments are a cancer selected from the group consisting of breast cancer, cervical cancer, leukemia, gastric cancer, sarcoma, liver cancer, lung cancer, colorectal cancer, and renal carcinoma. The EI24 protein is described in further detail in US20180028605, incorporated by reference herein.

In another aspect, proteins and peptides which are involved in the regulation of the immune system, allergic reactions, and allergies and related proteins and compounds are conjugated to make protein-PSA conjugates. In some embodiments, peptides derived from the PRU P 3 allergen of PRUNUS PERSICA are conjugated to make protein-PSA conjugates. The amino acid sequence Ala-Ser-Ser-Asn-Gly-lle-Arg-Asn-Val-Asn-Asn-Leu-Ala-Arg-Thr-Pro-Asp-Arg-Gln-Ala-Cys (SEQ ID NO:2) developed from various regions of Pru p 3 protein and variants thereof are utilized to make Pm p 3 protein-PSA conjugates. In another aspect, the Pm p 3 protein-PSA conjugates are used in methods of treating immune disorders including the treatment of allergies. The Pru p 3 protein is described in further detail in WO2017051049, incorporated by reference herein.

In another aspect, proteins and peptides which are capable of binding to tumor necrosis factor receptor (TNF) and related proteins and compounds are conjugated to make TNF binding protein-PSA conjugates. In certain embodiments these proteins capable of binding to tumor necrosis factor receptor (TNF) are receptors or binding molecules which comprise an amino acid sequence according to SEQ ID NO:3 (V-P-A-Q-W-F-P-R-S-I-P-E-P-S-N-L-C-Q-P-R-E-Y-Y-D-E-R-A-Q-R-R-C-S-Q-C-P-P-G-C-R-A-K-S-F-C-N-E-T-S-D-T-V-C-V-P-C-E-D-S-T-Y-T-Q-L-W-N-W-L-P-E-C-L-S-C-G-S-R-C-S-T-G-Q-V-E-T-Q-A-C-T-L-K-Q-N-R-I-C-T-C-E-P-G-R-Y-C-I-L-P-R- Q-E-G-C-Q-V-C-G-L-L-R-K-C-P-P-G-F-G-V-A-K-P-G-T-A-T-S-N-W-C-A) or variants thereof. This represents the truncated extracellular domain of equine TNFR p80 polypeptide comprising the CDR2 and CDR3 TNF-binding domains, as predicted from the CDR2 and CDR3 binding domains of the human TNFR p80 isoform, as described by Mukai et al. in Science Signaling 3(148):ra83). In another aspect TNF binding protein-PSA conjugates are used in methods of treating allergies or immune disorders. In particular embodiments, the immune disorder treated by use of a TNF binding protein-PSA conjugate is a condition mediated by TNF that includes an inflammatory mediated condition, a chronic inflammatory disease, arthritis, such as immune mediated polyarthritis, rheumatoid arthritis, osteoarthritis, polyarthritidies, juvenile idiopathic arthritis, psoriatic arthritis, ankylosing spondylitis, Crohn's disease, ulcerative colitis, psoriasis, systemic vasculitis, atopic dermatitis, congestive heart failure, refractory uveitis, bronchial asthma, allergic conditions, sepsis, shock, diabetes mellitus, and neuro-degenerative conditions, such as Alzheimer's disease, Parkinson's disease, stroke and amyotrophic lateral sclerosis. The TNF binding proteins which are conjugated with PSA are described in further detail in WO2016119023, incorporated by reference herein.

In another aspect, proteins and peptides which are capable of binding to the erythropoietin receptor (EPOR) and related proteins and compounds are conjugated to make EPOE protein-PSA conjugates. In certain embodiments, EPOE protein-PSA conjugates provided herein are used as to block or inhibit a EPO/EPOR signaling pathway and in particular in methods of treating cancer, and in particular hepatocellular carcinoma. The EPOR proteins which are conjugated with PSA are described in further detail in WO2017219951, incorporated by reference herein.

In another aspect, non-protein compounds are conjugated with PSA to form further novel compounds conjugated to PSA. In another aspect, these non-protein compound PSA conjugates can be combined with particular protein or peptide-PSA conjugates, for example, in particular formulations or methods of treatment provided herein.

In another group of embodiments, compounds capable of inhibiting the intracellular expression of the p38 gamma protein are conjugated to make p38 inhibitor PSA conjugates. These compounds may utilize protein or peptide PSA conjugates in combination with other non-protein compound PSA conjugates in methods of treatment, including the treatment of cancer, in certain embodiments. One particular type of non-limiting cancer for which these embodiments are created and particularly effective is for the treatment of hepatic cancer.

In certain embodiments the compounds which are capable of inhibiting the intracellular expression of the p38 gamma protein are peptides which comprise an amino acid sequence according to SEQ ID NO:4 (Y-G-R-K-K-R-R-Q-R-R-R-A-R-V-P-K-E-T-A-L). The compounds may be combined with other compounds, which themselves may or may not be novel, in formulations for the treatment of cancer and other diseases. For example, the above peptide is used to form a PSA conjugate that is used in formulations that optionally include other compounds that may or may not be PSA conjugates depending on the particular embodiment. Particular compounds suitable for making novel PSA conjugates are described in a PCT patent application published as WO2016198698, incorporated by reference herein.

In another aspect, the Clar Cell 10 kDa protein termed CCIO is conjugated to make CCIO protein-PSA conjugates. In certain embodiments, CCIO protein-PSA conjugates provided herein are used in methods of treating viral infections, and in particular influenza infection, including Type A influenza, H1N1 influenza, and ebola virus disease (EVD) also known as ebola hemorrhagic fever (EHF). The CCIO proteins which are conjugated with PSA are described in further detail a PCT patent application published as WO2016133560, incorporated by reference herein.

In another aspect, the soluble recombinant protein M1 type Kunin polypeptide is conjugated to make Kunin protein-PSA conjugates. In certain embodiments according to this aspect, a soluble recombinant P-selectin protein is used to form a novel Kunin protein PSA conjugate. In another aspect, these novel Kunin protein PSA conjugate are used in formulations and methods of treatment, including in particular for the treatment of endometriosis. The soluble recombinant protein M1 type Kunin polypeptide which is conjugated with PSA are described in further detail a Chinese patent application by Guo, Sunwei et al., published as CN106146669, incorporated by reference herein.

In another aspect, a Bombus terrestris larva-derived polypeptide is conjugated to make Bombus terrestris protein-PSA conjugates. In another aspect, these novel Bombus terrestris protein-PSA conjugates are used in formulations and methods of treatment, including in particular for the treatment and or prevention of erectile dysfunction and arteriosclerosis. The Bombus terrestris larva-derived polypeptide which is conjugated with PSA are described in further detail in a Korean patent application by Ahn Mi Young AC et al., published as KR20150073932, incorporated by reference herein.

In another aspect, the junctional adhesion molecule protein termed JAMA-A is conjugated to make JAMA-A protein-PSA conjugates. The novel JAMA-A PSA conjugates are used in methods of treating immune disorders. In certain embodiments, JAMA-A protein-PSA conjugates provided herein are used in methods of treating arthritis, and in particular rheumatoid arthritis (RA). The JAMA-A protein which is conjugated with PSA are described in further detail in a Chinese patent application by Li Meizhang, et al. published as CN105125548, incorporated by reference herein.

In another aspect, the tumor necrosis factor-alpha induced protein 8 like-2 termed TIPE2 is conjugated to make TIPE2 protein-PSA conjugates. The novel TIPE2 PSA conjugates can be used in methods of immunoregulation or treating immune disorders. In certain embodiments, TIPE2 protein-PSA conjugates provided herein are used in methods of treating endotoxin sepsis complicated with acute lung injury. The TIPE2 protein which is conjugated with PSA are described in further detail in a Chinese patent application by Tao Zhen Gang, et al. published as CN105126106, incorporated by reference herein.

In another aspect, mucus-penetrating peptides are conjugated to PSA make mucus-penetrating peptide PSA conjugates which can be utilized in conjunction with other compounds to enhance penetration and delivery of therapeutic compounds through intranasal delivery where mucosal penetration is enhanced. In these embodiments, typically peptides of between five to ten amino acids can be used to form PSA conjugates, and in many embodiments the peptides are about six to eight amino acids in length. The novel mucus-penetrating peptide PSA conjugates can be used in a wide variety of methods treatment where an intra nasal delivery is desired. The mucus-penetrating peptides which are conjugated with PSA are described in further detail in a PCT patent application published as WO2018013907, incorporated by reference herein.

In another aspect, glucagon-receptor selective analogs and peptide derivatives thereof are conjugated to PSA to make novel glucagon receptor analog peptide PSA conjugates that have enhanced therapeutic properties. In these embodiments, typically peptides of between five to fifteen amino acids can be used to form PSA conjugates, and in many embodiments the peptides are about seven to ten amino acids in length, and in many embodiments, peptides having nine amino acids are used to form novel PSA conjugates. In another aspect, novel glucagon receptor analog peptide PSA conjugates provided herein are used in methods of treatment of metabolic disorders including in particular methods of treating obesity and diabetes. The particular glucagon-receptor selective analogs and peptide derivatives which are conjugated with PSA are described in further detail in a US patent application published as US2018009871, incorporated by reference herein.

In another aspect, peptide-based therapeutic agents are conjugated to PSA to make novel peptide-based therapeutic agents PSA conjugates that have enhanced therapeutic properties. Non-limiting examples of peptides that are used in these embodiments include SEQ ID NO:5 (Arg-Pro-Met-Arg-Leu-Glu-Ser-Phe-Ser-Ala-Cys-Ile-Trp-Val-Lys-Ala-Thr-Asp-Val-Leu-Asn-Lys-Thr-Ile-Leu-Phe-Ser-Tyr-Gly-Thr-Lys-Arg-Asn-Pro-Tyr-Glu- Ile), and SEQ ID NO:6 (Gly-Gly-Gly-Phe-Asp-Glu-Thr-Leu-Ala-Phe-Ser-Gly-Arg-Leu-Thr-Gly-Phe-Asn-Ile-Trp-Asp-Ser-Val-Leu-Ser-Asn-Glu-Glu-Ile-Arg-Glu-Thr-Gly-Gly-Ala-Glu-Ser-Cys-His-Ile). In another aspect, novel peptide-based therapeutic agents conjugated to PSA are used in methods of treating cancer, in particular embodiments breast cancer, lung cancer, nasopharyngeal cancer, and epithelial cancer. The particular peptide-based therapeutic agents which are conjugated with PSA are described in further detail in a US patent application published as US20180002400, incorporated by reference herein.

In another aspect, antibodies that bind to a Tau protein epitope are conjugated to PSA to make novel anti-Tau antibody PSA conjugates that have enhanced therapeutic properties. In certain embodiments of this aspect, novel PSA conjugated antibodies that selectively recognize a pathological type Tau protein. In particular embodiments, particular epitopes that confer a higher affinity for pathological type Tau protein as compared to normal Tau include amino acid sequences occurring within a pathological type Tau protein selected from the following: SEQ ID NO:7 (K-H-Q-P-G-G-G), SEQ ID NO:8 (K-H-V-P-G-G-G), SEQ ID NO:9 (H-H-K-P-G-G-G), and SEQ ID NO:10 (T-H-V-P-G-G-G). In another aspect, the novel anti-Tau antibody PSA conjugates provided here are used in methods of treating and preventing Alzheimer's disease and other forms of dementia. The particular antibodies which are conjugated with PSA and preferred epitopes are further described in Australian patent application published as AU2017272259, incorporated by reference herein.

In another aspect, alpha-helix antibacterial peptide GV's are conjugated to PSA to make novel alpha-helix antibacterial peptide PSA conjugates that have enhanced antibacterial or other therapeutic properties. In another aspect, the novel antibacterial peptide conjugates provided herein are used in methods of treatment, in particular for the treatment of bacterial infections. The particular alpha-helix antibacterial peptide GV peptides which are conjugated with PSA are further described in Chinese patent application published as CN106366162, incorporated by reference herein.

In another aspect, polypeptides for modifying microbubbles and GBM-targeting (glioblastoma multiforme targeting) are conjugated to PSA to make novel polypeptide PSA conjugates that have enhanced therapeutic properties. In a particular embodiment of this aspect, a suitable polypeptide for modifying microbubble comprises a lipophilic domain, a cell killing domain and a tumor targeting domain arranged in order from the N-terminal to the C-terminal, wherein the tumor targeting domain is a low-density A lipoprotein peptidomimetic peptide, linked between a lipophilic domain and a cell killing domain and between a cell killing domain and a tumor targeting domain, is connected by a flexible connecting peptide. The particular polypeptides for modifying microbubbles and GBM-targeting which are conjugated with PSA are further described in Chinese Patent application by Ren Jinghua et al., published as CN106632688, incorporated by reference herein.

In another aspect, PEDF-derived peptide/polypeptides for promoting muscle or tendon regeneration or arteriogenesis are conjugated to PSA to make novel PEDF-derived polypeptides PSA conjugates that have enhanced therapeutic properties. The PEDF-derived peptide/polypeptides for promoting muscle or tendon regeneration can be synthetic, and they can be various lengths including at least 20 amino acids in length, about 29 to 30 amino acids in length, or alternatively having more or few amino acids such that they are typically, but not always, five to fifty amino acids in length. The particular PEDF-derived polypeptides for promoting muscle or tendon regeneration or arteriogenesis are described in detail in Japanese Patent application by Tsao Yeou-Ping et al., published as JP2017165745, incorporated by reference herein.

In another aspect, peptides for promoting the activity of the SERCA calcium pump are conjugated to PSA to make novel SERCA calcium pump activation peptide PSA conjugates that have enhanced therapeutic properties. Suitable peptides for these embodiments include the following: M-A-E-K-E-S-T-S-P-H-L-M-V-P-I-L-L-L-V-G-W-I-V-G-C-I-I-V-I-Y-I-V-F-F (SEQ ID NO:11), M-A-E-K-A-E-S-T-S-P-H-L-M-V-P-I-L-L-L-V-G-W-I-V-G-C-I-I-V-I-Y-I-V-F-F (SEQ ID NO:12), M-A-E-K-E-S-T-S-P-H-L-I-V-P-I-L-L-L-V-G-W-I-V-G-C-I-I-V-I-Y-I-V-F-F (SEQ ID NO:13), and M-A-E-K-A-E-S-T-S-P-H-L-I-V-P-I-L-L-L-V-G-W-I-V-G-C-I-I-V-I-Y-I-V-F-F (SEQ ID NO:14). In another aspect, novel PSA conjugates of peptides for promoting the activity of the SERCA calcium pump provided herein are used in methods of treatment, in particular in methods of treating heart and cardiovascular conditions. The particular peptides for promoting the activity of the SERCA calcium pump are described in detail in US Patent application published as US20170298107A1, incorporated by reference herein.

In another aspect, therapeutic peptides for excitatory neurotoxicity related injuries are conjugated to PSA to make novel neurotherapeutic peptide PSA conjugates that have enhanced neurotherapeutic properties, including for the treatment of central nervous system injuries. One particular peptide used in the preparation of novel PSA conjugates has an amino acid sequence of Y-E-K-L-L-D-T-E-I (SEQ ID NO:15) or a functional variant thereof. Peptides useful for excitatory neurotoxicity related injuries that are used in the preparation of novel conjugates are described in detail in greater detail in PCT application published as WO2017185249, incorporated by reference herein.

In another aspect, SALL4 Peptides are used to make PSA conjugates. In these embodiments, therapeutic peptides that bind to retinoblastoma binding protein 4 RBBp4 are conjugated with PSA to form novel peptide conjugates that, upon binding, blocks the SALL4-RBBp4 interaction. In another aspect, methods for treating a subject having a disorder mediated by a dysregulation of SALL4 are also provided, including embodiments of methods of using novel peptide PSA conjugated developed and described herein for the treatment of retinoblastoma. Peptides suitable for bind to retinoblastoma binding protein 4 RBBp4 that are used in the preparation of novel conjugates are described in greater detail in PCT application published as WO2017190032, incorporated by reference herein.

In another aspect, peptide conjugates linked with NFKB essential modulator (NEMO) binding domain CARGO sequence are used to make peptide PSA conjugates with enhanced permeability and other therapeutic properties. In another aspect, methods for treating a subject having uveitis or a dry eye disease are also provided. Suitable peptides that are used in the preparation of novel conjugates are described in greater detail in PCT application published as WO2017189826, incorporated by reference herein.

In another aspect, peptide conjugates linked with glucagon like peptide 1 (GLP-1) receptor peptides and analog peptides are used to make peptide PSA conjugates with enhanced therapeutic properties. Peptides having similarity to a GLP-1 having a sequence H-S-Q-G-T-F-T-S-D-Y-S-K-Y-L-D-S-R-R-A-Q-D-F-V-Q-W-L-M-N-T (SEQ ID NO:16) and variants thereof are used to make particular novel GLP-1 peptide-PSA conjugates. In particular embodiments of these peptides, the number 30 or terminal amino acid (C-terminal typically) is replaced with a peptide having an amino acid sequence selected from the group consisting of E-E-P-S-S-G-A-P-P-P-S-OH (SEQ ID NO: 17); E-P-S-S-G-A-P-P-P-S-OH (SEQ ID NO:18); G-A-P-P-P-S-OH (SEQ ID NO:19); G-G-P-S-S-G-A-P-P-P-S-OH (SEQ ID NO:20); G-P-S-S-G-A-P-P-P-S-OH (SEQ ID NO:21); K-R-N-K-N-P-P-P-S-OH (SEQ ID NO:22); K-R-N-K-N-P-P-S-OH (SEQ ID NO:23); K-R-N-K-P-P-I-A-OH (SEQ ID NO:24); K-R-N-K-P-P-P-A-OH (SEQ ID NO:25); K-R-N-K-P-P-P-S-OH (SEQ ID NO:26); K-S-S-G-K-P-P-P-S-OH (SEQ ID NO:27); P-E-S-G-A-P-P-P-S-OH (SEQ ID NO:28); PK-S-G-A-P-P-P-S-OH (SEQ ID NO:29); P-K-S-K-A-P-P-P-S-NH2 (SEQ ID NO:30); P-K-S-K-A-P-P-P-S-OH (SEQ ID NO:31); P-K-S-K-E-P-P-P-S-NH2 (SEQ ID NO:32); P-K-S-K-E-P-P-P-S-OH (SEQ ID NO:33); P-K-S-K-Q-P-P-P-S-OH (SEQ ID NO:34); P-K-S-K-S-P-P-P-S-NH2 (SEQ ID NO:35); P-K-S-K-S-P-P-P-S-OH (SEQ ID NO:36); P-R-N-K-N-N-P-P-S-OH (SEQ ID NO:37); P-S-K-G-A-P-P-P-S-OH (SEQ ID NO:38); P-S-S-G-A-P-P-P-S-E-OH (SEQ ID NO:39); P-S-S-G-A-P-P-P-S-NH2 (SEQ ID NO:40); P-S-S-G-A-P-P-P-S-OH (SEQ ID NO:41); P-S-S-G-A-P-P-P-S-S-OH (SEQ ID NO:42); P-S-S-G-E-P-P-P-S-OH (SEQ ID NO:43); P-S-S-G-K-K-P-P-S-OH (SEQ ID NO:44); P-S-S-G-K-P-P-P-S-NH2 (SEQ ID NO:45); P-S-S-G-K-P-P-P-S-OH (SEQ ID NO:46); P-S-S-G-S-P-P-P-S-OH (SEQ ID NO:47); P-S-S-K-A-P-P-P-S-OH (SEQ ID NO:48); P-S-S-K-E-P-P-P-S-OH (SEQ ID NO:49); P-S-S-K-G-A-P-P-P-S-OH (SEQ ID NO:50); P-S-S-K-Q-P-P-P-S-OH (SEQ ID NO:51); P-S-S-K-S-P-P-P-S-OH (SEQ ID NO:52); S-G-A-P-P-P-S-OH (SEQ ID NO:53); and S-S-G-A-P-P-P-S-OH (SEQ ID NO:54). In another aspect, the novel GLP-1 peptide-PSA conjugates provided herein are used to treat various metabolic disorders, including diabetes. Other suitable peptides that are used in the preparation of novel GLP-1 PSA and related conjugates are described in greater detail in U.S. Pat. Nos. 10,010,614, 9,982,029, 9,839,692, 9,988,430, 7,067,488, and in PCT application published as WO2017200944, all incorporated by reference herein.

In another related aspect, novel GLP-1 and GLP-1 analog polysialyated compounds are provided herein that have improved and desirable formulation and delivery characteristics. In certain embodiments, GLP-1 and GLP-1 analog polysialyated compounds and formulations provided herein have an improved intranasal delivery capability. In another related aspect, novel GLP-1 and GLP-1 analog polysialyated compounds are provided herein that have novel and/or improved therapeutic uses as compared to non-polysialylated GLP-1 and GLP-1 analogs.

In another related aspect, certain embodiments are believed to exhibit a synergistic activity as a result of the effect of polysialic acid moiety's in combination with the effect of polysialyated GLP-1 and GLP-1 analog in particular tissues and cell types. For example, aspect polysialic acid or PSA moieties themselves have been reported to have anti-inflammatory properties in certain neural tissues.

Thus, in other embodiments herein methods of treating neuronal disorders (e.g. neurodegeneration) are provided that use PSA as an anti-inflammatory agent in combination or conjunction with the use of other polysialyated compounds provided herein being useful for the treatment of neuronal disorders or conditions. Exemplary neurode generative diseases that are treated in particular embodiments include amyotropic lateral sclerosis (ALS), Alzheimer's disease, cognitive impairment, demyelinating disease, degenerative retinal diseases, dementia with Lewy bodies, Parkinson's disease, parasomnia, diabetic neuropathy (e.g. retinopathy), or multiple sclerosis. Suitable polysialic acids are described in International Patent Application No. PCT/EP2014/055445, to Newmann rt al., entitled ‘Polysialic acid and use for treatment of neurodegenerative and neuroinflammatory disorders, incorporated by reference herein.

In certain embodiments, for example, polysialyated Extendatide compounds are provided that are useful for the treatment of Parkinson's disease. In one report Extendine-4/Extendatide has been explored as a potential Parkinson's disease therapeutic. See Aviles-Olmo, I., et al., Extendatide and the treatment of patients with Parkinson's disease, J. Clin Invest., 3013, 123(6):2730-2736.doi:10.11723C168295, incorporated by reference herein.

In another aspect, certain embodiments provided herein use formulations consisting essentially of, or consisting of, polysialic acid compounds for use as an anti-inflammatory agent and for the treatment of diseases and disorders associated with inflammation. Thus while some embodiments use polysialic acid covalently linked to therapeutic compound or combination with a therapeutic compound, other embodiment use polysialic acid compounds without other therapeutic compounds.

In another aspect, peptide DNA conjugates useful for treatment of human epidermal growth factor receptor (HER) overexpressing cancers are used to make peptide PSA conjugates with enhanced therapeutic properties. In certain embodiments, a peptide-polynucleotide chimera comprising one or more human epidermal growth factor receptor (HER) binding peptides, a linker, and a single-stranded polynucleotide is used to form a novel PSA conjugates. In another aspect, the novel (HER) binding chimera PSA conjugates are used in methods of treating cancer, in particular in cancers which overexpress the human epidermal growth factor receptor. Other suitable peptides and compounds that are used in the preparation of novel PSA conjugates are described in greater detail in PCT application published as WO2017200787, incorporated by reference herein.

In another aspect, antimicrobial peptide TP4 for treating a cancer are used to make peptide PSA conjugates with enhanced therapeutic properties. A TP4 peptide having the amino acid sequence F-I-H-H-I-I-G-G-L-F-S-A-G-K-A-I-H-R-L-I-R-R-R-R-R (SEQ ID NO:55) or variants thereof are used to make novel peptide PSA conjugates. In another aspect, the TP4 PSA conjugates provided herein are used in compositions and in methods of treating cancer, including for example a malignant, MDR cancer, a recurrent cancer or a metastatic cancer, or a triple negative breast cancer (TNBC). Other suitable peptides and compounds that are used in the preparation of novel PSA conjugates are described in greater detail in Taiwanese application published as TW201725048, incorporated by reference herein.

In another aspect, peptide and mimetic binding agonist of the polio-like kinase 1 polo box domain are used to make peptide PSA conjugates with enhanced therapeutic properties. In another aspect, the polio-like kinase 1 polo box domain PSA conjugates provided herein are used in compositions and in methods of treating cancer, including but not limited to Acute Lymphoblastic Leukemia, Acute Myeloid Leukemia, Adrenocortical Carcinoma, Basal Cell Carcinoma, Bladder Cancer, Bone Cancer, Brain Tumor, Breast Cancer, Cervical Cancer, Chronic Lymphocytic Leukemia, Chronic Myelogenous Leukemia, Chronic Myeloproliferative Disorders, Colon Cancer, Colorectal Cancer, Cutaneous T-Cell Lymphoma, Esophageal Cancer, Ewing Family of Tumors, Retinoblastoma, Gastric (Stomach) Cancer, Gastrointestinal Tumors, Glioma, Head and Neck Cancer, Hepatocellular (Liver) Cancer, Hodgkin Lymphoma, Islet Cell Tumors (Endocrine Pancreas), Kidney (Renal Cell) Cancer, Laryngeal Cancer, Non-small Cell Lung Cancer, Small Cell Lung Cancer, Lymphoma, Medulloblastoma, Melanoma, Pancreatic Cancer, Prostate Cancer, Renal Cancer, Rectal cancer, and Thyroid Cancer. Particular suitable peptides and compounds that are used in the preparation of novel PSA conjugates according to this aspect are described in greater detail in PCT application published as WO2017082924, incorporated by reference herein.

In another aspect, proteins specific for calcitonin gene-related peptide (CGRP) are used to make PSA conjugates with enhanced therapeutic properties. In certain embodiments, hNGAL muteins that bind CGRP are used to make PSA conjugates herein. In another aspect, novel CGRP binding polypeptide PSA conjugates are used in methods of treatment, such as for the treatment of migraine headaches and related conditions. Suitable CGRP binding polypeptide that are used in the preparation of novel PSA conjugates according to this aspect are described in greater detail in Taiwanese application published as TW201725212, incorporated by reference herein.

In another aspect, peptides for the treatment and/or prevention of ischemia and ischemia-reperfusion injury are used to make PSA conjugates with enhanced therapeutic properties. In another aspect, the novel ischemia and ischemia-reperfusion injury peptide PSA conjugates provided herein are used in methods of treatment or ischemia and ischemia-reperfusion injury and related disorders. Particular peptide variants and compounds that are used in the preparation of novel PSA conjugates according to this aspect are described in greater detail in PCT application published as WO2017117381, incorporated by reference herein.

In another aspect, peptides for assisting in the delivery of immunoglobulins across the blood brain barrier are used to form novel PSA peptide conjugates. In certain embodiments according to this aspect, the peptides for assisting in the delivery of immunoglobulins across the blood brain barrier have an immunoglobulin affinity ligand, covalently attached to a linker moiety, covalently attached to a blood-brain barrier agent. A suitable blood brain barrier agent in these embodiments include peptides having the sequence L-R-V-R-L-A-S-H-L-R-K-L-R-K-R-L-L-R-D-A (SEQ ID NO:56), or variants thereof having one or two conservative amino acid substitutions. Particular peptides for assisting in the delivery of immunoglobulins across the blood brain barrier that are used in the preparation of novel PSA conjugates according to this aspect are described in greater detail in US application published as US20170058017A1, incorporated by reference herein.

In another aspect, peptide inhibitors of telomerase translocation are used to make PSA conjugates with enhanced therapeutic properties. In certain embodiments according to this aspect, a suitable peptide inhibitor of telomerase translocation comprises R-R-R-G-G-Xj-A-S-R-S-L-P-L-P-K-R-P-R-R, or variants thereof, where Xi is a phosphomimetic residue selected from the group consisting of as aspartic acid (SEQ ID:57) and glutamic acid (SEQ ID:58). In another aspect of these embodiments, compositions and methods for treating or preventing cardiac or vascular toxicity in a subject receiving a chemotherapeutic agent, where the cardiac or vascular toxicity is associated with administration of the chemotherapeutic agent are provided using novel PSA conjugated with peptide inhibitors of telomerase translocation. Particular peptide inhibitors of telomerase translocation that are used in the preparation of novel PSA conjugates according to this aspect are described in greater detail in PCT application published as WO2017040309, incorporated by reference herein.

In another aspect, compositions and methods relating to cell-penetrating conjugates for the delivery of therapeutic polypeptides or polynucleotides to cells or tissues of the body are used to make PSA conjugates with enhanced therapeutic properties. In certain embodiments according to this aspect, a suitable cell-penetrating delivery conjugates has a cell penetrating peptide, a nuclear localization signal, an effector moiety, and optionally an epitope tag, which together form a compound used to make novel PSA conjugates provided herein. A suitable cell penetrating peptide according to this aspect includes without limitation a HIV TAT protein or a fragment thereof comprising the protein transduction domain, Drosophila Antennapedia (Antp) peptide, and a polyarginine (Arg8) peptide. In another aspect, these novel conjugates are used in compositions and methods for delivering various therapeutic agents. Cell-penetrating conjugates according to this aspect are described in greater detail in PCT application published as WO2017048466, incorporated by reference herein.

In another aspect, polypeptides targeting HIV fusion protein are used to make PSA conjugates with enhanced therapeutic properties. A suitable polypeptide targeting HIV fusion according to this aspect comprises three active domains wherein one domain is an anti-CD4 Adnectin protein, a second domain is a gp41 binding moiety and a third domain is a HIV fusion peptide inhibitor moiety. In another aspect, novel PSA conjugates of the polypeptides targeting HIV fusion protein are used to treat HIV and related viral infections and related disorders. Polypeptide targeting HIV fusion according to this aspect are described in greater detail in PCT application published as WO2016171980, incorporated by reference herein.

In another aspect, peptides that home, distribute to, target, are directed to, accumulate in, migrate to, and/or bind to cancerous cells are used to make novel cancer target peptide PSA conjugates. In another aspect, PSA conjugates of peptides that home, distribute to, target, are directed to, accumulate in, migrate to, and/or bind to cancerous cells are used in methods and formulations for treating cancer and related disorders. In certain embodiments, the peptides of the disclosure can comprise a sequence listed in AU2016283391. Other suitable peptides to use for conjugates are described in detail in Australian Patent Application entitled ‘Therapeutic peptides and methods of use thereof’ and published as AU2016283391 (A1), incorporated herein by reference.

In another aspect, a carboxyl terminal peptide and long-acting interferon is used to make novel PSA conjugates. A suitable new carboxyl terminal peptide has the amino acid sequence S-S-G-S-S-S-S-S-S-S-K-A-P-P-P-S-L-P-S-P-S-R-L-P-G-P-S-D-T-P-I-L-P-Q-N-G-S (SEQ ID NO:59), or a variant thereof. Other suitable peptides to use in making PSA conjugates include those described in Chinese Patent Application published as CN106397570, incorporated herein by reference.

In another aspect, integrin binding peptides are conjugated to PSA to make novel integrin binding peptides PSA conjugates that have enhanced therapeutic properties. In certain embodiments of this aspect, preferred integrin binding peptides have an amino acid sequence VGDLTYLK (SEQ ID NO:60) and VGDLTYLKK (SEQ ID NO:61). In another aspect, the novel integrin binding peptides PSA conjugates provided herein are used in methods of treatment of diseases and disorder, including cancer, an inflammatory disease, an autoimmune disease, chronic fibrosis, chronic obstructive pulmonary disease (COPD), lung emphysema, radiation-induced pulmonary fibrosis, and chronic wounding skin disease. In particular embodiments the novel integrin binding peptides PSA conjugates provided herein are used to treat the following cancers: pancreatic cancer, breast cancer, colorectal cancer, prostate cancer, an oral squamous cell carcinoma. The particular integrin binding peptides which are conjugated with PSA are further described in PCT patent application published as WO2017218569, incorporated by reference herein.

In another aspect, novel PSA conjugates are made using antibodies, binding proteins, or an antigen-binding fragment capable of binding to a muramyl peptide, preferably where said muramyl peptide comprises muramic acid and an amino acid selected from the group consisting of alanine, isoglutamine, glutamic acid, or a salt thereof. In another aspect, novel PSA conjugates of antibodies or the like that bind a muramyl peptide, or a derivative or an analog are used in methods of treatment, including but not limited to treating an autoimmune or inflammatory disease. Other suitable antibodies useful for making PSA conjugates are described in detail in US Patent Application published as US2017342136, incorporated herein by reference.

In another aspect, novel PSA conjugates are made using tumor necrosis factor-related apoptosis-inducing ligand (TRAIL) to make PSA conjugates with enhanced antineoplastic activity, penetration and other therapeutic properties. Suitable peptides and proteins useful for making PSA conjugates are described in detail in US Patent Application published as US2017247427, incorporated herein by reference.

In another aspect, novel PSA conjugates are made using anti-inflammatory peptide separated from haliotis discus hannai abalone visceral organ to make PSA conjugates with therapeutic properties. In a particular aspect, the novel PSA conjugates made can be used in the treatment of inflammatory and immune system diseases and disorders. Suitable peptides useful for making PSA conjugates are described in detail in Chinese Patent Application by Qian Zhongji, published as CN10544010, incorporated herein by reference.

In another aspect, novel PSA conjugates are made using peptide toxins to make novel conjugates with enhanced therapeutic properties or having other desirable characteristics. Suitable peptide toxin for conjugation to PSA to form novel PSA-toxin conjugates include Botulinum toxins (A and B) and conotoxins (e.g. α, δ, κ, μ, ω, Ziconotide, Leconotide, etc.). Suitable Botulinum neurotoxins are described in U.S. Pat. Nos. 7,780,967, 7,758,873, 9,220,783, and 9,598,685, all incorporated herein by reference. Methods for determining Botulinum neurotoxin biological activity are described in US Pat. No. 9,212,355, incorporated herein by reference. Leconotide, reported to be a synthetic ω-conotoxin MVIIA normally found in a fish-eating marine snail, is a particularly suitable conotoxin for making PSA peptide conjugates. Ziconotide is another particularly suitable conotoxin for making PSA peptide conjugates, and it is described in McGivern, J., Neuropsychiatric Dis. Treat., 2007, 3(I), 69-85 and U.S. Pat. No. 7,524,812, all incorporated herein by reference. Suitable Ziconotide peptide sequences for use in making PSA conjugates include SEQ ID NO:62 (C-K-G-K-G-A-K-C-S-R-L-M-Y-D-C-C-T-G-S-C-R-S-G-K-C) and analogs thereof

In another aspect, novel PSA conjugates are made using cannabinoids to make PSA peptide conjugates with therapeutic properties or having other desirable characteristics. Suitable cannabinoids include but are not limited to tetrahydrocannabinol (THC), tetrahydrocannabinolic acid (THCA), cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), cannabicitran (CBT). In some embodiments, the cannabinoid-PSA conjugates provided herein and used in methods of treating a condition, disease, or disorder such as obesity, anorexia, emesis, pain, neuropathic pain, multiple sclerosis, neuroprotection, inflammation, cancer, Parkinson's disease, Huntington's disease, Tourette's syndrome, Alzheimer's disease, epilepsy, bipolar disorder, schizophrenia, post-traumatic stress disorder (PTSD), depression, anxiety, and insomnia. In certain embodiments, PSA-cannabinoids provided herein are formulation in substantially non-aqueous formulations and the like that have water and non-aqueous components (e.g. oils, lipids, fatty acids, and the like), for example, to facilitate solubility.

In other aspects of this embodiment, a PSA-protein conjugate disclosed herein reduces the severity of a disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a PSA-protein conjugate disclosed herein reduces the size of a tumor from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A pharmaceutical composition disclosed herein may comprise a PSA-protein conjugate in an amount sufficient to allow customary administration to an individual. In aspects of this embodiment, a pharmaceutical composition disclosed herein may be, e.g., at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg. at least 200 mg, at least 300 mg, at least 400 mg, at least 500 mg, at least 600 mg, at least 700 mg, at least 800 mg, at least 900 mg, at least 1 g, at least 2 g, at least 3 g, at least 4 g, at least 5 g, at least 6 g, at least 7 g, at least 8 g, at least 9 g, at least 10 g, at least 15 g, at least 20 g, at least 25 g, at least 50 g, at least 100 g of a PSA-protein conjugate.

In other aspects of this embodiment, a pharmaceutical composition disclosed herein may be, e.g., at least 5 mg, at least 10 mg, at least 20 mg, at least 25 mg, at least 50 mg, at least 75 mg, at least 100 mg, at least 200 mg, at least 300 mg, at least 400 mg, at least 500 mg, at least 600 mg, at least 700 mg, at least 800 mg, at least 900 mg, at least 1 g, at least 2 g, at least 3 g, at least 4 g, at least 5 g, at least 6 g, at least 7 g, at least 8 g, at least 9 g, at least 10 g, at least 15 g, at least 20 g, at least 25 g, at least 50 g, at least 100 g of a PSA-protein conjugate. In yet other aspects of this embodiment, a pharmaceutical composition disclosed herein may be in the range of, e.g., about 5 mg to about 100 mg, about 10 mg to about 100 mg, about 50 mg to about 150 mg, about 100 mg to about 250 mg, about 150 mg to about 350 mg, about 250 mg to about 500 mg, about 350 mg to about 600 mg, about 500 mg to about 750 mg, about 600 mg to about 900 mg, about 750 mg to about 1,000 mg, about 850 mg to about 1,200 mg, about 250 mg to about 10 g, about 500 mg to about 7.5 g, about 1 g to about 5 g, about 250 mg to about 2.5 g, about 500 mg to about 2.5 g, or about 1,000 mg to about 1,500 mg. In still other aspects of this embodiment, a pharmaceutical composition disclosed herein may be in the range of, e.g., about 10 mg to about 250 mg, about 10 mg to about 500 mg, about 10 mg to about 750 mg, about 10 mg to about 1,000 mg, about 10 mg to about 1,500 mg, about 50 mg to about 250 mg, about 50 mg to about 500 mg, about 50 mg to about 750 mg, about 50 mg to about 1,000 mg, about 50 mg to about 1,500 mg, about 100 mg to about 250 mg, about 100 mg to about 500 mg, about 100 mg to about 750 mg, about 100 mg to about 1,000 mg, about 100 mg to about 1,500 mg, about 200 mg to about 500 mg, about 200 mg to about 750 mg, about 200 mg to about 1,000 mg, about 200 mg to about 1,500 mg, about 5 mg to about 1,500 mg, about 5 mg to about 1,000 mg, or about 5 mg to about 250 mg.

The final concentration of a PSA-protein conjugate disclosed herein in a pharmaceutical composition disclosed herein may be of any concentration desired. In an aspect of this embodiment, the final concentration of a PSA-protein conjugate in a pharmaceutical composition may be a therapeutically effective amount. In other aspects of this embodiment, the final concentration of a PSA-protein conjugate in a pharmaceutical composition may be, e.g., at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL, at least 500 mg/mL, at least 700 mg/mL, at least 1,000 mg/mL, or at least 1,200 mg/mL. In other aspects of this embodiment, the concentration of a PSA-protein conjugate disclosed herein in the solution may be, e.g., at most 1,000 mg/mL, at most 1,100 mg/mL, at most 1,200 mg/mL, at most 1,300 mg/mL, at most 1,400 mg/mL, at most 1,500 mg/mL, at most 2,000 mg/mL, at most 2,000 mg/mL, or at most 3,000 mg/mL. In other aspects of this embodiment, the final concentration of a PSA-protein conjugate in a pharmaceutical composition may be in a range of, e.g., about 0.00001 mg/mL to about 3,000 mg/mL, about 0.0001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/mL to about 100 mg/mL.

Aspects of the present specification disclose, in part, treating an individual suffering from disease. As used herein, the term “treating,” refers to reducing or eliminating in an individual a clinical symptom of a disease; or delaying or preventing in an individual the onset of a clinical symptom of a disease. For example, the term “treating” can mean reducing a symptom of a condition, or the severity of a disease, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. Those of skill in the art will know the appropriate symptoms or indicators associated with a specific disease and will know how to determine if an individual is a candidate for treatment as disclosed herein with a PSA-protein conjugate.

In aspects of this embodiment, a therapeutically effective amount of a PSA-protein conjugate disclosed herein reduces a symptom associated with a disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a PSA-protein conjugate disclosed herein reduces a symptom associated with a disease by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a PSA-protein conjugate disclosed herein reduces a symptom associated with a disease by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In yet other aspects of this embodiment, a therapeutically effective amount of a PSA-protein conjugate disclosed herein generally is in the range of about 0.001 mg/kg/day to about 100 mg/kg/day. In aspects of this embodiment, an effective amount of a PSA-protein conjugate disclosed herein may be, e.g., at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day. In other aspects of this embodiment, an effective amount of a PSA-protein conjugate disclosed herein may be in the range of, e.g., about 0.001 mg/kg/day to about 10 mg/kg/day, about 0.001 mg/kg/day to about 15 mg/kg/day, about 0.001 mg/kg/day to about 20 mg/kg/day, about 0.001 mg/kg/day to about 25 mg/kg/day, about 0.001 mg/kg/day to about 30 mg/kg/day, about 0.001 mg/kg/day to about 35 mg/kg/day, about 0.001 mg/kg/day to about 40 mg/kg/day, about 0.001 mg/kg/day to about 45 mg/kg/day, about 0.001 mg/kg/day to about 50 mg/kg/day, about 0.001 mg/kg/day to about 75 mg/kg/day, or about 0.001 mg/kg/day to about 100 mg/kg/day. In yet other aspects of this embodiment, an effective amount of a PSA-protein conjugate disclosed herein may be in the range of, e.g., about 0.01 mg/kg/day to about 10 mg/kg/day, about 0.01 mg/kg/day to about 15 mg/kg/day, about 0.01 mg/kg/day to about 20 mg/kg/day, about 0.01 mg/kg/day to about 25 mg/kg/day, about 0.01 mg/kg/day to about 30 mg/kg/day, about 0.01 mg/kg/day to about 35 mg/kg/day, about 0.01 mg/kg/day to about 40 mg/kg/day, about 0.01 mg/kg/day to about 45 mg/kg/day, about 0.01 mg/kg/day to about 50 mg/kg/day, about 0.01 mg/kg/day to about 75 mg/kg/day, or about 0.01 mg/kg/day to about 100 mg/kg/day. In still other aspects of this embodiment, an effective amount of a PSA-protein conjugate disclosed herein may be in the range of, e.g., about 0.1 mg/kg/day to about 10 mg/kg/day, about 0.1 mg/kg/day to about 15 mg/kg/day, about 0.1 mg/kg/day to about 20 mg/kg/day, about 0.1 mg/kg/day to about 25 mg/kg/day, about 0.1 mg/kg/day to about 30 mg/kg/day, about 0.1 mg/kg/day to about 35 mg/kg/day, about 0.1 mg/kg/day to about 40 mg/kg/day, about 0.1 mg/kg/day to about 45 mg/kg/day, about 0.1 mg/kg/day to about 50 mg/kg/day, about 0.1 mg/kg/day to about 75 mg/kg/day, or about 0.1 mg/kg/day to about 100 mg/kg/day.

In other aspects of this embodiment, an effective amount of a PSA-protein conjugate disclosed herein may be in the range of, e.g., about 1 mg/kg/day to about 10 mg/kg/day, about 1 mg/kg/day to about 15 mg/kg/day, about 1 mg/kg/day to about 20 mg/kg/day, about 1 mg/kg/day to about 25 mg/kg/day, about 1 mg/kg/day to about 30 mg/kg/day, about 1 mg/kg/day to about 35 mg/kg/day, about 1 mg/kg/day to about 40 mg/kg/day, about 1 mg/kg/day to about 45 mg/kg/day, about 1 mg/kg/day to about 50 mg/kg/day, about 1 mg/kg/day to about 75 mg/kg/day, or about 1 mg/kg/day to about 100 mg/kg/day. In yet other aspects of this embodiment, an effective amount of a PSA-protein conjugate disclosed herein may be in the range of, e.g., about 5 mg/kg/day to about 10 mg/kg/day, about 5 mg/kg/day to about 15 mg/kg/day, about 5 mg/kg/day to about 20 mg/kg/day, about 5 mg/kg/day to about 25 mg/kg/day, about 5 mg/kg/day to about 30 mg/kg/day, about 5 mg/kg/day to about 35 mg/kg/day, about 5 mg/kg/day to about 40 mg/kg/day, about 5 mg/kg/day to about 45 mg/kg/day, about 5 mg/kg/day to about 50 mg/kg/day, about 5 mg/kg/day to about 75 mg/kg/day, or about 5 mg/kg/day to about 100 mg/kg/day.

In liquid and semi-solid formulations, a concentration of a PSA-protein conjugate disclosed herein typically may be between about 50 mg/mL to about 1,000 mg/mL. In aspects of this embodiment, a therapeutically effective amount of a PSA-protein conjugate disclosed herein may be from, e.g., about 50 mg/mL to about 100 mg/mL, about 50 mg/mL to about 200 mg/mL, about 50 mg/mL to about 300 mg/mL, about 50 mg/mL to about 400 mg/mL, about 50 mg/mL to about 500 mg/mL, about 50 mg/mL to about 600 mg/mL, about 50 mg/mL to about 700 mg/mL, about 50 mg/mL to about 800 mg/mL, about 50 mg/mL to about 900 mg/mL, about 50 mg/mL to about 1,000 mg/mL, about 100 mg/mL to about 200 mg/mL, about 100 mg/mL to about 300 mg/mL, about 100 mg/mL to about 400 mg/mL, about 100 mg/mL to about 500 mg/mL, about 100 mg/mL to about 600 mg/mL, about 100 mg/mL to about 700 mg/mL, about 100 mg/mL to about 800 mg/mL, about 100 mg/mL to about 900 mg/mL, about 100 mg/mL to about 1,000 mg/mL, about 200 mg/mL to about 300 mg/mL, about 200 mg/mL to about 400 mg/mL, about 200 mg/mL to about 500 mg/mL, about 200 mg/mL to about 600 mg/mL, about 200 mg/mL to about 700 mg/mL, about 200 mg/mL to about 800 mg/mL, about 200 mg/mL to about 900 mg/mL, about 200 mg/mL to about 1,000 mg/mL, about 300 mg/mL to about 400 mg/mL, about 300 mg/mL to about 500 mg/mL, about 300 mg/mL to about 600 mg/mL, about 300 mg/mL to about 700 mg/mL, about 300 mg/mL to about 800 mg/mL, about 300 mg/mL to about 900 mg/mL, about 300 mg/mL to about 1,000 mg/mL, about 400 mg/mL to about 500 mg/mL, about 400 mg/mL to about 600 mg/mL, about 400 mg/mL to about 700 mg/mL, about 400 mg/mL to about 800 mg/mL, about 400 mg/mL to about 900 mg/mL, about 400 mg/mL to about 1,000 mg/mL, about 500 mg/mL to about 600 mg/mL, about 500 mg/mL to about 700 mg/mL, about 500 mg/mL to about 800 mg/mL, about 500 mg/mL to about 900 mg/mL, about 500 mg/mL to about 1,000 mg/mL, about 600 mg/mL to about 700 mg/mL, about 600 mg/mL to about 800 mg/mL, about 600 mg/mL to about 900 mg/mL, or about 600 mg/mL to about 1,000 mg/mL.

Dosing can be single dosage or cumulative (serial dosing), and can be readily determined by one skilled in the art. For instance, treatment of a disease may comprise a one-time administration of an effective dose of a pharmaceutical composition disclosed herein. Alternatively, treatment of a disease may comprise multiple administrations of an effective dose of a pharmaceutical composition carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a pharmaceutical composition that includes a PSA-protein conjugate disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition that includes a PSA-protein conjugate disclosed herein that is administered can be adjusted accordingly.

In one embodiment, a PSA-protein conjugate disclosed herein is capable of reducing the severity of a disease in an individual suffering from a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a PSA-protein conjugate is capable of reducing the severity of a disease in an individual suffering from a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, a PSA-protein conjugate and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a PSA-protein conjugate is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a PSA-protein conjugate disclosed herein reduces or maintains the severity of a disease in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a PSA-protein conjugate disclosed herein reduces or maintains the severity of a disease in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a PSA-protein conjugate disclosed herein reduces or maintains the severity of a disease in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A pharmaceutical composition or PSA-protein conjugate is administered to an individual. An individual is typically a human being, but can be an animal, including, but not limited to, dogs, cats, birds, cattle, horses, sheep, goats, reptiles and other animals, whether domesticated or not. Typically, any individual who is a candidate for treatment is a candidate with some form of therapy for a disease the individual is suffering, whether the disease is benign or malignant. With regard to cancer's, the most common types of cancer include, but are not limited to, bladder cancer, breast cancer, colon and rectal cancer, endometrial cancer, kidney cancer, renal cancer, leukemia, lung cancer, melanoma, non-Hodgkins lymphoma, pancreatic cancer, prostate cancer, stomach cancer and thyroid cancer. Pre-operative evaluation typically includes routine history and physical examination in addition to thorough informed consent disclosing all relevant risks and benefits of the procedure.

### Gangliosides

In embodiments, of the invention, gangliosides are conjugated to water soluble polymers, e.g. PEG or PSA or mPSA. Gangliosides are known to provide cells with distinguishing surface markers that can serve in cellular recognition and cell-to-cell communication. They are useful as therapeutic agents.

Conjugates of the invention may comprise a ganglioside and a water soluble polymer, in which the ganglioside comprises a glycosphingolipid (ceramide and oligosaccharide) with one or more sialic acids linked on the sugar chain. Gangliosides can be classified according to how many sialic acid units are present on the molecule. Examples of gangliosides are GM1, GM2 and GM3 (monosialo-gangliosides), GD1a, GD1b, GD2 and GD3 (disialo-gangliosides), GT1b (trisialo-ganglioside) and GQ1 (tetrasialo -ganglio side) .

For use in the present invention, preferred gangliosides comprise a ceramide linked to glucose, which is linked to a first galactose, which is linked to N-acetylgalactosamine, which is linked to a second galactose. This second galactose can be linked to one sialic acid. The first galactose can be linked to one, two, three or four sialic acids. Sialic acids may be linked either as monomers (one on each of the galactose molecules), or as oligosialic acids (2-4 sialic acids) to the first galactose.

Where administered therapeutic gangliosides need to circulate in the blood for long periods. So that their action on target tissues is more effective, gangliosides can be polysialylated, for example, by the method of the invention.

### Drug Delivery Systems

In further embodiments, of the invention, drug delivery systems are conjugated to a water soluble polymer, e.g. PEG or PSA or mPSA. In general, a drug delivery system (DDS) is any molecular or particulate entity which can control the fate and effect of drugs associated with the entity. DDSs can be separated into two general types. The first type comprises macromolecules (MDDSs), for instance antibodies, neoglycoproteins as well as synthetic polymers, such as poly(hydroxypropylmethacrylamide), polylysine and polymerised alkyl cyanoacrylates. The association of drugs with various types of macromolecular carriers, including monoclonal antibodies to target the drug to the desired sites is described for instance by Gregoriadis in Nature 265, 407-411 (1977). The second type is particulate DDSs (PDDSs), which comprises for instance nanospheres or microspheres, which comprise biodegradable materials such as albumin or semibiodegradable materials such as dextran and alkylcyanoacrylate polymers, or vesicles formed of nonionic surfactants or liposomes—for details of which see for example Gregoriadis in NIPS, 4, 146-151 (1989).

Drugs can either be covalently linked to, or passively entrapped into, the DDS. For instance, PDDS comprising surfactant vesicles or liposomes may entrap hydrophilic or hydrophobic pharmaceutically active compounds by being formed of an appropriate combination of layers of surfactant or lipid molecules. Pharmaceutically active compounds are usually covalently linked to MDDSs, by a bond which may or may not be lysed in the body, for instance before or after the active compound performs its function.

Many of the MDDSs have an intrinsic (e.g. antibodies) or acquired (e.g. neoglycoproteins) ability to be recognized by target cells or tissues through receptors on the latter's surface. Typically, such DDSs are taken up specifically by the target upon injection. Specific uptake is, however, limited with the bulk of the DDSs being taken up by other, irrelevant (to therapy) tissues. The reason for this is that antibodies and other DDS proteins (regardless of their specificity for the target) must be, like other proteins, catabolized at the end of their biological life.

Synthetic polymers used in the macromolecular type MDDSs are for instance poly(hydroxypropylmethacrylamide) polylysine and polymerised alkyl cyanoacrylates. These may be catabolised in the reticuloendothelial system (RES) or other tissues by appropriate lysosomal enzymes. It would be desirable to reduce the rate of catabolism of such biodegradable macromolecular type DDS by some means, for instance by reducing uptake of the DDS by the RES or other tissues, or by reducing degradation by lysosomal enzymes once taken up by the RES.

Particulate DDSs (PDDSs) are, as a rule, removed from the circulation by the RES. Because of their propensity for the RES, PDDSs are often used for the delivery of drugs to these tissues. It is often desirable however, that PDDSs are directed to tissues other than those of the RES. To achieve this goal, one must block or delay RES interception of PDDSs.

DDSs for use in the invention may not initially contain glycons. An option is to add or otherwise incorporate a glycon into the DDS structure. Examples of such cases are liposomes incorporating a mannosylated or a galactosylated lipid. These glycoliposomes will target actives to tissues which express a mannose or galactose receptor respectively.

Where DDSs need to circulate in the blood for long periods so that e.g. uptake by target tissues is more effective (as with hepatic parenchymal cells), they are advantageously polysialylated by the methods of the invention.

In an embodiment, a pharmaceutical composition disclosed herein may optionally include a pharmaceutically-acceptable carrier that facilitates processing of an active ingredient into pharmaceutically-acceptable compositions. As used herein, the term “pharmacologically-acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary or excipient.” Such a carrier generally is mixed with an active compound or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, without limitation, water, saline, glycine, hyaluronic acid and the like; solid carriers such as, without limitation, mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7th ed. 1999); REMINGTON: THE SCIENCE AND PRACTICE OF PHARMACY (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10th ed. 2001); and Handbook of Pharmaceutical Excipients (Raymond C. Rowe et al., APhA Publications, 4th edition 2003). These protocols are routine procedures and any modifications are well within the scope of one skilled in the art and from the teaching herein.

In an embodiment, a pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, flavoring agents, coloring agents, and the like. In an embodiment, various buffers and means for adjusting pH can be used to prepare a pharmaceutical composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. In an embodiment, pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. In an embodiment, tonicity adjustors useful in a pharmaceutical composition include, without limitation, salts such as, e.g., sodium chloride, potassium chloride, mannitol or glycerin and other pharmaceutically acceptable tonicity adjustor. In an embodiment, the pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition.

In an embodiment, a therapeutic compound disclosed herein (e.g. a PSA-protein conjugate), or a composition comprising such a therapeutic compound, may be formulated for either local or systemic delivery using topical, enteral or parenteral routes of administration. In an additional embodiment, a therapeutic compound disclosed herein may be formulated by itself in a pharmaceutical composition, or may be formulated together with one or more other therapeutic compounds disclosed herein in a single pharmaceutical composition.

In an embodiment, a therapeutic compound disclosed herein, or a composition formulation such a therapeutic compound, may be made into an inhaled formulation. In an embodiment, inhaled formulations suitable for enteral or parenteral administration include, without limitation, aerosols, dry powders. In an additional embodiment, a therapeutic compound or composition disclosed herein intended for such administration may be prepared according to any method known to the art for the manufacture of pharmaceutical compositions.

In an embodiment, such inhaled dosage forms, the therapeutic compound may be prepared for delivery as an aerosol in a liquid propellant for use in a pressurised (PDI) or other metered dose inhaler (MDI). In an embodiment, propellants suitable for use in a PDI or MDI include, without limitation, CFC-12, HFA-134a, HFA-227, HCFC-22 (difluorochloromethane), HFA-152 (difluoroethane and isobutane). In an embodiment, a therapeutic compound may also be delivered using a nebulisers or other aerosol delivery system. In an embodiment, a therapeutic compound may be prepared for delivery as a dry powder for use in a dry powder inhaler (DPI). In an embodiment, a dry powder for use in the inhalers will usually have a mass median aerodynamic diameter of less than 100 pm, 90 pm, 80 pm, 70 pm, 60 pm 50 pm, 40 pm, 30 pm, 20 pm and 10 pm. In an embodiment, microparticles having aerodynamic diameters in the range of about 5 pm to about 0.5 pm will generally be deposited in the respiratory bronchioles, whereas smaller particles, having aerodynamic diameters in the range of about 2 pm to about 0.05 pm, are likely to be deposited in the alveoli. In an embodiment, a DPI may be a passive delivery mechanism, which relies on the individual's inspiration to introduce the particles into the lungs, or an active delivery mechanism, requiring a mechanism for delivering the powder to the individual. In an embodiment, a therapeutically effective amount of a therapeutic compound disclosed herein for an inhaled formulation may be between about 0.0001% (w/v) to about 90% (w/v), 0.0001% (w/v) to about 80% (w/v), 0.0001% (w/v) to about 70% (w/v), 0.0001% (w/v) to about 60% (w/v), 0.0001% (w/v) to about 50% (w/v), 0.0001% (w/v) to about 40% (w/v), 0.0001% (w/v) to about 30% (w/v), 0.0001% (w/v) to about 20% (w/v), 0.0001% (w/v) to about 10% (w/v), about 0.001% (w/v) to about 90.0% (w/v), 0.001% (w/v) to about 80.0% (w/v), 0.001% (w/v) to about 70.0% (w/v), 0.001% (w/v) to about 60.0% (w/v), 0.001% (w/v) to about 0.0% (w/v), 0.001% (w/v) to about 40.0% (w/v), 0.001% (w/v) to about 30.0% (w/v), 0.001% (w/v) to about 20.0% (w/v), 0.001% (w/v) to about 10.0% (w/v) or about 0.01% (w/v) to about 90.0% (w/v), about 0.01% (w/v) to about 80.0% (w/v), about 0.01% (w/v) to about 70.0% (w/v), about 0.01% (w/v) to about 60.0% (w/v), about 0.01% (w/v) to about 50.0% (w/v), about 0.01% (w/v) to about 40.0% (w/v), about 0.01% (w/v) to about 30.0% (w/v)about 0.01% (w/v) to about 20.0% (w/v) or about 0.01% (w/v) to about 10.0% (w/v). In an embodiment, an inhaled formulations, a therapeutically effective amount of a therapeutic compound disclosed herein for an inhaled formulation may also be between 0.0001% (w/v) to about 90% (w/v), 0.0001% (w/v) to about 80% (w/v), 0.0001% (w/v) to about 70% (w/v), 0.0001% (w/v) to about 60% (w/v), 0.0001% (w/v) to about 50% (w/v), 0.0001% (w/v) to about 40% (w/v), 0.0001% (w/v) to about 30% (w/v), 0.0001% (w/v) to about 20% (w/v), 0.0001% (w/v) to about 10% (w/v), about 0.001% (w/v) to about 90.0% (w/v), 0.001% (w/v) to about 80.0% (w/v), 0.001% (w/v) to about 70.0% (w/v), 0.001% (w/v) to about 60.0% (w/v), 0.001% (w/v) to about 0.0% (w/v), 0.001% (w/v) to about 40.0% (w/v), 0.001% (w/v) to about 30.0% (w/v), 0.001% (w/v) to about 20.0% (w/v), 0.001% (w/v) to about 10.0% (w/v) or about 0.01% (w/v) to about 90.0% (w/v), about 0.01% (w/v) to about 80.0% (w/v), about 0.01% (w/v) to about 70.0% (w/v), about 0.01% (w/v) to about 60.0% (w/v), about 0.01% (w/v) to about 50.0% (w/v), about 0.01% (w/v) to about 40.0% (w/v), about 0.01% (w/v) to about 30.0% (w/v)about 0.01% (w/v) to about 20.0% (w/v) or about 0.01% (w/v) to about 10.0% (w/v).

In an embodiment, a therapeutic compound disclosed herein, or a composition comprising such a therapeutic compound, may be made into a solid formulation. In an embodiment, a solid formulations suitable for enteral or parenteral administration include, without limitation, capsules, tablets, pills, troches, lozenges, orally dissolving strips, powders and granules suitable for inhalation or for reconstitution into sterile injectable solutions or dispersions. In an embodiment, each of the aforementioned formulations can include, without limitation, an immediate release formulation, a slow release formulation (including without limitation, a wax matrix), beaded (including, without limitation, a double beaded wherein a bead releases immediately followed by another bead releasing at a later time), spheroidal oral drug absorption system (“SODAS”), an oral relase osmotic system (“OROS”), chewable tablet, a patch (including, without limitation, a delivery optimized thermodynamics (“DOT”)), sprinkles, or a prodrug. In an embodiment, a therapeutic compound or composition disclosed herein intended for such administration may be prepared according to any method known to the art for the manufacture of pharmaceutical compositions. In an embodiment, such solid dosage forms, the therapeutic compound may be admixed without limitation (a) at least one inert customary excipient (or carrier), such as without limitation, sodium citrate or dicalcium phosphate or (b) fillers or extenders, as for example, without limitation, starch, lactose, sucrose, glucose, mannitol, isomalt, and silicic acid, (c) binders, such as, without limitation, carboxymethylcellulose, alignates, gelatin, polyvinylpyrrolidone, sucrose and acacia, (d) humectants, such as, e.g., glycerol, (e) disintegrating agents, such as,without limitation, agar-agar, calcium carbonate, corn starch, potato starch, tapioca starch, alginic acid, certain complex silicates and sodium carbonate, (f) solution retarders, such as, without limitation, paraffin, (g) absorption accelerators, such as, without limitation, quaternary ammonium compounds, (h) wetting agents, such as, without limitation, cetyl alcohol and glycerol monostearate, (i) adsorbents, such as, without limitation, kaolin and bentonite, (j) lubricants, such as, without limitation, talc, stearic acid, calcium stearate, magnesium stearate, solid polyethylene glycols, sodium lauryl sulfate or mixtures thereof, and (k) buffering agents. In an embodiment, the tablets may be uncoated or they may be coated by known techniques to delay disintegration and absorption in the gastrointestinal tract and thereby provide a sustained action over a longer period. In an additional embodiment, without limitation, a time delay material such as glyceryl monostearate or glyceryl distearate may be employed. In an embodiment, in solid formulations, a therapeutically effective amount of a therapeutic compound disclosed herein typically may be between about 0.0001% (w/v) to about 90% (w/v), 0.0001% (w/v) to about 80% (w/v), 0.0001% (w/v) to about 70% (w/v), 0.0001% (w/v) to about 60% (w/v), 0.0001% (w/v) to about 50% (w/v), 0.0001% (w/v) to about 40% (w/v), 0.0001% (w/v) to about 30% (w/v), 0.0001% (w/v) to about 20% (w/v), 0.0001% (w/v) to about 10% (w/v), about 0.001% (w/v) to about 90.0% (w/v), 0.001% (w/v) to about 80.0% (w/v), 0.001% (w/v) to about 70.0% (w/v), 0.001% (w/v) to about 60.0% (w/v), 0.001% (w/v) to about 0.0% (w/v), 0.001% (w/v) to about 40.0% (w/v), 0.001% (w/v) to about 30.0% (w/v), 0.001% (w/v) to about 20.0% (w/v), 0.001% (w/v) to about 10.0% (w/v) or about 0.01% (w/v) to about 90.0% (w/v), about 0.01% (w/v) to about 80.0% (w/v), about 0.01% (w/v) to about 70.0% (w/v), about 0.01% (w/v) to about 60.0% (w/v), about 0.01% (w/v) to about 50.0% (w/v), about 0.01% (w/v) to about 40.0% (w/v), about 0.01% (w/v) to about 30.0% (w/v)about 0.01% (w/v) to about 20.0% (w/v) or about 0.01% (w/v) to about 10.0% (w/v).

In an embodiment, a therapeutic compound disclosed herein, or a composition comprising such a therapeutic compound, may be made into a semi-solid formulation. In an embodiment, a semi-solid formulations suitable for topical administration include, without limitation, ointments, creams, salves, and gels. In an embodiment, a therapeutic compound or composition disclosed herein intended for such administration may be prepared according to any method known to the art for the manufacture of pharmaceutical compositions. In an embodiment, in semi-solid formulations, a therapeutically effective amount of a therapeutic compound disclosed herein typically may be between about 0.0001% (w/v) to about 90% (w/v), 0.0001% (w/v) to about 80% (w/v), 0.0001% (w/v) to about 70% (w/v), 0.0001% (w/v) to about 60% (w/v), 0.0001% (w/v) to about 50% (w/v), 0.0001% (w/v) to about 40% (w/v), 0.0001% (w/v) to about 30% (w/v), 0.0001% (w/v) to about 20% (w/v), 0.0001% (w/v) to about 10% (w/v), about 0.001% (w/v) to about 90.0% (w/v), 0.001% (w/v) to about 80.0% (w/v), 0.001% (w/v) to about 70.0% (w/v), 0.001% (w/v) to about 60.0% (w/v), 0.001% (w/v) to about 0.0% (w/v), 0.001% (w/v) to about 40.0% (w/v), 0.001% (w/v) to about 30.0% (w/v), 0.001% (w/v) to about 20.0% (w/v), 0.001% (w/v) to about 10.0% (w/v) or about 0.01% (w/v) to about 90.0% (w/v), about 0.01% (w/v) to about 80.0% (w/v), about 0.01% (w/v) to about 70.0% (w/v), about 0.01% (w/v) to about 60.0% (w/v), about 0.01% (w/v) to about 50.0% (w/v), about 0.01% (w/v) to about 40.0% (w/v), about 0.01% (w/v) to about 30.0% (w/v)about 0.01% (w/v) to about 20.0% (w/v) or about 0.01% (w/v) to about 10.0% (w/v). In an embodiment, in semi-solid formulations, a therapeutically effective amount of a therapeutic compound disclosed herein typically may also be between about 0.0001% (w/v) to about 90% (w/v), 0.0001% (w/v) to about 80% (w/v), 0.0001% (w/v) to about 70% (w/v), 0.0001% (w/v) to about 60% (w/v), 0.0001% (w/v) to about 50% (w/v), 0.0001% (w/v) to about 40% (w/v), 0.0001% (w/v) to about 30% (w/v), 0.0001% (w/v) to about 20% (w/v), 0.0001% (w/v) to about 10% (w/v), about 0.001% (w/v) to about 90.0% (w/v), 0.001% (w/v) to about 80.0% (w/v), 0.001% (w/v) to about 70.0% (w/v), 0.001% (w/v) to about 60.0% (w/v), 0.001% (w/v) to about 0.0% (w/v), 0.001% (w/v) to about 40.0% (w/v), 0.001% (w/v) to about 30.0% (w/v), 0.001% (w/v) to about 20.0% (w/v), 0.001% (w/v) to about 10.0% (w/v) or about 0.01% (w/v) to about 90.0% (w/v), about 0.01% (w/v) to about 80.0% (w/v), about 0.01% (w/v) to about 70.0% (w/v), about 0.01% (w/v) to about 60.0% (w/v), about 0.01% (w/v) to about 50.0% (w/v), about 0.01% (w/v) to about 40.0% (w/v), about 0.01% (w/v) to about 30.0% (w/v)about 0.01% (w/v) to about 20.0% (w/v) or about 0.01% (w/v) to about 10.0% (w/v).

In an embodiment, a therapeutic compound disclosed herein, or a composition comprising such a therapeutic compound, may be made into a liquid formulation. In an embodiment, liquid formulations suitable for enteral or parenteral administration include, without limitation, solutions, syrups, elixirs, dispersions, emulsions, and suspensions. In an embodiment, a therapeutic compound or composition disclosed herein intended for such administration may be prepared, without limitation, according to any method known to the art for the manufacture of pharmaceutical compositions. In an embodiment, in such liquid dosage forms, a therapeutic compound or composition disclosed herein may be admixed with, without limitation, (a) suitable aqueous and nonaqueous carriers, (b) diluents, (c) solvents, such as, without limitation, water, ethanol, propylene glycol, polyethyleneglycol, glycerol, vegetable oils, such as, without limitation, rapeseed oil and olive oil, and injectable organic esters such as ethyl oleate; and/or fluidity agents, such as, without limitation, surfactants or coating agents like lecithin. In the case of dispersions and suspensions, fluidity can also be controlled by maintaining a particular particle size. In an embodiment, in liquid formulations, a therapeutically effective amount of a therapeutic compound disclosed herein typically may be between about 0.0001% (w/v) to about 90% (w/v), 0.0001% (w/v) to about 80% (w/v), 0.0001% (w/v) to about 70% (w/v), 0.0001% (w/v) to about 60% (w/v), 0.0001% (w/v) to about 50% (w/v), 0.0001% (w/v) to about 40% (w/v), 0.0001% (w/v) to about 30% (w/v), 0.0001% (w/v) to about 20% (w/v), 0.0001% (w/v) to about 10% (w/v), about 0.001% (w/v) to about 90.0% (w/v), 0.001% (w/v) to about 80.0% (w/v), 0.001% (w/v) to about 70.0% (w/v), 0.001% (w/v) to about 60.0% (w/v), 0.001% (w/v) to about 0.0% (w/v), 0.001% (w/v) to about 40.0% (w/v), 0.001% (w/v) to about 30.0% (w/v), 0.001% (w/v) to about 20.0% (w/v), 0.001% (w/v) to about 10.0% (w/v) or about 0.01% (w/v) to about 90.0% (w/v), about 0.01% (w/v) to about 80.0% (w/v), about 0.01% (w/v) to about 70.0% (w/v), about 0.01% (w/v) to about 60.0% (w/v), about 0.01% (w/v) to about 50.0% (w/v), about 0.01% (w/v) to about 40.0% (w/v), about 0.01% (w/v) to about 30.0% (w/v)about 0.01% (w/v) to about 20.0% (w/v) or about 0.01% (w/v) to about 10.0% (w/v).

In an embodiment, syrups and elixirs may be formulated, without limitation, sweetening agents, for example glycerol, propylene glycol, sorbitol or sucrose. In an additional embodiment, such formulations may also contain, without limitation, a demulcent, a preservative, flavoring agents, and coloring agents.

In an embodiment, liquid suspensions may be formulated, without limitation, by suspending a therapeutic compound disclosed herein in admixture with excipients suitable for the manufacture of aqueous suspensions. In an embodiment, such excipients are suspending agents, for example, without limitation, sodium carboxymethylcellulose, methylcellulose, hydroxypropylmethylcellulose, sodium alginate, pectin, polyvinyl pyrrolidone, polyvinyl alcohol, natural gum, agar, gum tragacanth and gum acacia; dispersing or wetting agents may be a naturally occurring phosphatide, for example lecithin, or condensation products of an alkylene oxide with fatty acids, for example, without limitation, polyoxyethylene stearate, or condensation products of ethylene oxide with long-chain aliphatic alcohols, for example, without limitation, heptadecaethyleneoxycetanol, or condensation products of ethylene oxide with partial esters derived from fatty acids, for example, without limitation, polyoxyethylene sorbitan monooleate.

In an embodiment, oily suspensions may be formulated by suspending a therapeutic compound disclosed herein in admixture with (a) vegetable oils, such as, without limitation, almond oil, arachis oil, avocado oil, canola oil, castor oil, coconut oil, corn oil, cottonseed oil, grape seed oil, hazelnut oil, hemp oil, linseed oil, olive oil, palm oil, peanut oil, rapeseed oil, rice bran oil, safflower oil, sesame oil, soybean oil, soya oil, sunflower oil, walnut oil, wheat germ oil, or a combination thereof, (b) a saturated fatty acid, an unsaturated fatty acid, or a combination thereof, such as, without limitation, palmitic acid, stearic acid, oleic acid, linoleic acid, linolenic acid, or a combination thereof, (c) mineral oil such as, without limitation, liquid paraffin, (d) surfactants or detergents. In an embodiment, the oily suspensions may contain a thickening agent, for example beeswax, hard paraffin or cetyl alcohol. In an embodiment, sweetening agents, such as those set forth above, and flavoring agents may be added to provide a palatable oral preparation. In an embodiment, these compositions may be preserved by the addition of an antioxidant such as ascorbic acid.

In an embodiment, dispersible powders and granules suitable for preparation of an aqueous suspension by the addition of water provide the combined therapeutic compounds in admixture with a dispersing or wetting agent, suspending agent and one or more preservatives.

In an embodiment, a therapeutic compound disclosed herein may be in the form of oil-in-water emulsions. In an embodiment, the oily phase may be a vegetable oil as disclosed herein or a mineral oil as disclosed herein or mixtures thereof. In an additional embodiment, suitable emulsifying agents may be naturally occurring gums, such as, without limitation, gum acacia or gum tragacanth, naturally occurring phosphatides, for example soya bean, lecithin, and esters or partial esters derived from fatty acids and hexitol anhydrides, for example, without limitation, sorbitan monooleate and condensation products of the said partial esters with ethylene oxide, for example polyoxyethylene sorbitan monooleate.

In an embodiment, a therapeutic compound disclosed herein, or a composition comprising such a therapeutic compound, may also be incorporated into a drug delivery platform in order to achieve a controlled release profile over time. In an embodiment, such a drug delivery platform comprises a therapeutic compound disclosed herein dispersed within a polymer matrix, typically, without limitation, a biodegradable, bioerodible, and/or bioresorbable polymer matrix. In an embodiment, as used herein, the term “polymer” refers to synthetic homo- or copolymers, naturally occurring homo- or copolymers, as well as, without limitation, synthetic modifications or derivatives thereof having a linear, branched or star structure. In an embodiment, copolymers can be arranged in any form, such as, without limitation, random, block, segmented, tapered blocks, graft, or triblock. In an embodiment, polymers are generally condensation polymers. In an embodiment, polymers can be further modified to enhance their mechanical or degradation properties by introducing cross-linking agents or changing the hydrophobicity of the side residues. In an embodiment, if crosslinked, polymers are usually less than 75% crosslinked, 65% crosslinked, 55% crosslinked, 45% crosslinked, 35% crosslinked, 25% crosslinked, 15% crosslinked 5% crosslinked, usually less than 1% crosslinked.

In an embodiment, suitable polymers include, without limitation, alginates, aliphatic polyesters, polyalkylene oxalates, polyamides, polyamidoesters, polyanhydrides, polycarbonates, polyesters, polyethylene glycol, polyhydroxyaliphatic carboxylic acids, polyorthoesters, polyoxaesters, polypeptides, polyphosphazenes, polysaccharides, and polyurethanes. In an embodiment, the polymer usually comprises at least about 10% (w/w), at least about 20% (w/w), at least about 30% (w/w), at least about 40% (w/w), at least about 50% (w/w), at least about 60% (w/w), at least about 70% (w/w), at least about 80% (w/w), or at least about 90% (w/w) of the drug delivery platform. In an embodiment, examples of biodegradable, bioerodible, and/or bioresorbable polymers and methods useful to make a drug delivery platform are described in, e.g., Drost, without limitation, Controlled Release Formulation, U.S. Pat. No. 4,756,911; Smith, et. al., Sustained Release Drug Delivery Devices, U.S. Pat. No. 5,378,475; Wong and Kochinke, Formulation for Controlled Release of Drugs by Combining Hyrophilic and Hydrophobic Agents, U.S. Pat. No. 7,048,946; Hughes, et. al., Compositions and Methods for Localized Therapy of the Eye, U.S. Patent Publication 2005/0181017; Hughes, Hypotensive Lipid-Containing Biodegradable Intraocular Implants and Related Methods, U.S. Patent Publication 2005/024444; Altman, et al., Silk Fibroin Hydrogels and Uses Thereof, U.S. Patent Publication 2011/0008437; each of which is incorporated by reference in its entirety.

In an embodiment, a polymer composing the matrix is a polypeptide such as, without limitation, silk fibroin, keratin, or collagen. In an additional embodiment, a polymer composing the matrix is a polysaccharide such as, without limitation, cellulose, agarose, elastin, chitosan, chitin, or a glycosaminoglycan like chondroitin sulfate, dermatan sulfate, keratan sulfate, or hyaluronic acid. In yet another embodiment, a polymer composing the matrix is a polyester such as, without limitation, D-lactic acid, L-lactic acid, racemic lactic acid, glycolic acid, caprolactone, and combinations thereof.

One of ordinary skill in the art appreciates that the selection of a suitable polymer for forming a suitable disclosed drug delivery platform depends on several factors. The more relevant factors in the selection of the appropriate polymer(s), include, without limitation, compatibility of polymer with drug, desired release kinetics of drug, desired biodegradation kinetics of platform at implantation site, desired bioerodible kinetics of platform at implantation site, desired bioresorbable kinetics of platform at implantation site, in vivo mechanical performance of platform, processing temperatures, biocompatibility of platform, and patient tolerance. Other relevant factors that, to some extent, without limitation, dictate the in vitro and in vivo behavior of the polymer include the chemical composition, spatial distribution of the constituents, the molecular weight of the polymer and the degree of crystallinity

### Administration

In one embodiment a conjugated compound of the present invention may be administered by injection, such as intravenous, intramuscular, or intraperitoneal injection. The compositions may be useful as therapeutic, diagnostic and/or similar agents

To administer compositions comprising a conjugated compound of the present invention to human or test animals, in one aspect, the compositions comprise one or more pharmaceutically acceptable carriers. The terms “pharmaceutically” or “pharmacologically acceptable” refer to molecular entities and compositions that are stable, inhibit protein degradation such as aggregation and cleavage products, and in addition do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like, including those agents disclosed above.

As used herein, “effective amount” includes a dose suitable for treating a mammal having a clinically defined disorder.

The compositions may be administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramuscular, intramammary, intraperitoneal, intrathecal, retrobulbar, intrapulmonary injection and or surgical implantation at a particular site is contemplated as well. Generally, compositions are essentially free of pyrogens, as well as other impurities that could be harmful to the recipient.

Single or multiple administrations of the compositions can be carried out with the dose levels and pattern being selected by the treating physician. For the prevention or treatment of disease, the appropriate dosage will depend on the type of disease to be treated, as described above, the severity and course of the disease, whether drug is administered for preventive or therapeutic purposes, previous therapy, the patient's clinical history and response to the drug, and the discretion of the attending physician.

The present invention also relates to a pharmaceutical composition comprising an effective amount of a conjugated compound or protein as defined herein. The pharmaceutical composition may further comprise a pharmaceutically acceptable carrier, diluent, salt, buffer, or excipient. The pharmaceutical composition can be used for treating clinically-defined disorders. The pharmaceutical composition of the invention may be a solution or a lyophilized product. Solutions of the pharmaceutical composition may be subjected to any suitable lyophilization process.

As an additional aspect, the invention includes kits which comprise a composition of the invention packaged in a manner which facilitates its use for administration to subjects. In one embodiment, such a kit includes a compound or composition described herein (e.g., a composition comprising a conjugated protein), packaged in a container such as a sealed bottle or vessel, with a label affixed to the container or included in the package that describes use of the compound or composition in practicing the method. In one embodiment, the kit contains a first container having a composition comprising a conjugated protein and a second container having a physiologically acceptable reconstitution solution for the composition in the first container. In one aspect, the compound or composition is packaged in a unit dosage form. The kit may further include a device suitable for administering the composition according to a specific route of administration. Preferably, the kit contains a label that describes use of the therapeutic protein or peptide composition.

In one embodiment, the derivative retains the full functional activity of native therapeutic compounds, and provides an extended half-life in vivo, as compared to native therapeutic compounds. In another embodiment, the derivative retains at least 20, 21, 22, 23, 24, 25, 26, 27, 28, 29, 30, 31, 32, 34, 35, 36, 37, 38, 39, 40, 41, 42, 43, 44. 45, 46, 47, 48, 49, 50, 51, 52, 53, 54, 55, 56,57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 200, or 500 percent (%) biological activity relative to native compound.

In an embodiment, a drug delivery platform includes both a sustained release drug delivery platform and an extended release drug delivery platform. In an embodiment, the term “sustained release” refers to the release of a therapeutic compound or compounds disclosed herein over a period of about seven days or more. In an embodiment, the term “extended release” refers to the release of a therapeutic compound or compounds disclosed herein over a period of time of less than about seven days.

In an embodiment, a sustained release drug delivery platform releases a therapeutic compound over a period of, without limitation, about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In another embodiment, a sustained release drug delivery platform releases a therapeutic compound disclosed herein with substantially zero order release kinetics over a period , without limitation, at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

In an embodiment, a PSA-therapeutic compound is in the form of a long acting composition that includes, without limitation, extended release compositions. An embodiment includes, without limitation, an extended release capsule, tablet or other solid or a liquid formulation that provides the therapeutic compound or compounds to the patient to whom it is administered over time. The long acting composition can provide in a patient for 4 hours, 6 hours, 8 hours, 10 hours, 12 hours, 16 hours, 20 hours, 24 hours, 28 hours, 30 hours, 32 hours, 34 hours, 36 hours, 40 hours, 48 hours, 3 days, 4 days, 5 days, 6 days, 7 days, 2 weeks, 3 weeks or 4 weeks. The long acting formulations can provide activity of a polysiaylated therapeutic compound for as little as 4 hours or as long as 4 weeks, for as little as 4 hours or as long as 3 weeks, for as little as 4 hours or as long as 2 weeks, for as little as 4 hours or as long as 1 week, for as little as 4 hours or as long as 6 days, for as little as 4 hours or as long as 5 days, for as little as 4 hours or as long as 4 days, for as little as 4 hours or as long as 3 days, for as little as 4 hours or as long as 2 days, for as little as 4 hours or as long as 1 day, for as little as 4 hours or as long as 20 hours, for as little as 4 hours or as long as 16 hours, for as little as 4 hours or as long as 14 hours, for as little as 4 hours or as long as 12 hours, for as little as 4 hours or as long as 10 hours, for as little as 4 hours or as long as 8 hours, for as little as 4 hours or as long as 6 hours.

In an embodiment, a sustained release drug delivery platform releases a therapeutic compound or compounds disclosed herein over a period of, without limitation, about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In other aspects of this embodiment, a sustained release drug delivery platform releases a therapeutic compound or compounds disclosed herein with substantially first order release kinetics over a period of, without limitation, at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

In an embodiment, a drug delivery platform releases a therapeutic compound or compounds disclosed herein over a period of, without limitation, about 1 day after administration, about 2 days after administration, about 3 days after administration, about 4 days after administration, about 5 days after administration, about 6 days after administration or about 7 days or more after administration. In an additional embodiment, a drug delivery platform releases a therapeutic compound or compounds disclosed herein with substantially zero order release kinetics over a period of, without limitation, at most 1 day after administration, at most 2 days after administration, at most 3 days after administration, at most 4 days after administration, at most 5 days after administration, at most 6 days after administration or at most 7 days or more after administration.

In an embodiment, a drug delivery platform releases a therapeutic compound or compounds disclosed herein with release kinetics over a period of, without limitation, about 1 day after administration, about 2 days after administration, about 3 days after administration, about 4 days after administration, about 5 days after administration, about 6 days after administration or about 7 days or more after administration. In an additional embodiment, a drug delivery platform releases a therapeutic compound or compounds disclosed herein with substantially first order release kinetics over a period of, e.g., at most 1 day after administration, at most 2 days after administration, at most 3 days after administration, at most 4 days after administration, at most 5 days after administration, at most 6 days after administration or at most 7 days or more after administration.

### Sialic Acid and PSA

As used herein, “sialic acid moieties” includes sialic acid monomers or polymers (“polysaccharides”) which are soluble in an aqueous solution or suspension and have little or no negative impact, such as side effects, to mammals upon administration of the PSA-protein conjugate in a pharmaceutically effective amount. PSA and mPSA are characterized, in one aspect, as having 1, 2, 3, 4, 5, 10, 20, 30, 40, 50, 60, 70, 80, 90, 100, 125, 150, 200, 300, 400, or 500 sialic acid units. In certain aspects, different sialic acid units are combined in a chain.

In one embodiment of the invention, the sialic acid portion of the PSA or mPSA compound is highly hydrophilic, and in another embodiment the entire compound is highly hydrophilic. Hydrophilicity is conferred primarily by the pendant carboxyl groups of the sialic acid units, as well as the hydroxyl groups. The saccharide unit may contain other functional groups, such as, amine, hydroxyl or sulphate groups, or combinations thereof. These groups may be present on naturally-occurring saccharide compounds, or introduced into derivative polysaccharide compounds. The PSA and mPSA used in the methods and conjugates of the invention may be further characterized as described above in the Background of the Invention.

The naturally occurring polymer PSA is available as a polydisperse preparation showing a broad size distribution (e.g. Sigma C-5762) and high polydispersity (PD). Because the polysaccharides are usually produced in bacteria carrying the inherent risk of copurifying endotoxins, the purification of long sialic acid polymer chains may raise the probability of increased endotoxin content. Short PSA molecules with 1-4 sialic acid units can also be synthetically prepared (Kang S H et al., Chem Commun. 2000;227-8; Ress D K and Linhardt R J, Current Organic Synthesis. 2004;1:31-46), thus minimizing the risk of high endotoxin levels. However, PSA preparations with a narrow size distribution and low polydispersity, which are also endotoxin-free, can now be manufactured. Polysaccharide compounds of particular use for the invention are, in one aspect, those produced by bacteria. Some of these naturally-occurring polysaccharides are known as glycolipids. In one embodiment, the polysaccharide compounds are substantially free of terminal galactose units.

In various embodiments, the compound is linked to or associated with the PSA or mPSA compound in stoichiometric amounts (e.g., 1:1, 1:2, 1:3, 1:4, 1:5, 1:6, 1:7, 1:7, 1:8, 1:9, or 1:10, etc.). In various embodiments, 1-6, 7-12 or 13-20 PSA and/or mPSA units are linked to the compound. In still other embodiments, 1, 2, 3, 4, 5, 6, 7, 8, 9, 10, 11, 12, 13, 14, 15, 16, 17, 18, 19, 20 or more PSA and/or mPSA units are linked to the compound.

Optionally, the compound is modified to introduce glycosylation sites (i.e., sites other than the native glycosylation sites). Such modification may be accomplished using standard molecular biological techniques known in the art. Moreover, the compound, prior to conjugation via one or more carbohydrate moieties, may be glycosylated in vivo or in vitro.

### Aminooxy Linkage

In one embodiment of the invention, the reaction of hydroxylamine or hydroxylamine derivatives with aldehydes (e.g., on a carbohydrate moiety following oxidation by sodium periodate) to form an oxime group is applied to the preparation of conjugates of compound. For example, a glycoprotein is first oxidized with a oxidizing agent such as sodium periodate (NaIO₄) (Rothfus J A et Smith E L., J Biol Chem 1963, 238, 1402-10; and Van Lenten L and Ashwell G., J Biol Chem 1971, 246, 1889-94). The periodate oxidation of e.g. glycoproteins is based on the classical Malaprade reaction described in 1928, the oxidation of vicinal diols with periodate to form an active aldehyde group (Malaprade L., Analytical application, Bull Soc Chim France, 1928, 43, 683-96). Additional examples for such an oxidizing agent are lead tetraacetate (Pb(OAc)₄), manganese acetate (MnO(Ac)₃), cobalt acetate (Co(OAc)₂), thallium acetate (T10Ac), cerium sulfate (Ce(SO₄)₂) (U.S. Pat. No. 4,367,309) or potassium perruthenate (KRuO₄) (Marko et al., J Am Chem Soc 1997,119, 12661-2). By “oxidizing agent” a mild oxidizing compound which is capable of oxidizing vicinal diols in carbohydrates, thereby generating active aldehyde groups under physiological reaction conditions is meant.

The second step is the coupling of the polymer containing an aminooxy group to the oxidized carbohydrate moiety to form an oxime linkage In one embodiment of the invention, this step can be carried out in the presence of catalytic amounts of the nucleophilic catalyst aniline or aniline derivatives (Dirksen A., Dawson P E, Bioconjugate Chem. 2008; Zeng Y et al., Nature Methods 2009;6:207-9). The aniline catalysis dramatically accelerates the oxime ligation allowing the use of very low concentrations of the reagents. In another embodiment of the invention the oxime linkage is stabilized by reduction with NaCNBH₃ to form an alkoxyamine linkage.

In one embodiment of the invention, the reaction steps to conjugate PSA or mPSA to a protein are carried out separately and sequentially (i.e., starting materials (e.g., protein, polymer, etc), reagents (e.g., oxidizing agents, aniline, etc) and reaction products (e.g., oxidized carbohydrate on a protein, activated aminooxy polymer, etc) are separated between individual reaction steps).

Additional information on aminooxy technology can be found in the following references, each of which is incorporated in their entireties: EP 1681303A1 (HASylated erythropoietin); WO 2005/014024 (conjugates of a polymer and a protein linked by an oxime linking group); WO96/40662 (aminooxy-containing linker compounds and their application in conjugates); WO 2008/025856 (Modified proteins); Peri F et al., Tetrahedron 1998, 54, 12269-78; Kubler-Kielb J and Pozsgay V., J Org Chem 2005, 70, 6887-90; Lees A et al., Vaccine 2006, 24(6), 716-29; and Heredia K L et al., Macromoecules 2007, 40(14), 4772-9.

Additional information on attachment of a PSA to antibodies can be found in the following references, each of which is incorporated in their entireties:; Konterman R. and Dubel S., Antibody Engineering, 2010, V.1, 2^{nd} ed. Springer Protocals, ISBN-13:978-3642011436; Zhang B, et al.: Unveiling a glycation hot spot in a recombinant humanized monoclonal antibody, Anal Chem., 2008, 80, 2379-2390; Miller A K, et al. Characterization of site-specific glycation during process development of a human therapeutic monoclonal antibody, J. Pharm. Sci., 2011, 100, 2543-2550.

Advantages of the invention include high recovery of conjugate, high retention of activity of the conjugated glycoprotein compared to unconjugated protein and high conjugation efficiency.

### Bioactivity Assays

A variety of bioassays are useful for determining the effect of polysialylation on activity of the target protein. Comparisons are made between the target protein prior to modification, and after attachment of polysialyic acid/PSA. For example, enzymes which are polysialylated herein are particularly suited to testing activity of the modified target protein by testing enzyme activity. For antibodies or binding fragment thereof which are polysialylated herein, standard antibody-ligand binding assays are utilized and modified and unmodified antibodies are compared. For other target proteins, a bioassay for the target may be utilized to measure activity of a polysialylated target protein herein. An appropriate and sensitive assay is required for the particular target protein. The assay should also provide a specific readout for the polysialylated target protein without interference by related compounds. Bioassay methods applicable to a wide variety of targets are described in Examples herein. Particular bioassays relating to a particular target protein that is polysialylated according to the invention are also described herein above in the patents and applications mentioned and incorporated by reference herein as related to particular target proteins.

Therapeutic-PSA proteins herein can be identified using various in vitro assays. Preferably, the assays are high-throughput assays that allow for screening multiple candidates simultaneously. In some embodiments, biomolecular interactions can be monitored in real time with the BIACORE® system, which uses SPR to detect changes in the resonance angle of light at the surface of a thin gold film on a glass support due to changes in the refractive index of the surface up to 300 nm away. BIACORE® analysis generates association rate constants, dissociation rate constants, equilibrium dissociation constants, and affinity constants. Binding affinity is obtained by assessing the association and dissociation rate constants using a BIACORE® surface plasmon resonance system (Biacore, Inc.). A biosensor chip is activated for covalent coupling of the target. The target is then diluted and injected over the chip to obtain a signal in response units of immobilized material. Since the signal in resonance units (RU) is proportional to the mass of immobilized material, this represents a range of immobilized target densities on the matrix.

Cell-based assays can be used to characterize, measure and compare the biological activity of various polysyliated proteins provided herein. In some embodiments, the cell-based assay is an enzyme-linked immunosorbent assay (ELISA). ELISA kits are commercially available from numerous sources, such as Cell Sciences.RTM. (Canton, Mass.). Methods for using ELISA kits are known in the art and the kits generally include instruction manuals, e.g., on how to prepare samples, standards, calibration curves, and conduct experiments. In another embodiment, the cell-based assay is a homogeneous time-resolved fluorescence assay (HTRF.RTM.). HTRF.RTM. kits are commercially available from Cisbio International (Bedford, Mass.). Methods for using HTRF.RTM. kits are known in the art and the kits generally include instruction manuals, e.g., on how to prepare samples, standards, calibration curves, and conduct experiments. Homogeneous time-resolved fluorescence cell-based assays are described in U.S. Pat. No. 5,527,684, the disclosure of which is incorporated by reference herein, and Document Reference No. 62AM4PEB rev02 (August 2007) available from Cisbio HTRF.RTM.Product Center. See www.htrf.com/products/gper/camp/, the disclosure of which is incorporated by reference herein.

The invention is now illustrated with reference to the following examples.

### EXAMPLES

### Example 1

### Preparation of Aminooxy-PSA Polymer

1.3 g of oxidized colominic acid (23 kDa) was dissolved in 18 ml of 50 mM sodium acetate pH 5.5±0.02. 20 fold molar excess of 1, 11-diamino-3,6,9-trioxaundecane (also referred to as 3,6,9-trioxa-undecane-1,11-dioxyamine) was dissolved in minimum amount of 50 mM sodium acetate (pH 5.5±0.02) and was added to the PSA solution. The final colominic acid concentration was 62.5 mg/ml. This reaction mixture was incubated for 2±0.1 hr at 22±1.0° C. on a gentle mixer (22 oscillations per minute). After this, 0.65 ml of 160 mg/ml NaCNBH₃ solution was added to the above reaction mixture so as to make the final concentration of 5.00 mg/ml. This was incubated for 3.0±0.20 hours at 4.0±1.0° C. on a shaker (22 oscillations per minute) in a endotoxin free air tight container with enough headspace for mixing. For the purification, the sample was diluted with 2 mM triethanolamine, pH 8.0±0.02 to make final colominic acid concentration of 20 mg/ml. The reaction mixture was desalted to remove excess of 1,11-diamino-3,6,9-trioxaundecane, NaCNBH₃ and byproducts of the reaction. This was followed by desalting on a Sephadex G25 column using 20 mM triethanolamine buffer (pH 8.0±0.02). The pH of the desalted sample was adjusted to pH 7.8-8.0 and was ultrafiltered/diafiltered with 20 mM TEA pH 8.0 once and 2 mM triethanolamine (TEA) pH 8.0 twice. The sample was freeze dried and stored at −80° C.

Alternatively, purification was done in presence of high salt during desalting and ultrafiltration/diafiltration (UF/DF) steps. Anion exchange chromatography in high salt was also used to make highly pure aminooxy-PSA. By analogy, different molecular weights of aminooxy-PSA were synthesized.

### Example 2

### Coupling of Diaminooxy-PSA to Erythropoietin (EPO)

0.2 mg of EPO was oxidized with 10 mM of NaIO₄ for 30 minutes at 4° C. The oxidation was stopped by adding NaHSO₃ to a final concentration of 5 mM. The conjugation reaction was carried out using the oxidized EPO with diaminooxy polymer of 23 kDa. The final concentration of polymer in the reaction mixture was 1.25 mM. The final concentration of EPO in the reaction mixture was 0.125 mg/ml. The final pH of the reaction mixture was around 5.75. Sodium cyanoborohydride was added to the reaction mixture to a concentration of 50 mM or 3.17 mg/ml. The reaction was carried out at 4° C. for 24 hours. Unpurified conjugate was characterized using SDS PAGE. A shift in the band was seen for the conjugate in SDS PAGE.

### Example 3

### Coupling of Diaminooxy-PSA to EPO with Aniline to Act as a Nucleophilic Catalyst

0.2 mg of EPO was oxidized with 10 mM NaIO₄ for 30 minutes at 4° C. The oxidation was stopped by adding NaHSO₃ to a final concentration of 5 mM. The conjugation reaction was carried out using the oxidized EPO with diaminooxy PSA polymer (22 kDa). The final concentration of polymer in the reaction mixture was 1.25 mM. The final pH of the reaction mixture was around 5.75. Sodium cyanoborohydride was added to the reaction mixture to a concentration of 50 mM or 3.17 mg/ml. The final protein concentration in the reaction was 0.125 mg/ml. 84.21 μl of 200 mM aniline solution was added to the 1.6 ml of reaction mixture. The reaction was carried out at 4° C. for overnight. The conjugates were characterized using SDS PAGE. A shift in the band was seen in the conjugates. No adverse effect of aniline was observed on activity of the conjugates.

### Example 4

### Coupling of Hydrazide-PSA to Erythropoietin

For oxidation of erythropoietin (EPO), NaIO₄ was used at a concentration of 10 mM. EPO (1 mg) was oxidized at pH 5.75 at 4° C. for 30 minutes then oxidation was stopped by adding NaHSO₃ to a final concentration of 5 mM. The conjugation reaction was carried out using the oxidized EPO with hydrazide-PSA polymer. The molecular weight of the hydrazide-PSA used for conjugation was 24.34 kDa. The final concentration of hydrazide-PSA in the reaction mixture was 1.25 mM. The final concentration of EPO in the reaction mixture was 0.125 mg/ml. The final pH of the reaction mixture was around 5.75. Sodium cyanoborohydride was added to the reaction mixture to a concentration of 50 mM or 3.17 mg/ml. The reaction was carried out at 4° C. for 24 hours. Conjugates were characterized using SDS PAGE and western blotting. A shift in the band was seen for the conjugate in SDS PAGE and a positive result was obtained from western blotting.

### Example 5

### Coupling of Aminooxy-PSA to NGF

To 10 mg nerve growth factor (NGF), dissolved in 10 ml Hepes buffer pH 6 (50 mM Hepes, 5 mM CaCl₂, 150 mM NaCl, 0.01% Tween) 50 μl 10 mM sodium periodate was added. The mixture was shaken in the dark for 30 min at 4° C. and quenched for 30 min at 4° C. by the addition of 100 μl of an aqueous1M glycerol solution. Then 20.0 mg aminooxy-PSA (18.8 kD) was added and the mixture was shaken over night at 4° C. The ionic strength was increased by adding a buffer containing 8M ammonium acetate (8M ammonium acetate, 50 mM Hepes, 5 mM CaCl₂, 350 mM NaCl, 0.01% Tween 80, pH 6.9) to get a final concentration of 2.5M ammonium acetate. Next, the reaction mixture was loaded on a HiTrap Butyl FF (GE Healthcare, Fairfield, Conn.) column which was equilibrated with equilibration buffer (2.5M ammonium acetate, 50 mM Hepes, 5 mM CaCl₂, 350 mM NaCl, 0.01% Tween 80, pH 6.9). The product was eluted with elution buffer (50 mM Hepes, 5 mM CaCl₂, 0.01% Tween 80, pH 7.4), and the eluate was concentrated by centrifugal filtration using Vivaspin (Sartorius, Goettingen, Germany) devices with 30,000 MWCO.

### Example 6

### Coupling of Aminooxy-PSA to Interferon-Alpha (IFN-Alpha)

To 10 mg interferon-alpha (IFN-alpha), dissolved in 10 ml Hepes buffer pH 6 (50 mM Hepes, 5 mM CaCl₂, 150 mM NaCl, 0.01% Tween) 50 μl 10 mM sodium periodate was added. The mixture was shaken in the dark for 30 min at 4° C. and quenched for 30 min at 4° C. by the addition of 100 μl of an aqueous1M glycerol solution. Then 20.0 mg aminooxy-PSA (18.8 kD) was added and the mixture was shaken over night at 4° C. The ionic strength was increased by adding a buffer containing 8M ammonium acetate (8M ammonium acetate, 50 mM Hepes, 5 mM CaCl₂, 350 mM NaCl, 0.01% Tween 80, pH 6.9) to get a final concentration of 2.5M ammonium acetate. Next, the reaction mixture was loaded on a HiTrap Butyl FF (GE Healthcare, Fairfield, Conn.) column which was equilibrated with equilibration buffer (2.5M ammonium acetate, 50 mM Hepes, 5 mM CaCl₂, 350 mM NaCl, 0.01% Tween 80, pH 6.9). The product was eluted with elution buffer (50 mM Hepes, 5 mM CaCl₂, 0.01% Tween 80, pH 7.4), and the eluate was concentrated by centrifugal filtration using Vivaspin (Sartorius, Goettingen, Germany) devices with 30,000 MWCO.

### Example 7

### Coupling of Aminooxy-PSA to TPA (Tissue Plasminogen Activator)

To 10 mg TPA, dissolved in 10 ml Hepes buffer pH 6 (50 mM Hepes, 5 mM CaCl₂, 150 mM NaCl, 0.01% Tween) 50 μl 10 mM sodium periodate was added. The mixture was shaken in the dark for 30 min at 4° C. and quenched for 30 min at 4° C. by the addition of 100 μl of an aqueous1M glycerol solution. Then 20.0 mg aminooxy-PSA (18.8 kD) was added and the mixture was shaken over night at 4° C. The ionic strength was increased by adding a buffer containing 8M ammonium acetate (8M ammonium acetate, 50 mM Hepes, 5 mM CaCl₂, 350 mM NaCl, 0.01% Tween 80, pH 6.9) to get a final concentration of 2.5M ammonium acetate. Next, the reaction mixture was loaded on a HiTrap Butyl FF (GE Healthcare, Fairfield, Conn.) column which was equilibrated with equilibration buffer (2.5M ammonium acetate, 50 mM Hepes, 5 mM CaCl₂, 350 mM NaCl, 0.01% Tween 80, pH 6.9). The product was eluted with elution buffer (50 mM Hepes, 5 mM CaCl₂, 0.01% Tween 80, pH 7.4), and the eluate was concentrated by centrifugal filtration using Vivaspin (Sartorius, Goettingen, Germany) devices with 30,000 MWCO.

### Example 8

### Coupling of Aminooxy-PSA to Thrombopoietin (TPO)

To 10 mg TPA, dissolved in 10 ml Hepes buffer pH 6 (50 mM Hepes, 5 mM CaCl₂, 150 mM NaCl, 0.01% Tween) 50 μl 10 mM sodium periodate was added. The mixture was shaken in the dark for 30 min at 4° C. and quenched for 30 min at 4° C. by the addition of 100 μl of an aqueous1M glycerol solution. Then 20.0 mg aminooxy-PSA (18.8 kD) was added and the mixture was shaken over night at 4° C. The ionic strength was increased by adding a buffer containing 8M ammonium acetate (8M ammonium acetate, 50 mM Hepes, 5 mM CaCl₂, 350 mM NaCl, 0.01% Tween 80, pH 6.9) to get a final concentration of 2.5M ammonium acetate. Next, the reaction mixture was loaded on a HiTrap Butyl FF (GE Healthcare, Fairfield, Conn.) column which was equilibrated with equilibration buffer (2.5M ammonium acetate, 50 mM Hepes, 5 mM CaCl₂, 350 mM NaCl, 0.01% Tween 80, pH 6.9). The product was eluted with elution buffer (50 mM Hepes, 5 mM CaCl₂, 0.01% Tween 80, pH 7.4), and the eluate was concentrated by centrifugal filtration using Vivaspin (Sartorius, Goettingen, Germany) devices with 30,000 MWCO.

### Example 9

### Coupling of Aminooxy-PSA to Thrombin

To 10 mg thrombin, dissolved in 10 ml Hepes buffer pH 6 (50 mM Hepes, 5 mM CaCl₂, 150 mM NaCl, 0.01% Tween) 50 μl 10 mM sodium periodate was added. The mixture was shaken in the dark for 30 min at 4° C. and quenched for 30 min at 4° C. by the addition of 100 μl of an aqueous1M glycerol solution. Then 20.0 mg aminooxy-PSA (18.8 kD) was added and the mixture was shaken over night at 4° C. The ionic strength was increased by adding a buffer containing 8M ammonium acetate (8M ammonium acetate, 50 mM Hepes, 5 mM CaCl₂, 350 mM NaCl, 0.01% Tween 80, pH 6.9) to get a final concentration of 2.5M ammonium acetate. Next, the reaction mixture was loaded on a HiTrap Butyl FF (GE Healthcare, Fairfield, Conn.) column which was equilibrated with equilibration buffer (2.5M ammonium acetate, 50 mM Hepes, 5 mM CaCl₂, 350 mM NaCl, 0.01% Tween 80, pH 6.9). The product was eluted with elution buffer (50 mM Hepes, 5 mM CaCl₂, 0.01% Tween 80, pH 7.4), and the eluate was concentrated by centrifugal filtration using Vivaspin (Sartorius, Goettingen, Germany) devices with 30,000 MWCO.

### Example 10

### Coupling of Aminooxy-PSA to FGF

To 10 mg fibroblast growth factor (FGF), dissolved in 10 ml Hepes buffer pH 6 (50 mM Hepes, 5 mM CaCl₂, 150 mM NaCl, 0.01% Tween) 50 μl 10 mM sodium periodate was added. The mixture was shaken in the dark for 30 min at 4° C. and quenched for 30 min at 4° C. by the addition of 100 μl of an aqueous1M glycerol solution. Then 20.0 mg aminooxy-PSA (18.8 kD) was added and the mixture was shaken over night at 4° C. The ionic strength was increased by adding a buffer containing 8M ammonium acetate (8M ammonium acetate, 50 mM Hepes, 5 mM CaCl₂, 350 mM NaCl, 0.01% Tween 80, pH 6.9) to get a final concentration of 2.5M ammonium acetate. Next, the reaction mixture was loaded on a HiTrap Butyl FF (GE Healthcare, Fairfield, Conn.) column which was equilibrated with equilibration buffer (2.5M ammonium acetate, 50 mM Hepes, 5 mM CaCl₂, 350 mM NaCl, 0.01% Tween 80, pH 6.9). The product was eluted with elution buffer (50 mM Hepes, 5 mM CaCl₂, 0.01% Tween 80, pH 7.4), and the eluate was concentrated by centrifugal filtration using Vivaspin (Sartorius, Goettingen, Germany) devices with 30,000 MWCO.

### Example 11

### Coupling of Aminooxy-PSA to TGF-β

To 10 mg transforming growth factor β (TGF-β), dissolved in 10 ml Hepes buffer pH 6 (50 mM Hepes, 5 mM CaCl₂, 150 mM NaCl, 0.01% Tween) 50 μl 10 mM sodium periodate was added. The mixture was shaken in the dark for 30 min at 4° C. and quenched for 30 min at 4° C. by the addition of 100 μl of an aqueous1M glycerol solution. Then 20.0 mg aminooxy-PSA (18.8 kD) was added and the mixture was shaken over night at 4° C. The ionic strength was increased by adding a buffer containing 8M ammonium acetate (8M ammonium acetate, 50 mM Hepes, 5 mM CaCl₂, 350 mM NaCl, 0.01% Tween 80, pH 6.9) to get a final concentration of 2.5M ammonium acetate. Next, the reaction mixture was loaded on a HiTrap Butyl FF (GE Healthcare, Fairfield, Conn.) column which was equilibrated with equilibration buffer (2.5M ammonium acetate, 50 mM Hepes, 5 mM CaCl₂, 350 mM NaCl, 0.01% Tween 80, pH 6.9). The product was eluted with elution buffer (50 mM Hepes, 5 mM CaC12, 0.01% Tween 80, pH 7.4), and the eluate was concentrated by centrifugal filtration using Vivaspin (Sartorius, Goettingen, Germany) devices with 30,000 MWCO.

### Example 12

### Coupling of Aminooxy-PSA to PDGF

To 10 mg platelet derived growth factor (PDGF), dissolved in 10 ml Hepes buffer pH 6 (50 mM Hepes, 5 mM CaCl₂, 150 mM NaCl, 0.01% Tween) 50 μl 10 mM sodium periodate was added. The mixture was shaken in the dark for 30 min at 4° C. and quenched for 30 min at 4° C. by the addition of 100 μl of an aqueous1M glycerol solution. Then 20.0 mg aminooxy-PSA (18.8 kD) was added and the mixture was shaken over night at 4° C. The ionic strength was increased by adding a buffer containing 8M ammonium acetate (8M ammonium acetate, 50 mM Hepes, 5 mM CaCl₂, 350 mM NaCl, 0.01% Tween 80, pH 6.9) to get a final concentration of 2.5M ammonium acetate. Next, the reaction mixture was loaded on a HiTrap Butyl FF (GE Healthcare, Fairfield, Conn.) column which was equilibrated with equilibration buffer (2.5M ammonium acetate, 50 mM Hepes, 5 mM CaCl₂, 350 mM NaCl, 0.01% Tween 80, pH 6.9). The product was eluted with elution buffer (50 mM Hepes, 5 mM CaCl₂, 0.01% Tween 80, pH 7.4), and the eluate was concentrated by centrifugal filtration using Vivaspin (Sartorius, Goettingen, Germany) devices with 30,000 MWCO.

### Example 13

### Coupling of Aminooxy-PSA to VEGF

To 10 mg vascular endothelial growth factor (VEGF), dissolved in 10 ml Hepes buffer pH 6 (50 mM Hepes, 5 mM CaCl₂, 150 mM NaCl, 0.01% Tween) 50 μl 10 mM sodium periodate was added. The mixture was shaken in the dark for 30 min at 4° C. and quenched for 30 min at 4° C. by the addition of 100 μl of an aqueous1M glycerol solution. Then 20.0 mg aminooxy-PSA (18.8 kD) was added and the mixture was shaken over night at 4° C. The ionic strength was increased by adding a buffer containing 8M ammonium acetate (8M ammonium acetate, 50 mM Hepes, 5 mM CaCl₂, 350 mM NaCl, 0.01% Tween 80, pH 6.9) to get a final concentration of 2.5M ammonium acetate. Next, the reaction mixture was loaded on a HiTrap Butyl FF (GE Healthcare, Fairfield, Conn.) column which was equilibrated with equilibration buffer (2.5M ammonium acetate, 50 mM Hepes, 5 mM CaCl₂, 350 mM NaCl, 0.01% Tween 80, pH 6.9). The product was eluted with elution buffer (50 mM Hepes, 5 mM CaCl₂, 0.01% Tween 80, pH 7.4), and the eluate was concentrated by centrifugal filtration using Vivaspin (Sartorius, Goettingen, Germany) devices with 30,000 MWCO.

### Example 14

### Coupling of Diaminooxy (3 oxa-pentane-1,5-dioxyamine linker)-PSA to Thrombomodulin

For oxidation of Thrombomodulin, NaIO₄ is used at a concentration of 2 mM. 3 mg of thrombomodulin was oxidized at acidic pH of 5.75 at 4° C. for 30 minutes then oxidation is stopped by adding NaHSO₃ to a final concentration of 2 mM. The conjugation reaction is carried out using the oxidized Thrombomodulin with diaminooxy PSA polymer (23 kDa). The final concentration of polymer in the reaction mixture is determined. The final pH of reaction mixture was around 5.75. Sodium cyanoborohydride was added to the reaction mixture to a concentration of 50 mM or 3.17 mg/ml. The reaction was carried out at 4° C. for 2 hours. Conjugates were characterized using SDS PAGE and western blotting. A shift in the band for the conjugate in SDS PAGE is indicative of a positive result.

### Example 15

### Determination of Thrombopoietin (TPO) Binding Affinity by Surface Plasmon Resonance (SPR)

TPO binding affinity is analyzed using a Biacore instrument (GE Healthcare, Uppsala, Sweden) as follows:

TPO is immobilized at three densities on the flow cells of a CMS biosensor chip. Investigational TPO samples are diluted to a series of seven dilutions (0.1 to 50 nM X according to the given protein values) with running buffer (10 mM Hepes, 150 mM NaCl, 0.05% Surfactant P20, pH 7.4), then applied to the chip using “single cycle” mode with a constant flow rate of 50 μL/min. Time for association is 4 min and that for dissociation was 10 min. After each cycle, TPO is removed from the chip (“regeneration”) and the experiment repeated with a new TPO sample. Association and dissociation constants are determined using the Langmuir model of the ‘Bioevaluation’ program. The following kinetic parameters are determined: Association rate constant ka, dissociation rate constant kd and equilibrium dissociation constant KD. Binding is also determined by evaluating Rmax, the calculated maximum binding at saturation. The kinetic results are calculated from the mean of the three different TPO immobilization levels.

Biacore technology is used to determine the kinetics of the complex formation between TPO and Tumor Necrosis Factor Receptor. For this purpose, TPO is immobilized onto three different levels on the sensor chip surface and the investigational TPO and thrombopoietin receptor rebuffered into the buffer and samples injected at five different concentrations in a single cycle mode. Association and dissociation constants are determined, assuming a homogeneous 1:1 interaction between the immobilized TPO and thrombopoietin receptor, using the Langmuir model of the “Bioevaluation” program of the Biacore T200 equipment.

The kinetic parameters describing the TPO-thrombopoietin receptor interaction such as the association rate constant (ka), the dissociation rate constant (kd), and the equilibrium dissociation constant (=kd/ka), etc. are determined and subjected to further evaluation and data comparison. Data from the PSA-modified protein is compared to unmodified.

### Example 16

### Determination of CD20 Binding Affinity by Surface Plasmon Resonance (SPR)

CD20 binding affinity is analyzed using a Biacore instrument (GE Healthcare, Uppsala, Sweden) as follows:

CD20 is immobilized at three densities on the flow cells of a CMS biosensor chip. Investigational TPO samples are diluted to a series of seven dilutions (0.1 to 50 nM X according to the given protein values) with running buffer (10 mM Hepes, 150 mM NaCl, 0.05% Surfactant P20, pH 7.4), then applied to the chip using “single cycle” mode with a constant flow rate of 50 μL/min. Time for association is 4 min and that for dissociation was 10 min. After each cycle, TPO is removed from the chip (“regeneration”) and the experiment repeated with a new TPO sample. Association and dissociation constants are determined using the Langmuir model of the ‘Bioevaluation’ program. The following kinetic parameters are determined: Association rate constant ka, dissociation rate constant kd and equilibrium dissociation constant KD. Binding is also determined by evaluating Rmax, the calculated maximum binding at saturation. The kinetic results are calculated from the mean of the three different TPO immobilization levels.

Biacore technology is used to determine the kinetics of the complex formation between CD20 and the CD20 Receptor. For this purpose, CD20 is immobilized onto three different levels on the sensor chip surface and the investigational CD20 and CD20 receptor rebuffered into the buffer and samples injected at five different concentrations in a single cycle mode. Association and dissociation constants are determined, assuming a homogeneous 1:1 interaction between the immobilized CD20 and CD20 receptor, using the Langmuir model of the “Bioevaluation” program of the Biacore T200 equipment.

The kinetic parameters describing the CD20-CD20 receptor interaction such as the association rate constant (ka), the dissociation rate constant (kd), and the equilibrium dissociation constant (=kd/ka), etc. are determined and subjected to further evaluation and data comparison. Data from the PSA-modified protein is compared to unmodified.

### Example 17

### Determination of Thrombin Binding Affinity by Surface Plasmon Resonance (SPR)

Thrombin binding affinity is analyzed using a Biacore instrument (GE Healthcare, Uppsala, Sweden) as follows:

Thrombin is immobilized at three densities on the flow cells of a CMS biosensor chip. Investigational Thrombin samples are diluted to a series of seven dilutions (0.1 to 50 nM X according to the given protein values) with running buffer (10 mM Hepes, 150 mM NaCl, 0.05% Surfactant P20, pH 7.4), then applied to the chip using “single cycle” mode with a constant flow rate of 50pt/min. Time for association is 4 min and that for dissociation was 10 min. After each cycle, Thrombin is removed from the chip (“regeneration”) and the experiment repeated with a new Thrombin sample. Association and dissociation constants are determined using the Langmuir model of the ‘Bioevaluation’ program. The following kinetic parameters are determined: Association rate constant ka, dissociation rate constant kd and equilibrium dissociation constant KD. Binding is also determined by evaluating Rmax, the calculated maximum binding at saturation. The kinetic results are calculated from the mean of the three different TNF immobilization levels.

Biacore technology is used to determine the kinetics of the complex formation between Thrombin and Thrombomodulin. For this purpose, Thrombin is immobilized onto three different levels on the sensor chip surface and the investigational Thrombin and Thrombomodulin (or an analog, e.g. Solulin) buffered into the buffer and samples injected at five different concentrations in a single cycle mode. Association and dissociation constants are determined, assuming a homogeneous 1:1 interaction between the immobilized Thrombin and Thrombomodulin, using the Langmuir model of the “Bioevaluation” program of the Biacore T200 equipment.

The kinetic parameters describing the TNF-Tumor Necrosis Factor Receptor interaction such as the association rate constant (ka), the dissociation rate constant (kd), and the equilibrium dissociation constant (=kd/ka), etc. are determined and subjected to further evaluation and data comparison. Data from the PSA-modified protein is compared to unmodified.

### Example 18

### Determination of Tumor Necrosis Factor (TNF) Binding Affinity by Surface Plasmon Resonance (SPR)

TNF binding affinity is analyzed using a Biacore instrument (GE Healthcare, Uppsala, Sweden) as follows:

TNF is immobilized at three densities on the flow cells of a CMS biosensor chip. Investigational TNF samples are diluted to a series of seven dilutions (0.1 to 50 nM X according to the given protein values) with running buffer (10 mM Hepes, 150 mM NaCl, 0.05% Surfactant P20, pH 7.4), then applied to the chip using “single cycle” mode with a constant flow rate of 50 μL/min. Time for association is 4 min and that for dissociation was 10 min. After each cycle, TNF is removed from the chip (“regeneration”) and the experiment repeated with a new TNF sample. Association and dissociation constants are determined using the Langmuir model of the ‘Bioevaluation’ program. The following kinetic parameters are determined: Association rate constant ka, dissociation rate constant kd and equilibrium dissociation constant KD. Binding is also determined by evaluating Rmax, the calculated maximum binding at saturation. The kinetic results are calculated from the mean of the three different TNF immobilization levels.

Biacore technology is used to determine the kinetics of the complex formation between TNF and Tumor Necrosis Factor Receptor. For this purpose, TNF is immobilized onto three different levels on the sensor chip surface and the investigational TNF and Tumor Necrosis Factor Receptor rebuffered into the buffer and samples injected at five different concentrations in a single cycle mode. Association and dissociation constants are determined, assuming a homogeneous 1:1 interaction between the immobilized TNF and Tumor Necrosis Factor Receptor, using the Langmuir model of the “Bioevaluation” program of the Biacore T200 equipment.

The kinetic parameters describing the TNF—Tumor Necrosis Factor Receptor interaction such as the association rate constant (ka), the dissociation rate constant (kd), and the equilibrium dissociation constant (=kd/ka), etc. are determined and subjected to further evaluation and data comparison. Data from the PSA-modified protein is compared to unmodified.

### Example 19

### Determination of Thrombin Activity Upon Binding Thrombomodulin by Activated Protein C Assay

The following materials are utilized, with sources: C57BL6J 6-8 week old male mice are obtained, e.g from Jackson Laboratories, human APC can be obtained from Haemotologic Technologies (Essex Junction, Vt., USA), Spectrazyme PCa from American Diagnostica, bovine thrombin from GE Amersham Biosciences, recombinant Hirudin from EMD Chemicals, benzamidine HCl hydrate and bovine serum albumin type V from Fisher Scientific, heparin from porcine intestinal mucosa from Sigma, 96-well Costar (cat# 3595) plates for in vitro experiments and 8-well EIA/RIA Corning strips (cat# 2590) for in vivo experiments from Fisher Scientific, and mouse monoclonal antibodies against human APC can be obtained from commercial sources. Many current assays which use a chromogenic determination of the thrombomodulin activity are based on Salem et al., Journal of Biological Chemistry, Vol. 259, No. 19, pp. 12246-12251 (1984) incorporated by reference herein. Also, the commercially available chromogenic assay BIOPHEN Protein C 2.5 is also used is some experiments and can be obtained from Hyphen BioMed (West Chester, Ohio).

Human protein C dose response in vitro. Human protein C (32.25, 64.5 and 322.5 nM) is incubated with sTM (29.3 nM) and bovine thrombin (7.36 nM) in 0.1 mL PBS (w/Ca²⁺), for 10 min in a 1.7 mL microcentrifuge tube. A 25 μl aliquot is removed and added to 25 μl recombinant hirudin [0.25U/μl] to quench thrombin activity in each well within a 96-well plate. A 50 μl solution of Spectrazyme (2 μM) is added and the OD at 60 min. is determined. Values for the 60 min time point from three independent experiments are averaged, N=3±SD. The molarities of human protein C and bovine thrombin are calculated based on MWs of 62 and 36.7 kDa, respectively.

Thrombomodulin dose response in vitro. Human protein C (64.5 nM) is incubated with sTM (0.293, 2.93, 29.3 and 1470 nM) and bovine thrombin (7.36 nM) in 0.1 mL PBS (w/Ca²⁺) for 10 min in a 1.7 mL microcentrifuge tube. A 25 μl aliquot is removed and placed in each well within a 96-well plate, 25 μl recombinant hirudin [0.25U/μl] and a 50 μl solution of Spectrazyme (2 μM) are added, and the OD at 60 min. is determined. Values for independent experiments at the 60 min time point were averaged, N=3±SD.

Human protein C dose response in vivo. Assuming an average blood volume of 1.5 mL for a 25 g mouse. sTM (29.3 nM) and bovine thrombin (7.36 nM) were pre-incubated for 10 min and injected into the jugular vein of C57BL6J mice. Immediately thereafter, human protein C (32.25, 64.5 or 322.5 nM) is injected into the contralateral jugular vein. Ten min later, blood is collected from the inferior vena cava in 3.8% sodium citrate and benzamidine HCl (v/v 2:1), with 1000 of the sodium citrate mix added per 6000 of collected blood. Blood is spun at 1500xg for 5 min. Plasma was collected and flash frozen in liquid nitrogen and stored at −80° C. until use.

Thrombomodulin dose response in vivo. sTM (0.293, 2.93, 29.3 and 1470 nM) and bovine thrombin (7.36 nM) are pre-incubated for 10 min before injection into the jugular vein of C57BL6J mice. Immediately thereafter, human protein C (64.5 nM) is injected into the contralateral jugular vein. Ten min later, blood was collected from the inferior vena cava in 3.8% sodium citrate and benzamidine HC1 (v/v 2:1). 100 μl of the sodium citrate:benzamidine HCl mix are added per 600 μl of blood. Plasma was prepared as above. Suitable and related PKC assay methods are also described in Camemolla, R., et al., J Immunol Methods., 2012 Oct. 31; 384, (1-2), 21-24, and Xu, H., et al, C. J Biol Chem 2005;280(9):7956-7961, incorporated by reference herein.

### Example 20

### Human APC Detection by Antibody-Based ELISA

Anti-human APC mAb 1555 (5 μg/mL) in 20 mM Hepes, 150NaCl, 5 mM CaCl₂, pH7.5 are allowed to bind overnight at 4° C. to high-binding EIA/RIA 8-well strips. Wells are washed twice with 20 mM Hepes, 150NaCl, 5 mM CaCl₂, 0.05% Tween-20, pH7.5 and blocked with 20 mM Hepes, 150NaCl, 5 mM CaCl₂, 1% BSA, pH7.5 for 1 hr at room temperature. Wells are then washed 2 times for 5 min each. Human APC standards [0-3200 ng/mL] were prepared in mouse plasma diluted 1:4 in 20 mM Hepes, 150NaCl, 15 mM CaCl₂, 0.1% BSA, 2U/mL heparin, 20 mM benzamidine HCl, pH7.5 (100 μl/well) Human APC standards spiked with human APC antibody [50 μg/mL] to lower background (antibody non-specificity) are prepared in parallel. Plasma samples from mice given various doses of human PC or sTM are prepared identically to standards. Standards and samples are incubated at room temperature for 1 hr, washed 4 times for 5 min each. A 100 μl solution of 1 μM Spectrazyme (diluted 1:10 in coating buffer) is added to each well and the OD at 405 nm was measured every min for 2 hrs. Raw values for human APC is substracted from human APC values obtained in the presence of excess anti-human APC mAb [50 μg/mL]. APC values are then extrapolated from the linear equation based on the standard curve. Human APC values [ng/mL] are reported as the average of N=3-4±SD.

### Example 21

### PSA-Thrombomodulin Analog (Solulin) Activity in a Thrombotic Stroke Model

In this experiment, various PSA-thrombomodulin made according to the invention are tested and compared for biological activity in a mouse thrombotic stroke model. The experiments are performed according to methods described in Su, E. J., et al., 2011, Journal of Thrombosis and Haemostasis, 9, 1174-1182, DOI: 10.1111/j.1538-7836.2011.04269.x, incorporated by reference herein. The following materials are utilized, with sources: C57BL6J 6-8 week old male mice are obtained, e.g from Jackson Laboratories, human APC can be obtained from Haemotologic Technologies (Essex Junction, Vt., USA), Spectrazyme PCa.

Ischemic stroke model. Male (10 weeks) are anesthetized with chloral hydrate (450 mg kg-1) and placed securely under a dissecting microscope. The left MCA is exposed as described, and a laser Doppler flow probe (Type N: Transonic Systems, Ithaca, N.Y., USA) is placed on the surface of the cerebral cortex located 1.5 mm dorsal median from the bifurcation of MCA. The probe is connected to a flowmeter (Transonic model BLF21) and relative tissue perfusion units (TPU) was recorded with a continuous data acquisition program (windaq, dataq, Akron, Ohio, USA). Rose Bengal (RB) (Fisher) is diluted to 10 mg mL-1 in Lactate Ringer's and then injected into the tail vein (50 mg kg-1). A 3.5-mW green light laser (540 nm; Melles Griot, Albuquerque, NM, USA) is directed at the MCA from a distance of 6 cm, and the TPU of the cerebral cortex was recorded. Stable occlusion is achieved when the TPU drops to <20% of pre-occlusion levels and does not rebound within 10 min after Laser withdrawal.

PSA-Solulin delivery and cerebral blood flow tracing. PSA-Solulin is administered via a 26-G Abbocath®-T vascular catheter (Hospira, Lake Forest, Ill., USA) inserted into the tail vein and connected to a Genie Plus syringe pump (Kent Scientific, Torrington, Conn., USA). Mice receive either Lactate Ringer's (controls) or Solulin either 30 min before RB injection, or 30 or 60 min after MCAO. Amounts to be determined.

All cerebral blood flow (CBF) tracings will be started 10 min before RB injection and the average CBF over this time was considered 100% and used to normalize CBF. Time zero is set at RB injection. Seventy-two hours after MCAO, animals are re-anesthetized with chloral hydrate (450 mg kg-1) and the surgical site was re-exposed and the Doppler flow probe was re-attached to the same location as before to obtain 72-h CBF data.

To determine stroke volume, brains are removed and cut into 2-mm-thick coronal sections and stained with 4% 2,3,5-triphenyltetrazolium chloride (TTC) in phosphate-buffered saline (PBS) for 20 min at 37 ° C., and then fixed in 4% paraformaldehyde solution for 10 m. The sections are analyzed with nih image j using the following formula: V% stroke=Σ(Areas of lesion)/Σ(Areas of ipsilateral hemisphere)×100, where V% stroke is stroke volume calculated as percent of the ipsilateral hemisphere.

Hemoglobin assay. Twenty-four hours after MCAO, brains are removed, separated into hemispheres ipsilateral and contralateral to the MCAO, and each hemisphere was homogenized in PBS on ice. After homogenizing and mixing, samples are incubated at 23 ° C. for 5 min and then centrifuged at 25000 g at 4 ° C. for 30 m and the absorbance the supernatant is read at 410 nm and the hemoglobin is quantified relative to a purified hemoglobin standard (Sigma-Aldrich, St. Louis, Mo., USA).

Immunohistochemistry. Paraffin-embedded sections (5 ᵢim) from vehicle- and Solulin-treated animals euthanized 72 h after MCAO are to be examined using the Apoptag kit (Oncor, Gaithersburg, Md., USA) according to the manufacturer's instructions.

Aspects of the present specification may also be described as follows:

A method of treating an ailment, cancer or an immune disorder, the method comprising administering an effective amount of a PSA-protein conjugate to a patient in need thereof, wherein the PSA-conjugate comprises PSA covalently linked to a protein selected from the following: Factor IX (FIX), Factor VIII (FVIII), Factor Vila (FVIIa), Von Willebrand Factor (VWF), Factor FV (FV), Factor X (FX), Factor XI (FXI), Factor XII (FXII), thrombin (FII), protein C, protein S, tPA, PAI-1, tissue factor (TF), ADAMTS 13 protease, IL-1 alpha, IL-1 β, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-β, IFN-gamma, IFN-omega, IL-7, IL-8, IL-9, IL-10, IL-12, IL-13, IL-14, IL-15, IL-16, IL-17, IL-18, IL-19, IL-20, IL-21, IL-22, IL-23, IL-24, IL-31, IL-32 alpha, IL-33, thrombopoietin (TPO), Ang-1, Ang-2, Ang-4, Ang-Y, angiopoietin-like polypeptide 1 (ANGPTL 1), angiopoietin-like polypeptide 2 (ANGPTL2), angiopoietin-like polypeptide 3 (ANGPTL3), angiopoietin-like polypeptide 4 (ANGPTL4), angiopoietin-like polypeptide 5 (ANGPTL5), angiopoietin-like polypeptide 6 (ANGPTL6), angiopoietin-like polypeptide 7 (ANGPTL7), vitronectin, vascular endothelial growth factor (VEGF), angiogenin, activin A, activin B, activin C, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, bone morphogenic protein receptor II, brain derived neurotrophic factor, cardiotrophin-1, ciliary neutrophic factor, ciliary neutrophic factor receptor, cripto, cryptic, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epidermal growth factor, epigen, epiregulin, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor 11, fibroblast growth factor 12, fibroblast growth factor 13, fibroblast growth factor 16, fibroblast growth factor 17, fibroblast growth factor 19, fibroblast growth factor 20, fibroblast growth factor 21, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein-γ, heparin binding epidermal growth factor, hepatocyte growth factor, hepatocyte growth factor receptor, hepatoma-derived growth factor, insulin-like growth factor I, insulin-like growth factor receptor, insulin-like growth factor II, insulin-like growth factor binding protein, keratinocyte growth factor, leukemia inhibitory factor, leukemia inhibitory factor receptor a, nerve growth factor nerve growth factor receptor, neuropoietin, neurotrophin-3, neurotrophin-4, oncostatin M (OSM), placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor (SCF), stem cell factor receptor, TNF, TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, thymic stromal lymphopoietin (TSLP), tumor necrosis factor receptor type I, tumor necrosis factor receptor type II, urokinase-type plasminogen activator receptor, phospholipase-activating protein (PUP), insulin, lectin ricin, prolactin, chorionic gonadotropin, follicle-stimulating hormone, thyroid-stimulating hormone, tissue plasminogen activator, IgG, IgE, IgM, IgA, and IgD, a-galactosidase, β-galactosidase, DNAse, fetuin, leutinizing hormone, estrogen, insulin, albumin, lipoproteins, fetoprotein, transferrin, thrombopoietin, urokinase, integrin, thrombin, leptin, adalimumab, denosumab, or etanercept.

A method of treating an ailment, cancer or an immune disorder, the method comprising administering an effective amount of a PSA-protein conjugate to a patient in need thereof, wherein the PSA-conjugate comprises PSA covalently linked to a protein selected from the following: factor IX (FIX), Factor VIII (FVIII), Factor VIIa (FVIIa), von Willebrand Factor (VWF), Factor FV (FV), Factor X (FX), Factor XI (FXI), Factor XII (FXII), thrombin (FII), protein C, protein S, tPA, PAI-1, tissue factor (TF), ADAMTS 13 protease, IL-1 alpha, IL-1 β, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-β, IFN-gamma, IFN-omega, IL-7, IL-8, IL-9, IL-10, IL-12, IL-13, IL-14, IL-15, IL-16, IL-17, IL-18, IL-19, IL-20, IL-21, IL-22, IL-23, IL-24, IL-31, IL-32 alpha, IL-33, thrombopoietin (TPO), Ang-1, Ang-2, Ang-4, Ang-Y, angiopoietin-like polypeptide 1 (ANGPTL1), angiopoietin-like polypeptide 2 (ANGPTL2), angiopoietin-like polypeptide 3 (ANGPTL3), angiopoietin-like polypeptide 4 (ANGPTL4), angiopoietin-like polypeptide 5 (ANGPTL5), angiopoietin-like polypeptide 6 (ANGPTL6), angiopoietin-like polypeptide 7 (ANGPTL7), vitronectin, vascular endothelial growth factor (VEGF), angiogenin, activin A, activin B, activin C, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, bone morphogenic protein receptor II, brain derived neurotrophic factor, cardiotrophin-1, ciliary neutrophic factor, ciliary neutrophic factor receptor, cripto, cryptic, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epidermal growth factor, epigen, epiregulin, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor 11, fibroblast growth factor 12, fibroblast growth factor 13, fibroblast growth factor 16, fibroblast growth factor 17, fibroblast growth factor 19, fibroblast growth factor 20, fibroblast growth factor 21, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, hepatoma-derived growth factor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neuropoietin, neurotrophin-3, neurotrophin-4, oncostatin M (OSM), placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor a, platelet derived growth factor receptor β, pre-B cell growth stimulating factor, stem cell factor (SCF), stem cell factor receptor, TNF, TNFO, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, thymic stromal lymphopoietin (TSLP), tumor necrosis factor receptor type I, tumor necrosis factor receptor type II, urokinase-type plasminogen activator receptor, phospholipase-activating protein (PUP), insulin, lectin ricin, prolactin, chorionic gonadotropin, follicle-stimulating hormone, thyroid-stimulating hormone, tissue plasminogen activator, IgG, IgE, IgM, IgA, and IgD, α-galactosidase, β-galactosidase, DNAse, fetuin, leutinizing hormone, estrogen, insulin, albumin, lipoproteins, fetoprotein, transferrin, thrombopoietin, urokinase, integrin, thrombin, leptin, Humira (adalimumab), Prolia (denosumab), or a biologically active fragment, derivative or variant thereof

A method of treating HIV, the method comprising administering an effective amount of a PSA-protein conjugate to a patient in need thereof, wherein the PSA-conjugate comprises PSA covalently linked to a protein that binds to an HIV envelope glycoprotein at the gp120-gp41 interface.

A method of treating an ailment, cancer or an immune disorder, the method comprising administering an effective amount of a PSA-protein conjugate to a patient in need thereof, wherein the PSA-conjugate comprises PSA covalently linked to a protein selected from the following: carbonic anhydrase IX, alpha-fetoprotein. alpha-actinin-4, A3 (antigen specific for A33 antibody), ART-4, B7, Ba-733, BAGE, BrE3-antigen, CA125, CAMEL, CAP-1, CASP-8/m, CCCL19, CCCL21, CD1, CD1a, CD2, CD3, CD4, CDS, CD8, CD1-1A, CD14, CD15, CD16, CD18, CD19, CD20, CD2I, CD22, CD23, CD25, CD29, CD30,CD32b, CD33, CD37, CD38, CD40, CD40L, CD45, CD46, CD54, CD55, CD59, CD64, CD66a-e, CD67, CD70, CD74, CD79a, CD80, CD83, CD95, CD126, CD133, CD138, CD147, CD154, CDC27, CDK-4/m, CDK 2A, CXCR4, CXCR7, CXCL12, HIF-1-alpha, colon-specific antigen-p (CSAp), CEA (CEACAM5), CEACAM6, c-met, DAM, EGFR, EGERvIII, EGP-1, EGP-2, ELF2-M, Ep-CAM,Flt-1, Flt-3, folate receptor, G250 antigen, GAGE, GROB, HLA-DR, HM1.24, human chorionic gonadotropin (HCG) and its subunits, HER2/neu, HMGB-1, hypoxia inducible factor (HIF-1), HSP70-2M, HST-2or 1a, IGF-1R, IFN-gamma, IFN-alpha, IFN-β, IL-2, IL-4R, IL-6R, IL-13R, IL-15R, IL-17R, IL-18R, IL-6, IL-8, IL-12, IL-15, IL-17, IL-18, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, ‘TAG-3, mCRP, MCP-1, MIP-1A, MIP-1B, MIF, MUC1, MUC2, MUC3, MUC4, MUC5, MUM-1/2, MUM-3, NCA66, NCA95, NCA90, pancreatic cancer mucin, placental growth factor, p53, PLAGL2, prostatic acid phosphatase, PSA, PRAME, PSMA, P1GF, ILGF, ILGF-1R, IL-6, 1L-25, RS5, RANTES, T101, SAGE, S100, survivin, survivin-2B, TAC, TAG-72, tenascin, TRAIL receptors, TNF-alpha, Tn-antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bc1-6, Kras, cMET, an oncogene marker and an oncogene product.

A method of treating an ailment, cancer or an immune disorder, the method comprising administering an effective amount of a PSA-protein conjugate to a patient in need thereof, wherein the PSA-conjugate comprises PSA covalently linked to an antibody or binding protein that recognizes me sothelin.

A method of treating an ailment, cancer or an immune disorder, the method comprising administering an effective amount of a PSA-protein conjugate to a patient in need thereof, wherein the PSA-conjugate comprises PSA covalently linked to a protein selected from the following: an enzyme from the esterase group comprising a metalloproteinase, a subtilase, or a lipase, triacylglycerol lipase, subtilase, metalloproteinase, cholinesterase, acetylcholinesterase, butyrylcholinesterase, trypsin, subtilisin, thermolysin, or CT, cholinesterase, acetylcholinesterase, butyrylcholinesterase, subtilase, subtilisin, therinolysin, lipase, triacylglycerol lipase, metalloproteinase, chymotrypsin, -chymotrypsin, or trypsin, an enzyme polymer conjugate such as an esterase-polymer conjugate comprising a chymotrypsin-pDMAEMA (CT-pDMAEMA) conjugate, a metalloproteinase-pOEGMA conjugate, a thermolysin-pOEGMA conjugate, a subtilisin-ionic liquid polymer conjugate, a subtilase-ionic liquid polymer conjugate, or a lipase-pDMAA conjugate.

A method of treating an ailment, cancer or an immune disorder, the method comprising administering an effective amount of a PSA-protein conjugate to a patient in need thereof, wherein the PSA-conjugate comprises PSA covalently linked to an antibody-drug conjugate comprising the following antibodies: anti-HER2 monoclonal antibody such as trastuzumab and pertuzumab, anti-CD20 monoclonal antibody such as rituximab, ofatumumab, tositumomab and ibritumomab, anti-CA125 monoclonal antibody such as oregovomab, anti-EpCAM (17-1A) monoclonal antibody such as edrecolomab, anti-EGFR monoclonal antibody such as cetuximab, panitumumab and nimotuzumab, anti-CD30 monoclonal antibody such brentuximab, anti-CD33 monoclonal antibody such as gemtuzumab and huMy9-6, anti-vascular integrin alpha-v β-3 monoclonal antibody such as etaracizumab, anti-CD52 monoclonal antibody such as 5 alemtuzumab, anti-CD22 monoclonal antibody such as epratuzumab, anti-CEA monoclonal antibody such as labetuzumab, anti-CD44v6 monoclonal antibody such as bivatuzumab, anti-FAP monoclonal antibody such as sibrotuzumab, anti-CD19 monoclonal antibody such as huB4, anti-CanAg monoclonal antibody such as huC242, anti-CD56 monoclonal antibody such huN901, anti-CD38 monoclonal antibody such as daratumumab, anti-CA6 monoclonal antibody 10 such as DS6, anti-IGF-IR monoclonal antibody such as cixutumumab and 3B7, anti-integrin monoclonal antibody such as CNTO 95, and anti-syndecan-1 monoclonal antibody such as B-B4.

A method of treating an ailment, cancer or an immune disorder, the method comprising administering an effective amount of a PSA-protein conjugate to a patient in need thereof, wherein the PSA-conjugate comprises PSA covalently linked to an binding molecule-drug conjugate comprising the following binding compounds that are not antibodies: binding proteins other than antibodies can also be used as the cell-binding ligand for the ligand-drug conjugates including but not limited to interferons such as IFN-a, IFN-f3, and IFN-y, transferrins, epidermal growth factors (EGF) and EGF-like domains, gastrin-releasing peptides (GRP), platelet-derived growth factors (PDGF), transforming growth factors (TGF), vaccinia growth factor (VGF), insulin and insulin-like growth factors (IGF) such as IGF-1 and IGF-2, other suitable hormones such as thyrotropin releasing hormones (TRH), melanocyte-stimulating 30 hormones (MSH), steroid hormones (for example, estrogen and androgen), and somatostatin, lymphokines such as IL-2, IL-3, IL-4, and IL-6, colony-stimulating factors (CSF) such as G-CSF, M-CSF and GM-CSF, bombesin, gastrin, and folic acid.

A method of treating an ailment, cancer or an immune disorder, the method comprising administering an effective amount of a PSA-thrombin conjugate to a patient in need thereof, wherein the PSA-thrombin conjugate comprises PSA covalently linked to thrombin.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

TABLE 1
Sequences of Protein Coniu2ates

| SEQ | | |
|---|---|---|
| ID | Name | |
| NO | (description) | Amino Acid Sequence |
| 1 | E124 | MADSVKTFLQDLARGIKDSI |
| | | WGICTISKLDARIQQKREEQ |
| | | RRRRASSVLAQRRAQSIERK |
| | | QESEPRIVSRIFQCCAWNGG |
| | | VFWFSLLLFYRVFIPVLQSV |
| | | TARIIGDPSLHGDVWSWLEF |
| | | FLTSIFSALWVLPLFVLSKV |
| | | VNAIWFQDIADLAFEVSGRK |
| | | PHPFPSVSKIIADMLFNLLL |
| | | QALFLIQGMFVSLFPIHLVG |
| | | QLVSLLHMSLLYSLYCFEYR |
| | | WFNKGIEMHQRLSNIERNWP |
| | | YYFGFGLPLAFLTAMQSSYI |
| | | ISGCLFSILFPLFIISANEA |
| | | KTPGKAYLFQLRLFSLVVFL |
| | | SNRLFHKTVYLQSALSSSTS |
| | | AEKFPSPHPSPAKLKATAGH |
| 2 | PRUP3 | ASSNGIRNVNNLARTPDRQA |
| | | C |
| 3 | TNF | VPAQWFPRSIPEPSNLCQPR |
| | | EYYDERAQRRCSQCPPGCRA |
| | | KSFCNETSDTVCVPCEDSTY |
| | | TQLWNWLPECLSCGSRCSTG |
| | | QVETQACTLKQNRICTCEPG |
| | | RYCILPRQEGCQVCGLLRKC |
| | | PPGFGVAKPGTATSNWCA |
| 4 | p38 gamma | YGRKKRRQRRRARVPKETAL |
| | protein | |
| | inhibitor | |
| 5 | Anti-cancer | RPMRLESFSACIWVKATDVL |
| | | NKTILFSY GTKRN PYEI |
| 6 | Anti-cancer | GGGFDETLAFSGRLTGFNIW |
| | | DSVLSNEEIRETGGAESCHI |
| 7 | Tau protein | KHQPGGG |
| 8 | Tau protein | KHVPGGG |
| 9 | Tau protein | HHKPGGG |
| 10 | Tau protein | THVPGGG |
| 11 | SERCA calcium | MAEKESTSPHLMVPILLLVG |
| | pump | WIVGCIIVIYIVFF |
| 12 | SERCA calcium | MAEKAESTSPHLMVPILLLV |
| | pump | GWIVGCIIVIYIVFF |
| 13 | SERCA calcium | MAEKESTSPHLIVPILLLVG |
| | pump | WIVGCIIVIYIVFF |
| 14 | SERCA calcium | MAEKAESTSPHLIVPILLLV |
| | pump | GWIVGCIIVIYIVFF |
| 15 | excitatory | YEKLLDTEI |
| | neurotoxicity | |
| 16 | GLP1 | HSQGTFTSDYSKYLDSRRAQ |
| | | DFVQWLMNT |
| 17 | GLP1 | EEPSSGAPPPS |
| 18 | GLP1 | EPSSGAPPPS |
| 19 | GLP1 | GAPPPS |
| 20 | GLP1 | GGPSSGAPPPS |
| 21 | GLP1 | GPSSGAPPPS |
| 22 | GLP1 | KRNKNPPPS |
| 23 | GLP1 | KRNKNPPS |
| 24 | GLP1 | KRNKPPIA |
| 25 | GLP1 | KRNKPPPA |
| 26 | GLP1 | KRNKPPPS |
| 27 | GLP1 | KSSGKPPPS |
| 28 | GLP1 | PESGAPPPS |
| 29 | GLP1 | PKSGAPPPS |
| 30 | GLP1 | PKSKAPPPS |
| 31 | GLP1 | PKSKAPPPS |
| 32 | GLP1 | PKSKEPPPS |
| 33 | GLP1 | PKSKEPPPS |
| 34 | GLP1 | PKSKQPPPS |
| 35 | GLP1 | PKSKSPPPS |
| 36 | GLP1 | PKSKSPPPS |
| 37 | GLP1 | PRNKNNPPS |
| 38 | GLP1 | PSKGAPPPS |
| 39 | GLP1 | PSSGAPPPSE |
| 40 | GLP1 | PSSGAPPPS |
| 41 | GLP1 | PSSGAPPPS |
| 42 | GLP1 | PSSGAPPPSS |
| 43 | GLP1 | PSSGEPPPS |
| 44 | GLP1 | PSSGKKPPS |
| 45 | GLP1 | PSSGKPPPS |
| 46 | GLP1 | PSSGKPPPS |
| 47 | GLP1 | PSSGSPPPS |
| 48 | GLP1 | PSSKAPPPS |
| 49 | GLP1 | PSSKEPPPS |
| 50 | GLP1 | PSSKGAPPPS |
| 51 | GLP1 | PSSKQPPPS |
| 52 | GLP1 | PSSKSPPPS |
| 53 | GLP1 | SGAPPPS |
| 54 | GLP1 | SSGAPPPS |
| 55 | TP4 | FIHHIIGGLFSAGKAIHRLI |
| | | RRRRR |
| 56 | BB barrier | LRVRLASHLRKLRKRLLRDA |
| | agent | |
| 57 | Telomerase | RRRGGDASRSLPLPKRPRR |
| | inhibitor | |
| 58 | Telomerase | RRRGGEASRSLPLPKRPRR |
| | inhibitor | |
| 59 | interferon | SSGSSSSSSSKAPPPSLPSP |
| | | SRLPGPSDTPILPQNGS |
| 60 | Integrin | VGDLTYLK |
| | binding | |
| 61 | Integrin | VGDLTYLKK |
| | binding | |
| 62 | Ziconotide | CKGKGAKCSRLMYDCCTGSC |
| | | RSGKC |

What is claimed is:

1. A method of treating an ailment, the method comprising administering an effective amount of a PSA-protein conjugate to a patient in need thereof, wherein the PSA-conjugate comprises PSA covalently linked to a protein selected from the following: factor IX (FIX), Factor VIII (FVIII), Factor VIIa (FVIIa), von Willebrand Factor (VWF), Factor FV (FV), Factor X (FX), Factor XI (FXI), Factor XII (FXII), thrombin (FII), protein C, protein S, tPA, PAI-1, tissue factor (TF), ADAMTS 13 protease, IL-1 alpha, IL-1 β, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-β,l IFN-gamma, IFN-omega, IL-7, IL-8, IL-9, IL-10, IL-12, IL-13, IL-14, IL-15, IL-16, IL-17, IL-18, IL-19, IL-20, IL-21, IL-22, IL-23, IL-24, IL-31, IL-32 alpha, IL-33, thrombopoietin (TPO), Ang-1, Ang-2, Ang-4, Ang-Y, angiopoietin-like polypeptide 1 (ANGPTL1), angiopoietin-like polypeptide 2 (ANGPTL2), angiopoietin-like polypeptide 3 (ANGPTL3), angiopoietin-like polypeptide 4 (ANGPTL4), angiopoietin-like polypeptide 5 (ANGPTL5), angiopoietin-like polypeptide 6 (ANGPTL6), angiopoietin-like polypeptide 7 (ANGPTL7), vitronectin, vascular endothelial growth factor (VEGF), angiogenin, activin A, activin B, activin C, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, bone morphogenic protein receptor II, brain derived neurotrophic factor, cardiotrophin-1, ciliary neutrophic factor, ciliary neutrophic factor receptor, cripto, cryptic, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epidermal growth factor, epigen, epiregulin, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor 11, fibroblast growth factor 12, fibroblast growth factor 13, fibroblast growth factor 16, fibroblast growth factor 17, fibroblast growth factor 19, fibroblast growth factor 20, fibroblast growth factor 21, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, hepatoma-derived growth factor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neuropoietin, neurotrophin-3, neurotrophin-4, oncostatin M (OSM), placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor (SCF), stem cell factor receptor, TNF, TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β, binding protein I, transforming growth factor β binding protein II, transforming growth factor β binding protein III, thymic stromal lymphopoietin (TSLP), tumor necrosis factor receptor type I, tumor necrosis factor receptor type II, urokinase-type plasminogen activator receptor, phospholipase-activating protein (PUP), insulin, lectin ricin, prolactin, chorionic gonadotropin, follicle-stimulating hormone, thyroid-stimulating hormone, tissue plasminogen activator, IgG, IgE, IgM, IgA, and IgD, a-galactosidase, β-galactosidase, DNAse, fetuin, leutinizing hormone, estrogen, insulin, albumin, lipoproteins, fetoprotein, transferrin, thrombopoietin, urokinase, integrin, thrombin, leptin, Humira (adalimumab), Prolia (denosumab), or a biologically active fragment, derivative or variant thereof

2. A method of treating HIV, the method comprising administering an effective amount of a PSA-protein conjugate to a patient in need thereof, wherein the PSA-conjugate comprises PSA covalently linked to a protein that binds to an HIV envelope glycoprotein at the gp120-gp41 interface.

3. A method of treating an ailment, the method comprising administering an effective amount of a PSA-protein conjugate to a patient in need thereof, wherein the PSA-conjugate comprises PSA covalently linked to a protein selected from the following: carbonic anhydrase IX, alpha-fetoprotein, alpha-actinin-4, A3 (antigen specific for A33 antibody), ART-4, B7, Ba-733, BAGS, BrE3-antigen, CA125, CAMEL, CAP-1, CASP-8/m, CCCL19, CCCL21, CD1, CD1a, CD2, CD3, CD4, CDS, CD8, CD1-1A, CD14, CD15, CD16, CD18, CD19, CD20, CD21, CD22, CD23, CD25, CD29, CD30,CD32b, CD33, CD37, CD38, CD40, CD40L, CD45, CD46, CD54, CD55, CD59, CD64, CD66a-e, CD67, CD70, CD74, CD79a, CD80, CD83, CD95, CD126, CD133, CD138, CD147, CD154, CDC27, CDK-4/m, CDK 2A, CXCR4, CXCR7, CXCL12, HIF-1-alpha, colon-specific antigen-p (CSAp), CEA (CEACAM5), CEACAM6, c-met, DAM, EGFR, EGFRvIII, EGP-I, EGP-2, ELF2-M, Ep-CAM, Flt-1, Flt-3, folate receptor, G250 antigen, GAGE, GROB, HLA-DR, HM1.24, human chorionic gonadotropin (HCG) and its subunits, HER2/neu, HMGB-1, hypoxia inducible factor (HIF-1), HSP70-2M, HST-2or la, IGF-1R, IFN-gamma, IFN-alpha, IFN-β, IL-2, IL-4R, IL-6R, IL-13R, IL-15R, IL-17R, IL-18R, IL-6, IL-8, 1L-12, IL-15, IL-17, IL-18, IL-25, insulin-like growth factor-1 (IGF-1), KC4-antigen, KS-1 -antigen, KS1-4, Le-Y, LDRIFUT, macrophage migration inhibitory factor (MIF), MAGE, MAGE-3, MART-4, MART-2, NY-ESO-1, ‘TRAG-3 mCRP, MCP-1, MIP-1A, MIP-1B, MIF, MUC1, MUC2, MUC3, MUC4, MUC5, MUM-1/2, MUM-3, NCA66, NCA95, NCA90, pancreatic cancer mucin, placental growth factor, p53, PLAGL2, prostatic acid phosphatase, PSA, PRAME, PSMA, P1GF, ILGF, ILGF-1R, L 6, IL-25, RS5, RANTES, T101, SAGE, S100, survivin, survivin-2B, TAC, TAG-72, tenascin, TRAIL receptors, TNF-alpha, Tn-antigen, Thomson-Friedenreich antigens, tumor necrosis antigens. VEGFR, ED-B fibronectin, WT-1, 17-1A-antigen, complement factors C3, C3a, C3b, C5a, C5, an angiogenesis marker, bbl-2, bcl-6, Kras, cMET, an oncogene marker and an oncogene product.

4. A method of treating an ailment, the method comprising administering an effective amount of a PSA-protein conjugate to a patient in need thereof, wherein the PSA-conjugate comprises PSA covalently linked to an antibody or binding protein that recognizes mesothelin.

5. A method of treating an ailment, the method comprising administering an effective amount of a PSA-protein conjugate to a patient in need thereof, wherein the PSA-conjugate comprises PSA covalently linked to a protein selected from the following: an enzyme from the esterase group comprising a metalloproteinase, a subtilase, or a lipase, triacylglycerol lipase, subtilase, metalloproteinase, cholinesterase, acetylcholinesterase, butyrylcholinesterase, trypsin, subtilisin, thermolysin, or CT, cholinesterase, acetylcholinesterase, butyrylcholinesterase, subtilase, subtilisin, thermolysin, lipase, triacylglycerol lipase, metalloproteinase, chymottypsin, -chymotrypsin, or trypsin, an enzyme polymer conjugate such as an esterase-polyiner conjugate comprising a chyinotrypsirt-pDMAEMA (CT-pDMAE.MA) conjugate, a metalloproteinase-pOEGMA conjugate, a thermolysin-pOEGMA conjugate, a subtilisin-ionic liquid polymer conjugate, a subtilase-ionic liquid polymer conjugate, or a lipase-pDMAA conjugate.

6. A method of treating an ailment, the method comprising administering an effective amount of a PSA-protein conjugate to a patient in need thereof, wherein the PSA-conjugate comprises PSA covalently linked to an antibody-drug conjugate comprising the following antibodies: anti-HER2 monoclonal antibody such as trastuzumab and pertuzumab, anti-CD20 monoclonal antibody such as rituximab, ofatumumab, tositumomab and ibritumomab, anti-CA125 monoclonal antibody such as oregovomab, anti-EpCAM (17-1A) monoclonal antibody such as edrecolomab, anti-EGFR monoclonal antibody such as cetuximab, panitumumab and nimotuzumab, anti-CD30 monoclonal antibody such brentuximab, anti-CD33 monoclonal antibody such as gemtuzumab and huMy9-6, anti-vascular integrin alpha-v 0-3 monoclonal antibody such as etaracizumab, anti-CD52 monoclonal antibody such as 5 alemtuzumab, anti-CD22 monoclonal antibody such as epratuzumab, anti-CEA monoclonal antibody such as labetuzumab, anti-CD44v6 monoclonal antibody such as bivatuzumab, anti-FAP monoclonal antibody such as sibrotuzumab, anti-CD19 monoclonal antibody such as huB4, anti-CanAg monoclonal antibody such as huC242, anti-CD56 monoclonal antibody such huN901, anti-CD38 monoclonal antibody such as daratumumab, anti-CA6 monoclonal antibody 10 such as DS6, anti-IGF-IR monoclonal antibody such as cixutumumab and 3B7, anti-integrin monoclonal antibody such as CNTO 95, and anti-syndecan-1 monoclonal antibody such as B-B4.

7. A method of treating an ailment, the method comprising administering an effective amount of a PSA-protein conjugate to a patient in need thereof, wherein the PSA-conjugate comprises PSA covalently linked to an binding molecule-drug conjugate comprising the following binding compounds that are not antibodies: binding proteins other than antibodies can also be used as the cell-binding ligand for the ligand-drug conjugates including but not limited to interferons such as IFN-a, IFN-f3, and IFN-y, transferrins, epidermal growth factors (EGF) and EGF-like domains, gastrin-releasing peptides (GRP), platelet-derived growth factors (PDGF), transforming growth factors (TGF), vaccinia growth factor (VGF), insulin and insulin-like growth factors (IGF) such as IGF-1 and IGF-2, other suitable hormones such as thyrotropin releasing hormones (TRH), melanocyte-stimulating 30 hormones (MSH), steroid hormones (for example, estrogen and androgen), and somatostatin, lymphokines such as IL-2, IL-3, IL-4, and IL-6, colony-stimulating factors (CSF) such as G-CSF, M-CSF and GM-CSF, bombesin, gastrin, and folic acid.

8. A method of treating an ailment, the method comprising administering an effective amount of a PSA-thrombin conjugate to a patient in need thereof, wherein the PSA-thrombin conjugate comprises PSA covalently linked to thrombin.

9. The method of any of claim 1, or 3-8, wherein the ailment is cancer or an autoimmune disorder.

10. A method of treating a gene expression disorder, the method comprising administering an effective amount of a PSA-nucleic acid conjugate to a patient in need thereof, wherein the PSA-conjugate comprises PSA covalently linked to an RNA oligonucleotide selected from the following:
double stranded RNA, single-stranded RNA or short interfering RNA (siRNA).

11. A method of treating a gene expression disorder, the method comprising administering an effective amount of a PSA-nucleic acid conjugate to a patient in need thereof, wherein the PSA-conjugate comprises PSA covalently linked to an RNA oligonucleotide via a cleavable linker moiety.

12. A method of treating a gene expression disorder, the method comprising administering an effective amount of a PSA-nucleic acid conjugate to a patient in need thereof, wherein the PSA molecule is conjugated, optionally via a linker, to at least one RNA molecule at the RNA 3′ terminal base.

13. A method of treating a gene expression disorder, the method comprising administering an effective amount of a PSA-nucleic acid conjugate to a patient in need thereof, wherein the PSA-conjugate comprises PSA covalently linked to an RNA oligonucleotide wherein the RNA oligonucleotide comprises a coding region coding for a polypeptide or its complementary sequence, wherein the polypeptide is selected from VEGF, Apolipoprotein B, Exon 51 of dystrophin, SMN2, Transthyretin, CEP290 c.2991+1655A>G Mutation, KRAS, Complement 5 (C5) protein, EphA2, CTGF, TRPV1, LDHA, TGF-β1, Cox-2, KRAS G12D, P53, Caspase-2,kntithrombin, FANCA, Coagulation Factor VIII, Coagulation Factor IX, ANK1, PIG-A, UROD, Adenosine deaminase, JAK3, RAG1/2, Artemis, IL7R-α, IL-2Rγ, T-cell surface glycoprotein CD3 delta chain, CD3.epsilon, CDKN2, NF1, NF2, LIM kinase, elastin, ALDP, CFTR, hepcidin, ABCA3, surfactant protein B, ADAMT S13, alpha.1-antitrypsin or GAA.

14. The method of any of claims 10-13, wherein the gene expression disorder is at least one of hypertension, elevated cholesterol level, cancer, neurodegenerative disorders, mental illness, cystic fibrosis, hemophilia (or other blood clotting disease), europsychiatric disorders, such as schizophrenia, bipolar disorder, major depression, Parkinson's disease, Alzheimer's disease and autism spectrum disorders, Albinism, Angelman syndrome, Ankylosing spondylitis, Apert syndrome, Charcot-Marie-Tooth disease, Congenital adrenal hyperplasia, Cystic fibrosis, Down syndrome, Achondroplasia, Alpha-1 Antitrypsin Deficiency, Antiphospholipid Syndrome, Attention Deficit Hyperactivity Disorder, Autism, Autosomal Dominant Polycystic Kidney Disease, Charcot-Marie-Tooth Disease, Cri du Chat Syndrome, Crohn's Disease, Cystic Fibrosis, Dercum Disease, Duane Syndrome, Duchenne Muscular Dystrophy, Factor V Leiden Thrombophilia, Familial Hypercholesterolemia, Familial Mediterranean Fever, Fragile X Syndrome, Gaucher Disease, Hemochromatosis, Holoprosencephaly, Huntington's Disease, Inborn Errors of Metabolism, Klinefelter Syndrome, Marfan Syndrome, Methylmalonic Acidemia, Myotonic Dystrophy, Neurofibromatosis, Noonan Syndrome, Osteogenesis Imperfecta, Parkinson's Disease, Phenylketonuria, Poland Anomaly, Porphyria, Progeria, Retinitis Pigmentosa, Severe Combined Immunodeficiency, Sickle Cell Disease, Spinal Muscular Atrophy, Tay-Sachs Disease, Thalassemia, Trimethylaminuria, Turner Syndrome, Velocardiofacial Syndrome or Wilson Disease.
